(19) United States
(12) Patent Application Publication
(10) Pub. No.: US 2022/0344013 A1
(43) Pub. Date: Oct. 27, 2022

(54) Directing Medical Diagnosis and Intervention Recommendations

(71) Applicant: Endpoint Health Inc., Palo Alto, CA (US)

(72) Inventors: Jason Morrell Springs, Palo Alto, CA (US); Jeffrey Robert Osborn, Palo Alto, CA (US); Diego Ariel Rey, Palo Alto, CA (US); Leonardo Maestri Teixeira, Palo Also, CA (US); Emily Mary Van Ark, Palo Alto, CA (US); Rachel Elizabeth Kast, Palo Alo, CA (US); Jonathan Durbin, Palo Alto, CA (US); Rodrigo Octavio Deliberato, Palo Alto, CA (US); Maynard Joseph Gellada, Palo Alto, CA (US)

(21) Appl. No.: 17/766,022

(22) PCT Filed: Oct. 2, 2020
(86) PCT No.: PCT/US2020/053987

§ 371 Date: Apr. 1, 2022

Related U.S. Application Data
(60) Provisional application No. 62/909,534, filed on Oct. 2, 2019.

(51) Int. Cl. G16H 10/60; G16H 50/20

(52) U.S. Cl. CPC G16H 10/60; G16H 50/20; G16H 50/30

(57) ABSTRACT

A method for determining at least one of a medical diagnosis recommendation and a medical intervention recommendation for a subject. At least one of electronic health record (EHR) data and biomarker data for the subject are input into a diagnostic/intervention recommendation model that comprises parameters and a function. The parameters are identified based on a training dataset that comprises a plurality of training samples. Each training sample is associated with a retrospective subject and includes at least one of EHR data and biomarker data for the retrospective subject. The function represents a relation between the at least one of EHR data and biomarker data for the subject received as inputs to the diagnostic/intervention recommendation model, and at least one of a medical diagnosis recommendation and intervention recommendation for the subject generated as an output of the model.

### CROSS-REFERENCE TO RELATED APPLICATIONS

This application claims the benefit of and priority to U.S. Nonprovisional patent application Ser. No. 16/909,534, filed Oct. 2, 2019, which is incorporated herein by reference in its entirety for all purposes.

### BACKGROUND

The ability to quickly and accurately determine medical diagnoses and interventions, as well as the availability of medical intervention options, are paramount to the prognosis and medical outcome of subjects. This is especially true for subjects afflicted with acute conditions, such as, for example, sepsis.

A large and growing quantity of patient data, both for a subject in question as well as for prior subjects that have previously been similarly diagnosed and treated, theoretically imparts medical care providers with an abundance of information to aid in the accuracy of the diagnosis and recommendation of medical interventions for the subject. However, the sheer volume of this available patient data renders organization and processing of this data difficult. In particular, human care providers are incapable of quickly and accurately processing large quantities of medical data to ascertain patterns to be used in optimizing diagnosis and intervention of subjects.

Additionally, while a large and growing quantity of patient data exists, in some cases, this data is siloed both within and across institutions, and is not shared within and between these institutions for a plethora of reasons, including data privacy and security concerns, as well as data incompatibility. For instance, data that is common between institutions may necessitate transformation prior to comparison due to differences in method and unit of storage. As a result, not only is the quantity of data available to medical care providers at each institution decreased, but the variety of the data is decreased as well. These deficiencies in data availability and compatibility between institutions impedes the ability of care providers to leverage data to optimize diagnosis and intervention of subjects.

### SUMMARY

Disclosed herein is a method for determining a medical intervention recommendation for a subject diagnosed with a condition, the method comprising the steps of: obtaining electronic health record data for the subject; obtaining biomarker data for the subject, the biomarker data obtained from a sample from the subject; inputting, using a computer processor, the electronic health record data and the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model.

Additionally disclosed herein is a method for determining a medical intervention recommendation for a subject diagnosed with a condition, the method comprising the steps of: obtaining electronic health record data for the subject; obtaining biomarker data for the subject, the biomarker data obtained from a sample from the subject; inputting, using a computer processor, the electronic health record data and the biomarker data for the subject into an intervention recommendation model to generate a medical intervention recommendation for the subject, wherein the intervention recommendation model is stored by a primary system, the primary system in communication with one or more third-party systems remote from the primary system, and wherein the intervention recommendation model comprises: a plurality of parameters identified by: providing the intervention recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model.

Additionally disclosed herein is a method for determining a medical intervention recommendation for a subject diagnosed with a condition, the method comprising the steps of: obtaining electronic health record data for the subject; automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject; inputting, using a computer processor, the electronic health record data and the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model.

Additionally disclosed herein is a method, comprising: determining a medical intervention recommendation for a subject diagnosed with a condition by: obtaining electronic health record data for the subject; obtaining biomarker data for the subject, the biomarker data obtained from a sample from the subject; inputting, using a computer processor, the electronic health record data and the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model; and generating a dataset that provides evidence in support of an indication for a medical intervention recommendation for the condition, the medical intervention recommendation determined by the intervention recommendation model using electronic health record data and biomarker data for one or more subjects diagnosed with the condition, the indication comprising values for at least one of electronic health record data and biomarker data used by the intervention recommendation model to determine the medical intervention recommendation for one or more subjects and based on a medical outcome of the one or more subjects.

Additionally disclosed herein is a method for determining a medical intervention recommendation for a subject diagnosed with a condition, the method comprising the steps of: obtaining electronic health record data for the subject; obtaining biomarker data for the subject, the biomarker data obtained from a sample from the subject; inputting, using a computer processor, the electronic health record data and the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model, wherein the medical intervention recommendation for the subject output by the intervention recommendation model fulfills at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, improved patient satisfaction with a patient care center at which the subject receives the medical intervention recommendation, increased patient throughput at a patient care center at which the subject receives the medical intervention recommendation, and increased revenue of a patient care center at which the subject receives the medical intervention recommendation.

In various embodiments, prior to inputting the electronic health record data and the biomarker data for the subject into the intervention recommendation model, transforming the electronic heath record data and the biomarker data into a common data format. In various embodiments, each training sample of the training dataset further comprises: a medical intervention provided to the retrospective subject associated with the training sample; and a medical outcome of the retrospective subject following receipt of the medical intervention recommendation. In various embodiments, the intervention recommendation model is stored by a primary system, the primary system in communication with one or more third-party systems. In various embodiments, the one or more third-party systems are remote from the primary system. In various embodiments, the one or more third-party systems are located at one or more patient care centers.

In various embodiments, the method further comprises receiving, from the one or more third-party systems, at the primary system, one or more of the plurality of training samples of the training dataset; and identifying, at the primary system, the plurality of parameters using the plurality of training samples received from the one or more third-party systems, wherein obtaining the electronic health record data and the biomarker data for the subject comprises receiving the electronic health record data and the biomarker data for the subject from the one or more third-party systems at the primary system, and wherein the medical intervention recommendation generated for the subject by the intervention recommendation model is generated at the primary system using the electronic health record data and the biomarker data for the subject. In various embodiments, the method further comprises receiving, from the one or more third-party systems, at the primary system, one or more of the plurality of training samples of the training dataset; identifying, at the primary system, the plurality of parameters using the plurality of training samples received from the one or more third-party systems; and providing the intervention recommendation model to the one or more third-party systems via network transmission, wherein obtaining the electronic health record data and the biomarker data for the subject comprises receiving the electronic health record data and the biomarker data for the subject at the intervention recommendation model at the one or more third-party systems, and wherein the medical intervention recommendation generated for the subject by the intervention recommendation model is generated at the one or more third-party systems using the electronic health record data and the biomarker data for the subject. In various embodiments, providing the intervention recommendation model to the one or more third-party systems comprises automatically providing the intervention recommendation model to the one or more third-party systems at specified time intervals.

In various embodiments, providing the intervention recommendation model to the one or more third-party systems comprises automatically providing the intervention recommendation model to the one or more third-party systems in real-time, near real-time, delayed batch or on-demand following identification of the plurality of parameters. In various embodiments, the method further comprises: providing the intervention recommendation model to the one or more third-party systems via network transmission; receiving one or more of the plurality of training samples of the training dataset at the intervention recommendation model at the one or more third-party systems; identifying, at the one or more third-party systems, the plurality of parameters using the training samples received at the intervention recommendation model at the one or more third-party systems; receiving the intervention recommendation model with the identified plurality of parameters at the primary system via network transmission, wherein obtaining the electronic health record data and the biomarker data for the subject comprises receiving the electronic health record data and the biomarker data for the subject from the one or more third-party systems at the primary system, and wherein the medical intervention recommendation generated for the subject by the intervention recommendation model is generated at the primary system using the electronic health record data and the biomarker data for the subject.

In various embodiments, receiving the intervention recommendation model with the identified plurality of parameters at the primary system comprises automatically receiving the intervention recommendation model with the identified plurality of parameters at the primary system at specified time intervals. In various embodiments, receiving the intervention recommendation model with the identified plurality of parameters at the primary system comprises automatically receiving the intervention recommendation model with the identified plurality of parameters at the primary system in real-time, near real-time, delayed batch or on-demand following identification of the plurality of parameters.

In various embodiments, the method further comprises providing the intervention recommendation model to the one or more third-party systems via network transmission; receiving one or more of the plurality of training samples of the training dataset at the intervention recommendation model at the one or more third-party systems; identifying, at the one or more third-party systems, the plurality of parameters using the training samples received at the intervention recommendation model at the one or more third-party systems; wherein obtaining the electronic health record data and the biomarker data for the subject comprises receiving the electronic health record data and the biomarker data for the subject at the intervention recommendation model at the one or more third-party systems, and wherein the medical intervention recommendation generated for the subject by the intervention recommendation model is generated at the one or more third-party systems using the electronic health record data and the biomarker data for the subject.

In various embodiments, the plurality of training samples are received from the one or more third-party systems at the primary system via network transmission. In various embodiments, the one or more of the plurality of training samples are received from multiple distinct third-party systems and comprise different data formats, and wherein the method further comprises: transforming the one or more of the plurality of training samples received from the multiple distinct third-party systems into a common data format; and merging the transformed training samples in a merged training dataset, wherein identifying the plurality of parameters using the plurality of training samples received from the one or more third-party systems comprises identifying the plurality of parameters using the merged training dataset. In various embodiments, the one or more of the plurality of training samples received from the multiple distinct third-party systems are transformed into the common data format using a publicly-available data transformation model. In various embodiments, the one or more of the plurality of training samples are received at the intervention recommendation model at multiple distinct third-party systems.

In various embodiments, the electronic health record data and the biomarker data for the subject is received from the one or more third-party systems at the primary system via network transmission. In various embodiments, returning the medical intervention recommendation for the subject output by the intervention recommendation model comprises providing the medical intervention recommendation for the subject to the one or more third-party systems via network transmission. In various embodiments, returning the medical intervention recommendation for the subject output by the intervention recommendation model comprises providing the medical intervention recommendation to the subject. In various embodiments, the one or more of the plurality of training samples are automatically received at specified time intervals and the plurality of parameters are automatically identified using the received training samples at specified time intervals, such that the intervention recommendation model is automatically updated at specified time intervals. In various embodiments, the one or more of the plurality of training samples are automatically received in real-time, near real-time, delayed batch or on-demand and the plurality of parameters are automatically identified in-real time using the received training samples. such that the intervention recommendation model is automatically updated in-real time.

In various embodiments, the method further comprises: generating a dataset that provides evidence in support of an indication for a medical intervention recommendation for the condition, the medical intervention recommendation determined by the intervention recommendation model using electronic health record data and biomarker data for one or more subjects diagnosed with the condition, the indication comprising values for at least one of electronic health record data and biomarker data used by the intervention recommendation model to determine the medical intervention recommendation for one or more subjects and based on a medical outcome of the one or more subjects. In various embodiments, at least one of the electronic health record data and the biomarker data are at least one of publicly-available data and commercially-available data. In various embodiments, at least one of the electronic health record data and the biomarker data for the subject or the retrospective subject are retrospective data. In various embodiments, at least one of the electronic health record data and the biomarker data for the subject are prospective data. In various embodiments, the electronic health record data is obtained from a patient care center. In various embodiments, the electronic health record data is obtained from a laboratory. In various embodiments, the biomarker data is obtained from the sample from the subject using a CLIA-certified laboratory.

In various embodiments, the biomarker data is obtained from the sample from the subject using an in vitro diagnostic device. In various embodiments, obtaining the biomarker data from the sample from the subject comprises receiving un-processed data directly from the in vitro diagnostic device. In various embodiments, the biomarker data is obtained from the sample from the subject on-site at a patient care center where the subject is located. In various embodiments, the biomarker data is obtained from the sample from the subject off-site from a patient care center where the subject is located. In various embodiments, the sample from the subject comprises a blood sample. In various embodiments, the sample from the subject comprises a urine sample. In various embodiments, the sample from the subject comprises a sample collected with one or more of a FDA-cleared, commercially-available sample collection, transport, and processing device.

In various embodiments, obtaining biomarker data for the subject comprises obtaining, from the sample from the subject, at least one of mass spectrometry, immunoassay, exome, transcriptome, or whole genome nucleotide sequencing data for the subject. In various embodiments, obtaining biomarker data for the subject comprises obtaining, from the sample from the subject, proteome data for the subject. In various embodiments, obtaining biomarker data for the subject comprises obtaining, from the sample from the subject, metabolome data for the subject. In various embodiments, obtaining biomarker data for the subject comprises obtaining, from the sample from the subject, lipidome data for the subject. In various embodiments, biomarker data for the subject comprises a quantification of expression of each of a plurality of genes in a gene panel. In various embodiments, the determined medical intervention recommendation is at least one of a selection, dosage, timing, starting, stopping, and monitoring of one or more pharmaceutical compounds, drugs, and biologics. In various embodiments, the determined medical intervention recommendation is a non-pharmaceutical intervention. In various embodiments, the medical intervention recommendation for the subject output by the intervention recommendation model fulfills at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, increased patient throughput at a patient care center at which the subject receives the medical intervention recommendation, and increased revenue of a patient care center at which the subject receives the medical intervention recommendation.

In various embodiments, the condition comprises one of sepsis, septic shock, refractory septic shock, acute lung injury, acute respiratory distress syndrome, acute renal failure, acute kidney injury, trauma, burns, COVID19, pneumonia, viral infection, and post-operative conditions. In various embodiments, the intervention recommendation model is a machine-learned model. In various embodiments, the plurality of parameters of the intervention recommendation model are identified using the training dataset by implementing federated learning. In various embodiments, inputting, using the computer processor, the electronic health record data and the biomarker data for the subject into an intervention recommendation model comprises monitoring computational operations for satisfying a computational metric. In various embodiments, responsive to monitoring that the computational metric is satisfied, scaling up or scaling down computational operations. In various embodiments, the computational metric is one or more of CPU utilization exceeding or falling below a threshold value, memory utilization exceeding or falling below a specified value, number of TCP connections exceeding or falling below a specified value, number of pending computational messages exceeding or falling below a specified value.

Additionally disclosed herein is a method for determining a medical diagnosis recommendation of a subject, the method comprising the steps of: obtaining electronic health record data for the subject; obtaining biomarker data for the subject, the biomarker data obtained from a sample from the subject; inputting, using a computer processor, the electronic health record data and the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and returning the medical diagnosis recommendation for the subject output by the diagnostic recommendation model.

Additionally disclosed herein is a method for determining a medical diagnosis recommendation of a subject, the method comprising the steps of: obtaining electronic health record data for the subject; obtaining biomarker data for the subject, the biomarker data obtained from a sample from the subject; inputting, using a computer processor, the electronic health record data and the biomarker data for the subject into a diagnostic recommendation model to generate a medical diagnosis recommendation for the subject, wherein the diagnostic recommendation model is stored by a primary system, the primary system in communication with one or more third-party systems remote from the primary system, and wherein the diagnostic recommendation model comprises: a plurality of parameters identified by: providing the diagnostic recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and returning the medical diagnosis recommendation for the subject output by the diagnostic recommendation model.

Additionally disclosed herein is a method for determining a medical diagnosis recommendation of a subject, the method comprising the steps of: obtaining electronic health record data for the subject; automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject; inputting, using a computer processor, the electronic health record data and the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and returning the medical diagnosis recommendation for the subject output by the diagnostic recommendation model.

Additionally disclosed herein is a method for determining a medical diagnosis recommendation of a subject, the method comprising the steps of: obtaining electronic health record data for the subject; obtaining biomarker data for the subject, the biomarker data obtained from a sample from the subject; inputting, using a computer processor, the electronic health record data and the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and returning the medical diagnosis recommendation for the subject output by the diagnostic recommendation model, wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

In various embodiments, each training sample of the training dataset further comprises: a medical diagnosis of the retrospective subject associated with the training sample; and a medical outcome of the retrospective subject following receipt of the medical diagnosis. In various embodiments, the diagnostic recommendation model is stored by a primary system, the primary system in communication with one or more third-party systems. In various embodiments, the one or more third-party systems are remote from the primary system. In various embodiments, the one or more third-party systems are located at one or more patient care centers. In various embodiments, the method further comprises: receiving, from the one or more third-party systems, at the primary system, one or more of the plurality of training samples of the training dataset; and identifying, at the primary system, the plurality of parameters using the plurality of training samples received from the one or more third-party systems, wherein obtaining the electronic health record data and the biomarker data for the subject comprises receiving the electronic health record data and the biomarker data for the subject from the one or more third-party systems at the primary system, and wherein the medical diagnosis recommendation generated for the subject by the diagnostic recommendation model is generated at the primary system using the electronic health record data and the biomarker data for the subject.

In various embodiments, the method further comprises; receiving, from the one or more third-party systems, at the primary system, one or more of the plurality of training samples of the training dataset; identifying, at the primary system, the plurality of parameters using the plurality of training samples received from the one or more third-party systems; and providing the diagnostic recommendation model to the one or more third-party systems via network transmission, wherein obtaining the electronic health record data and the biomarker data for the subject comprises receiving the electronic health record data and the biomarker data for the subject at the diagnostic recommendation model at the one or more third-party systems, and wherein the medical diagnosis recommendation generated for the subject by the diagnostic recommendation model is generated at the one or more third-party systems using the electronic health record data and the biomarker data for the subject. In various embodiments, providing the diagnostic recommendation model to the one or more third-party systems comprises automatically providing the diagnostic recommendation model to the one or more third-party systems at specified time intervals. In various embodiments, providing the diagnostic recommendation model to the one or more third-party systems comprises automatically providing the diagnostic recommendation model to the one or more third-party systems in real-time, near real-time, delayed batch or on-demand following identification of the plurality of parameters.

In various embodiments, the method further comprises: providing the diagnostic recommendation model to the one or more third-party systems via network transmission; receiving one or more of the plurality of training samples of the training dataset at the diagnostic recommendation model at the one or more third-party systems; identifying, at the one or more third-party systems, the plurality of parameters using the training samples received at the diagnostic recommendation model at the one or more third-party systems; receiving the diagnostic recommendation model with the identified plurality of parameters at the primary system via network transmission, wherein obtaining the electronic health record data and the biomarker data for the subject comprises receiving the electronic health record data and the biomarker data for the subject from the one or more third-party systems at the primary system, and wherein the medical diagnosis recommendation generated for the subject by the diagnostic recommendation model is generated at the primary system using the electronic health record data and the biomarker data for the subject. In various embodiments, receiving the diagnostic recommendation model with the identified plurality of parameters at the primary system comprises automatically receiving the diagnostic recommendation model with the identified plurality of parameters at the primary system at specified time intervals. In various embodiments, receiving the diagnostic recommendation model with the identified plurality of parameters at the primary system comprises automatically receiving the diagnostic recommendation model with the identified plurality of parameters at the primary system in real-time, near real-time, delayed batch or on-demand following identification of the plurality of parameters. In various embodiments, the method further comprises: providing the diagnostic recommendation model to the one or more third-party systems via network transmission; receiving one or more of the plurality of training samples of the training dataset at the diagnostic recommendation model at the one or more third-party systems; identifying, at the one or more third-party systems, the plurality of parameters using the training samples received at the diagnostic recommendation model at the one or more third-party systems; wherein obtaining the electronic health record data and the biomarker data for the subject comprises receiving the electronic health record data and the biomarker data for the subject at the diagnostic recommendation model at the one or more third-party systems, and wherein the medical diagnosis recommendation generated for the subject by the diagnostic recommendation model is generated at the one or more third-party systems using the electronic health record data and the biomarker data for the subject. In various embodiments, the plurality of training samples are received from the one or more third-party systems at the primary system via network transmission. In various embodiments, the one or more of the plurality of training samples are received from multiple distinct third-party systems and comprise different data formats, and wherein the method further comprises: transforming the one or more of the plurality of training samples received from the multiple distinct third-party systems into a common data format; and merging the transformed training samples in a merged training dataset, wherein identifying the plurality of parameters using the plurality of training samples received from the one or more third-party systems comprises identifying the plurality of parameters using the merged training dataset.

In various embodiments, one or more of the plurality of training samples received from the multiple distinct third-party systems are transformed into the common data format using a publicly-available data transformation model. In various embodiments, the one or more of the plurality of training samples are received at the diagnostic recommendation model at multiple distinct third-party systems. In various embodiments, the electronic health record data and the biomarker data for the subject is received from the one or more third-party systems at the primary system via network transmission. In various embodiments, returning the diagnosis for the subject output by the diagnostic recommendation model comprises providing the medical diagnosis recommendation for the subject to the one or more third-party systems via network transmission. In various embodiments, the one or more of the plurality of training samples are automatically received at specified time intervals and the plurality of parameters are automatically identified using the received training samples at specified time intervals, such that the diagnostic recommendation model is automatically updated at specified time intervals. In various embodiments, the one or more of the plurality of training samples are automatically received in real-time and the plurality of parameters are automatically identified in-real time using the received training samples. such that the diagnostic recommendation model is automatically updated in-real time.

In various embodiments, at least one of the electronic health record data and the biomarker data are at least one of publicly-available data and commercially-available data. In various embodiments, at least one of the electronic health record data and the biomarker data for the subject or the retrospective subject are retrospective data. In various embodiments, at least one of the electronic health record data and the biomarker data for the subject are prospective data. In various embodiments, the electronic health record data is obtained from a patient care center. In various embodiments, the electronic health record data is obtained from a laboratory. In various embodiments, the biomarker data is obtained from the sample from the subject using a CLIA-certified laboratory. In various embodiments, the biomarker data is obtained from the sample from the subject using an in vitro diagnostic device. In various embodiments, obtaining the biomarker data from the sample from the subject comprises receiving un-processed data directly from the in vitro diagnostic device.

In various embodiments, the biomarker data is obtained from the sample from the subject on-site at a patient care center where the subject is located. In various embodiments, the biomarker data is obtained from the sample from the subject off-site from a patient care center where the subject is located. In various embodiments, the sample from the subject comprises a blood sample. In various embodiments, the sample from the subject comprises a urine sample. In various embodiments, the sample from the subject comprises a sample collected with one or more of a FDA-cleared, commercially-available sample collection, transport, and processing device. In various embodiments, obtaining biomarker data for the subject comprises obtaining, from the sample from the subject, at least one of mass spectrometry, immunoassay, exome, transcriptome, or whole genome nucleotide sequencing data for the subject. In various embodiments, obtaining biomarker data for the subject comprises obtaining, from the sample from the subject, proteome data for the subject. In various embodiments, obtaining biomarker data for the subject comprises obtaining, from the sample from the subject, metabolome data for the subject. In various embodiments, obtaining biomarker data for the subject comprises obtaining, from the sample from the subject, lipidome data for the subject. In various embodiments, biomarker data for the subject comprises a quantification of expression of each of a plurality of genes in a gene panel. In various embodiments, the method further comprises providing a medical intervention recommendation to the subject based on the determined medical diagnosis recommendation, the medical intervention recommendation comprising at least one of a selection, dosage, timing, starting, stopping, and monitoring of one or more pharmaceutical compounds, drugs, and biologics. In various embodiments, the method further comprises providing a medical intervention recommendation to the subject based on the determined medical diagnosis recommendation, the medical intervention recommendation comprising a non-pharmaceutical intervention.

In various embodiments, the medical diagnosis recommendation for the subject output by the diagnostic recommendation model fulfills at least one of the following when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

In various embodiments, the determined medical diagnosis recommendation of the subject comprises one of sepsis, septic shock, refractory septic shock, acute lung injury, acute respiratory distress syndrome, acute renal failure, acute kidney injury, trauma, burns, COVID19, pneumonia, viral infection, and post-operative conditions. In various embodiments, the diagnostic recommendation model is a machine-learned model. In various embodiments, the plurality of parameters of the diagnostic recommendation model are identified using the training dataset by implementing federated learning. In various embodiments, inputting, using the computer processor, the electronic health record data and the biomarker data for the subject into an diagnostic recommendation model comprises monitoring computational operations for satisfying a computational metric. In various embodiments, responsive to monitoring that the computational metric is satisfied, scaling up or scaling down computational operations. In various embodiments, the computational metric is one or more of CPU utilization exceeding or falling below a threshold value, memory utilization exceeding or falling below a specified value, number of TCP connections exceeding or falling below a specified value, number of pending computational messages exceeding or falling below a specified value.

Additionally disclosed herein is a non-transitory computer-readable storage medium storing computer program instructions that when executed by a computer processor, cause the computer processor to determine a medical intervention recommendation for a subject diagnosed with a condition by: obtaining electronic health record data for the subject; obtaining biomarker data for the subject, the biomarker data obtained from a sample from the subject; inputting, using the computer processor, the electronic health record data and the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model.

Additionally disclosed herein is a non-transitory computer-readable storage medium storing computer program instructions that when executed by a computer processor, cause the computer processor to determine a medical intervention recommendation for a subject diagnosed with a condition by: obtaining electronic health record data for the subject; obtaining biomarker data for the subject, the biomarker data obtained from a sample from the subject; inputting, using the computer processor, the electronic health record data and the biomarker data for the subject into an intervention recommendation model to generate a medical intervention recommendation for the subject, wherein the intervention recommendation model is stored by a primary system, the primary system in communication with one or more third-party systems remote from the primary system, and wherein the intervention recommendation model comprises: a plurality of parameters identified by: providing the intervention recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model.

Additionally disclosed herein is a non-transitory computer-readable storage medium storing computer program instructions that when executed by a computer processor, cause the computer processor to determine a medical intervention recommendation for a subject diagnosed with a condition by: obtaining electronic health record data for the subject; automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject; inputting, using the computer processor, the electronic health record data and the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model.

Additionally disclosed herein is a non-transitory computer-readable storage medium storing computer program instructions that when executed by a computer processor, cause the computer processor to: determine a medical intervention recommendation for a subject diagnosed with a condition by: obtaining electronic health record data for the subject; obtaining biomarker data for the subject, the biomarker data obtained from a sample from the subject; inputting, using the computer processor, the electronic health record data and the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model; and generating a dataset that provides evidence in support of an indication for a medical intervention recommendation for the condition, the medical intervention recommendation determined by the intervention recommendation model using electronic health record data and biomarker data for one or more subjects diagnosed with the condition, the indication comprising values for at least one of electronic health record data and biomarker data used by the intervention recommendation model to determine the medical intervention recommendation for one or more subjects and based on a medical outcome of the one or more subjects.

Additionally disclosed herein is a non-transitory computer-readable storage medium storing computer program instructions that when executed by a computer processor, cause the computer processor to determine a medical intervention recommendation for a subject diagnosed with a condition by: obtaining electronic health record data for the subject; obtaining biomarker data for the subject, the biomarker data obtained from a sample from the subject; inputting, using a computer processor, the electronic health record data and the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and returning the medical intervention recommendation for the subject output by the intervention recommendation model, wherein the medical intervention recommendation for the subject output by the intervention recommendation model fulfills at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, increased patient throughput at a patient care center at which the subject receives the medical intervention recommendation, and increased revenue of a patient care center at which the subject receives the medical intervention recommendation.

Additionally disclosed herein is a non-transitory computer-readable storage medium storing computer program instructions that when executed by a computer processor, cause the computer processor to determine a medical diagnosis recommendation of a subject by: obtaining electronic health record data for the subject; obtaining biomarker data for the subject, the biomarker data obtained from a sample from the subject; inputting, using the computer processor, the electronic health record data and the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and returning the medical diagnosis recommendation for the subject output by the diagnostic recommendation model.

Additionally disclosed herein is a non-transitory computer-readable storage medium storing computer program instructions that when executed by a computer processor, cause the computer processor to determine a medical diagnosis recommendation of a subject by: obtaining electronic health record data for the subject; obtaining biomarker data for the subject, the biomarker data obtained from a sample from the subject; inputting, using the computer processor, the electronic health record data and the biomarker data for the subject into a diagnostic recommendation model to generate a medical diagnosis recommendation for the subject, wherein the diagnostic recommendation model is stored by the non-transitory computer-readable storage medium, the non-transitory computer-readable storage medium in communication with one or more third-party systems remote from the non-transitory computer-readable storage medium, and wherein the diagnostic recommendation model comprises: a plurality of parameters identified by: providing the diagnostic recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and returning the medical diagnosis recommendation for the subject output by the diagnostic recommendation model.

Additionally disclosed herein is a non-transitory computer-readable storage medium storing computer program instructions that when executed by a computer processor, cause the computer processor to determine a medical diagnosis recommendation of a subject by: obtaining electronic health record data for the subject; automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject; inputting, using the computer processor, the electronic health record data and the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and returning the medical diagnosis recommendation for the subject output by the diagnostic recommendation model.

Additionally disclosed herein is a non-transitory computer-readable storage medium storing computer program instructions that when executed by a computer processor, cause the computer processor to determine a medical diagnosis recommendation of a subject by: obtaining electronic health record data for the subject; obtaining biomarker data for the subject, the biomarker data obtained from a sample from the subject; inputting, using the computer processor, the electronic health record data and the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and biomarker data for the retrospective subject, the biomarker data obtained from a sample from the retrospective subject; and a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and returning the medical diagnosis recommendation for the subject output by the diagnostic recommendation model, wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

Additionally disclosed herein is a method for determining a medical intervention recommendation for a subject diagnosed with a condition, the method comprising the steps of: obtaining electronic health record data for the subject; inputting, using a computer processor, the electronic health record data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and returning the medical intervention recommendation for the subject output by the intervention recommendation model.

Additionally disclosed herein is a method for determining a medical intervention recommendation for a subject diagnosed with a condition, the method comprising the steps of: obtaining electronic health record data for the subject; inputting, using a computer processor, the electronic health record data for the subject into an intervention recommendation model to generate a medical intervention recommendation for the subject, wherein the intervention recommendation model is stored by a primary system, the primary system in communication with one or more third-party systems remote from the primary system, and wherein the intervention recommendation model comprises: a plurality of parameters identified by: providing the intervention recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: electronic health record data for the retrospective subject; and a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and returning the medical intervention recommendation for the subject output by the intervention recommendation model.

Additionally disclosed herein is a method comprising: determining a medical intervention recommendation for a subject diagnosed with a condition by: obtaining electronic health record data for the subject; inputting, using a computer processor, the electronic health record data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and returning the medical intervention recommendation for the subject output by the intervention recommendation model; and generating a dataset that provides evidence in support of an indication for a medical intervention recommendation for the condition, the medical intervention recommendation determined by the intervention recommendation model using electronic health record data for one or more subjects diagnosed with the condition, the indication comprising values for electronic health record data used by the intervention recommendation model to determine the medical intervention recommendation for one or more subjects and based on a medical outcome of the one or more subjects.

Additionally disclosed herein is a method for determining a medical intervention recommendation for a subject diagnosed with a condition, the method comprising the steps of: obtaining electronic health record data for the subject; inputting, using a computer processor, the electronic health record data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and returning the medical intervention recommendation for the subject output by the intervention recommendation model, wherein the medical intervention recommendation for the subject output by the intervention recommendation model fulfills at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, increased patient throughput at a patient care center at which the subject receives the medical intervention recommendation, and increased revenue of a patient care center at which the subject receives the medical intervention recommendation.

Additionally disclosed herein is a method for determining a medical diagnosis recommendation of a subject, the method comprising the steps of: obtaining electronic health record data for the subject; inputting, using a computer processor, the electronic health record data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and returning the medical diagnosis recommendation for the subject output by the diagnostic recommendation model.

Additionally disclosed herein is a method for determining a medical diagnosis recommendation of a subject, the method comprising the steps of: obtaining electronic health record data for the subject; inputting, using a computer processor, the electronic health record data for the subject into a diagnostic recommendation model to generate a medical diagnosis recommendation for the subject, wherein the diagnostic recommendation model is stored by a primary system, the primary system in communication with one or more third-party systems remote from the primary system, and wherein the diagnostic recommendation model comprises: a plurality of parameters identified by: providing the diagnostic recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: electronic health record data for the retrospective subject; and a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and returning the medical diagnosis recommendation for the subject output by the diagnostic recommendation model.

Additionally disclosed herein is a method for determining a medical diagnosis recommendation of a subject, the method comprising the steps of: obtaining electronic health record data for the subject; inputting, using a computer processor, the electronic health record data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and returning the medical diagnosis recommendation for the subject output by the diagnostic recommendation model, wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

Additionally disclosed herein is a non-transitory computer-readable storage medium storing computer program instructions that when executed by a computer processor, cause the computer processor to determine a medical intervention recommendation for a subject diagnosed with a condition by: obtaining electronic health record data for the subject; inputting, using the computer processor, the electronic health record data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and returning the medical intervention recommendation for the subject output by the intervention recommendation model.

Additionally disclosed herein is a non-transitory computer-readable storage medium storing computer program instructions that when executed by a computer processor, cause the computer processor to determine a medical intervention recommendation for a subject diagnosed with a condition by: obtaining electronic health record data for the subject; inputting, using the computer processor, the electronic health record data for the subject into an intervention recommendation model to generate a medical intervention recommendation for the subject, wherein the intervention recommendation model is stored by a primary system, the primary system in communication with one or more third-party systems remote from the primary system, and wherein the intervention recommendation model comprises: a plurality of parameters identified by: providing the intervention recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: electronic health record data for the retrospective subject; and a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and returning the medical intervention recommendation for the subject output by the intervention recommendation model.

Additionally disclosed herein is a non-transitory computer-readable storage medium storing computer program instructions that when executed by a computer processor, cause the computer processor to: determine a medical intervention recommendation for a subject diagnosed with a condition by: obtaining electronic health record data for the subject; inputting, using the computer processor, the electronic health record data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and returning the medical intervention recommendation for the subject output by the intervention recommendation model; and generating a dataset that provides evidence in support of an indication for a medical intervention recommendation for the condition, the medical intervention recommendation determined by the intervention recommendation model using electronic health record data for one or more subjects diagnosed with the condition, the indication comprising values for electronic health record data used by the intervention recommendation model to determine the medical intervention recommendation for one or more subjects and based on a medical outcome of the one or more subjects.

Additionally disclosed herein is a non-transitory computer-readable storage medium storing computer program instructions that when executed by a computer processor, cause the computer processor to determine a medical intervention recommendation for a subject diagnosed with a condition by: obtaining electronic health record data for the subject; inputting, using a computer processor, the electronic health record data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and returning the medical intervention recommendation for the subject output by the intervention recommendation model, wherein the medical intervention recommendation for the subject output by the intervention recommendation model fulfills at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, increased patient throughput at a patient care center at which the subject receives the medical intervention recommendation, and increased revenue of a patient care center at which the subject receives the medical intervention recommendation.

Additionally disclosed herein is a non-transitory computer-readable storage medium storing computer program instructions that when executed by a computer processor, cause the computer processor to determine a medical diagnosis recommendation of a subject by: obtaining electronic health record data for the subject; inputting, using the computer processor, the electronic health record data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and returning the medical diagnosis recommendation for the subject output by the diagnostic recommendation model.

Additionally disclosed herein is a non-transitory computer-readable storage medium storing computer program instructions that when executed by a computer processor, cause the computer processor to determine a medical diagnosis recommendation of a subject by: obtaining electronic health record data for the subject; inputting, using the computer processor, the electronic health record data for the subject into a diagnostic recommendation model to generate a medical diagnosis recommendation for the subject, wherein the diagnostic recommendation model is stored by the non-transitory computer-readable storage medium, the non-transitory computer-readable storage medium in communication with one or more third-party systems remote from the non-transitory computer-readable storage medium, and wherein the diagnostic recommendation model comprises: a plurality of parameters identified by: providing the diagnostic recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: electronic health record data for the retrospective subject; and a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and returning the medical diagnosis recommendation for the subject output by the diagnostic recommendation model.

Additionally disclosed herein is a non-transitory computer-readable storage medium storing computer program instructions that when executed by a computer processor, cause the computer processor to determine a medical diagnosis recommendation of a subject by: obtaining electronic health record data for the subject; inputting, using the computer processor, the electronic health record data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: electronic health record data for the retrospective subject; and a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and returning the medical diagnosis recommendation for the subject output by the diagnostic recommendation model, wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

Additionally disclosed herein is a method for determining a medical intervention recommendation for a subject diagnosed with a condition, the method comprising the steps of: obtaining biomarker data for the subject, the biomarker data obtained from a sample from the subject; inputting, using a computer processor, the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model.

Additionally disclosed herein is a method for determining a medical intervention recommendation for a subject diagnosed with a condition, the method comprising the steps of: obtaining biomarker data for the subject, the biomarker data obtained from a sample from the subject; inputting, using a computer processor, the biomarker data for the subject into an intervention recommendation model to generate a medical intervention recommendation for the subject, wherein the intervention recommendation model is stored by a primary system, the primary system in communication with one or more third-party systems remote from the primary system, and wherein the intervention recommendation model comprises: a plurality of parameters identified by: providing the intervention recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model.

Additionally disclosed herein is a method for determining a medical intervention recommendation for a subject diagnosed with a condition, the method comprising the steps of: automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject; inputting, using a computer processor, the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model.

Additionally disclosed herein is a method comprising: determining a medical intervention recommendation for a subject diagnosed with a condition by: obtaining biomarker data for the subject, the biomarker data obtained from a sample from the subject; inputting, using a computer processor, the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model; and generating a dataset that provides evidence in support of an indication for a medical intervention recommendation for the condition, the medical intervention recommendation determined by the intervention recommendation model using biomarker data for one or more subjects diagnosed with the condition, the indication comprising values for biomarker data used by the intervention recommendation model to determine the medical intervention recommendation for one or more subjects and based on a medical outcome of the one or more subjects.

Additionally disclosed herein is a method for determining a medical intervention recommendation for a subject diagnosed with a condition, the method comprising the steps of: obtaining biomarker data for the subject, the biomarker data obtained from a sample from the subject; inputting, using a computer processor, the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model, wherein the medical intervention recommendation for the subject output by the intervention recommendation model fulfills at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, increased patient throughput at a patient care center at which the subject receives the medical intervention recommendation, and increased revenue of a patient care center at which the subject receives the medical intervention recommendation.

Additionally disclosed herein is a method for determining a medical diagnosis recommendation of a subject, the method comprising the steps of: obtaining biomarker data for the subject, the biomarker data obtained from a sample from the subject; inputting, using a computer processor, the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and returning the medical diagnosis recommendation for the subject output by the diagnostic recommendation model.

Additionally disclosed herein is a method for determining a medical diagnosis recommendation of a subject, the method comprising the steps of: obtaining biomarker data for the subject, the biomarker data obtained from a sample from the subject; inputting, using a computer processor, the biomarker data for the subject into a diagnostic recommendation model to generate a medical diagnosis recommendation for the subject, wherein the diagnostic recommendation model is stored by a primary system, the primary system in communication with one or more third-party systems remote from the primary system, and wherein the diagnostic recommendation model comprises: a plurality of parameters identified by: providing the diagnostic recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and returning the medical diagnosis recommendation for the subject output by the diagnostic recommendation model.

Additionally disclosed herein is a method for determining a medical diagnosis recommendation of a subject, the method comprising the steps of: automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject; inputting, using a computer processor, the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and returning the medical diagnosis recommendation for the subject output by the diagnostic recommendation model.

Additionally disclosed herein is a method for determining a medical diagnosis recommendation of a subject, the method comprising the steps of: obtaining biomarker data for the subject, the biomarker data obtained from a sample from the subject; inputting, using a computer processor, the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and returning the medical diagnosis recommendation for the subject output by the diagnostic recommendation model, wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

Additionally disclosed herein is a non-transitory computer-readable storage medium storing computer program instructions that when executed by a computer processor, cause the computer processor to determine a medical intervention recommendation for a subject diagnosed with a condition by: obtaining biomarker data for the subject, the biomarker data obtained from a sample from the subject; inputting, using the computer processor, the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model.

Additionally disclosed herein is a non-transitory computer-readable storage medium storing computer program instructions that when executed by a computer processor, cause the computer processor to determine a medical intervention recommendation for a subject diagnosed with a condition by: obtaining biomarker data for the subject, the biomarker data obtained from a sample from the subject; inputting, using the computer processor, the biomarker data for the subject into an intervention recommendation model to generate a medical intervention recommendation for the subject, wherein the intervention recommendation model is stored by a primary system, the primary system in communication with one or more third-party systems remote from the primary system, and wherein the intervention recommendation model comprises: a plurality of parameters identified by: providing the intervention recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model.

Additionally disclosed herein is a non-transitory computer-readable storage medium storing computer program instructions that when executed by a computer processor, cause the computer processor to determine a medical intervention recommendation for a subject diagnosed with a condition by: automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject; inputting, using the computer processor, the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model.

Additionally disclosed herein is a non-transitory computer-readable storage medium storing computer program instructions that when executed by a computer processor, cause the computer processor to: determine a medical intervention recommendation for a subject diagnosed with a condition by: obtaining biomarker data for the subject, the biomarker data obtained from a sample from the subject; inputting, using the computer processor, the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model; and generating a dataset that provides evidence in support of an indication for a medical intervention recommendation for the condition, the medical intervention recommendation determined by the intervention recommendation model using biomarker data for one or more subjects diagnosed with the condition, the indication comprising values for biomarker data used by the intervention recommendation model to determine the medical intervention recommendation for one or more subjects and based on a medical outcome of the one or more subjects.

Additionally disclosed herein is a non-transitory computer-readable storage medium storing computer program instructions that when executed by a computer processor, cause the computer processor to determine a medical intervention recommendation for a subject diagnosed with a condition by: obtaining biomarker data for the subject, the biomarker data obtained from a sample from the subject; inputting, using a computer processor, the biomarker data for the subject into an intervention recommendation model to generate a medical intervention recommendation for the subject, the intervention recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and returning the medical intervention recommendation for the subject output by the intervention recommendation model, wherein the medical intervention recommendation for the subject output by the intervention recommendation model fulfills at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, increased patient throughput at a patient care center at which the subject receives the medical intervention recommendation, and increased revenue of a patient care center at which the subject receives the medical intervention recommendation.

Additionally disclosed herein is a non-transitory computer-readable storage medium storing computer program instructions that when executed by a computer processor, cause the computer processor to determine a medical diagnosis recommendation of a subject by: obtaining biomarker data for the subject, the biomarker data obtained from a sample from the subject; inputting, using the computer processor, the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and returning the medical diagnosis recommendation for the subject output by the diagnostic recommendation model.

Additionally disclosed herein is a non-transitory computer-readable storage medium storing computer program instructions that when executed by a computer processor, cause the computer processor to determine a medical diagnosis recommendation of a subject by: obtaining biomarker data for the subject, the biomarker data obtained from a sample from the subject; inputting, using the computer processor, the biomarker data for the subject into a diagnostic recommendation model to generate a medical diagnosis recommendation for the subject, wherein the diagnostic recommendation model is stored by the non-transitory computer-readable storage medium, the non-transitory computer-readable storage medium in communication with one or more third-party systems remote from the non-transitory computer-readable storage medium, and wherein the diagnostic recommendation model comprises: a plurality of parameters identified by: providing the diagnostic recommendation model to the one or more third-party systems via network transmission; identifying, at the one or more third-party systems, the plurality of parameters using a training dataset received at the one or more third-party systems, the training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and returning the medical diagnosis recommendation for the subject output by the diagnostic recommendation model.

Additionally disclosed herein is a non-transitory computer-readable storage medium storing computer program instructions that when executed by a computer processor, cause the computer processor to determine a medical diagnosis recommendation of a subject by: automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject; inputting, using the computer processor, the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and returning the medical diagnosis recommendation for the subject output by the diagnostic recommendation model.

Additionally disclosed herein is a non-transitory computer-readable storage medium storing computer program instructions that when executed by a computer processor, cause the computer processor to determine a medical diagnosis recommendation of a subject by: obtaining biomarker data for the subject, the biomarker data obtained from a sample from the subject; inputting, using the computer processor, the biomarker data for the subject into a diagnostic recommendation model to generate a medical diagnosis recommendation for the subject, the diagnostic recommendation model comprising: a plurality of parameters identified at least based on a training dataset comprising a plurality of training samples, each training sample associated with a retrospective subject and comprising: biomarker data for the retrospective subject, the biomarker data obtained from a sample from the retrospective subject; and a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and returning the medical diagnosis recommendation for the subject output by the diagnostic recommendation model, wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

### BRIEF DESCRIPTION OF THE DRAWINGS

These and other features, aspects, and advantages of the present invention will become better understood with regard to the following description, and accompanying drawings, where:

FIG. 1 is a block diagram of a system environment for a diagnostic/intervention recommendation system configured to determine at least one of a medical diagnosis recommendation for a subject and a medical intervention recommendation for a subject, in accordance with an embodiment.

FIG. 2 is a block diagram of a system environment for a diagnostic/intervention recommendation system configured to determine at least one of a medical diagnosis recommendation for a subject and a medical intervention recommendation for a subject using biomarker data automatically received from directly an in vitro diagnostic device, in accordance with an embodiment.

FIG. 3 is a block diagram of an architecture of a diagnostic/intervention recommendation system configured to determine at least one of a medical diagnosis recommendation for a subject and a medical intervention recommendation for a subject, in accordance with an embodiment.

FIG. 4 illustrates an example network model, in accordance with an embodiment.

FIG. 5A is a block diagram of a system environment in which a diagnostic/intervention recommendation system is trained, validated, and used, in accordance with an embodiment.

FIG. 5B is a block diagram of a system environment in which a diagnostic/intervention recommendation system is trained, in accordance with an embodiment.

FIG. 5C is a block diagram of a system environment in which the diagnostic/intervention recommendation system is validated, in accordance with an embodiment.

FIG. 5D is a block diagram of a system environment in which the diagnostic/intervention recommendation system is used, in accordance with an embodiment.

FIG. 6A is a block diagram of a system environment in which a diagnostic/intervention recommendation system operates, in accordance with an embodiment.

FIG. 6B is a block diagram of a system environment in which the diagnostic/intervention recommendation system operates, in accordance with an embodiment.

FIG. 6C is a block diagram of a system environment in which the diagnostic/intervention recommendation system operates, in accordance with an embodiment.

FIG. 6D is a block diagram of a system environment in which the diagnostic/intervention recommendation system operates, in accordance with an embodiment.

FIG. 6E is a block diagram of a system environment in which the diagnostic/intervention recommendation system operates, in accordance with an embodiment.

FIG. 7A depicts an example dataset that provides evidence in support of indications for corticosteroid intervention, in accordance with an embodiment.

FIG. 7B depicts an example dataset that provides evidence in support of indications for corticosteroid intervention, in accordance with an embodiment.

FIG. 8 is a flow chart of a method for determining a medical diagnosis/intervention recommendation for a subject, in accordance with an embodiment.

FIG. 9 illustrates an example computer for implementing the method described in FIG. 8, in accordance with an embodiment.

The figures depict various embodiments of the present invention for purposes of illustration only. One skilled in the art will readily recognize from the following discussion that alternative embodiments of the structures and methods illustrated herein can be employed without departing from the principles of the invention described herein.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### II. Diagnostic/Intervention Recommendation System

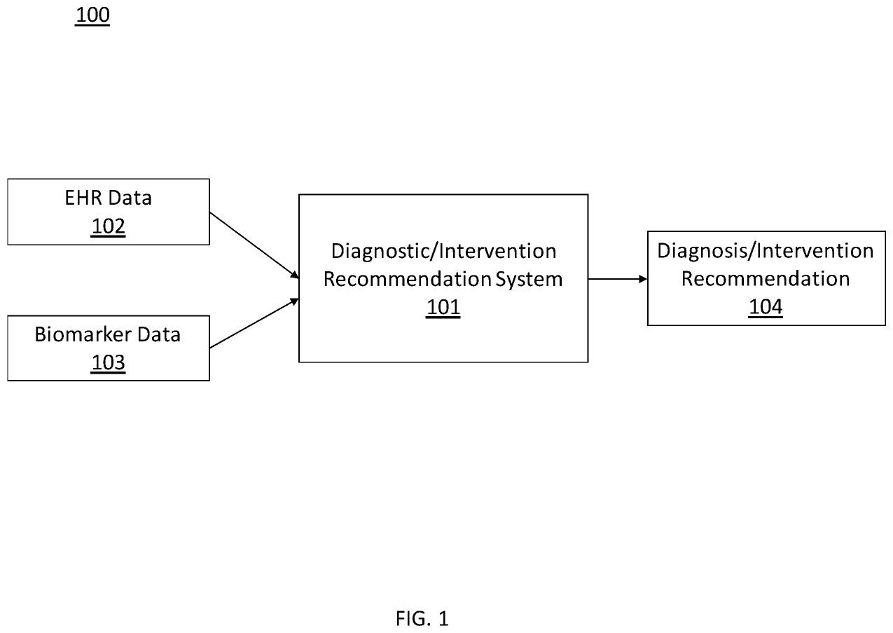

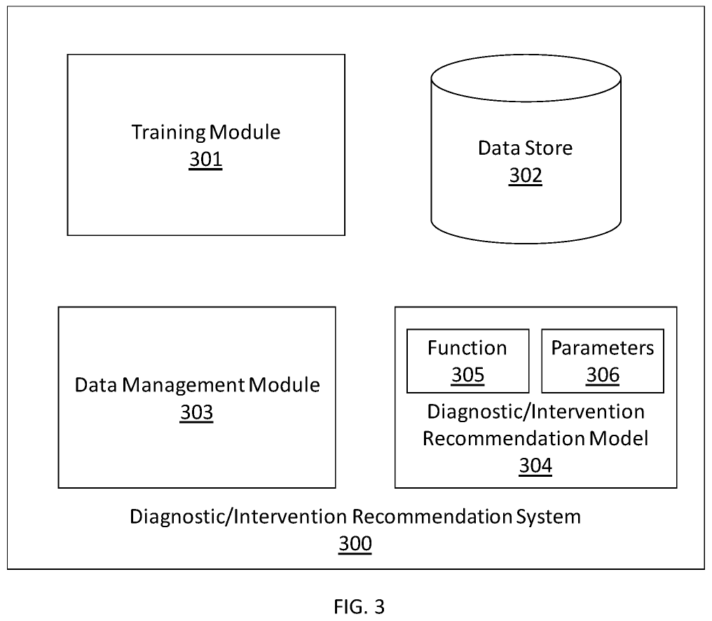

FIG. 1 is a block diagram of a system environment 100 for a diagnostic/intervention recommendation system 101 configured to determine at least one of a medical diagnosis recommendation for a subject and a medical intervention recommendation for a subject, in accordance with an embodiment. As discussed in further detail below with regard to FIG. 3, the diagnostic/intervention recommendation system 101 at least in part comprises a diagnostic/intervention recommendation model. In various embodiments, the diagnostic/intervention recommendation model is a machine-learned diagnostic/intervention recommendation model. In alternative configurations, different and/or additional components may be included in the system environment 100.

For simplicity, throughout this disclosure, the system and model discussed herein are referred to as a “diagnostic/intervention” recommendation system and model. In certain embodiments, the diagnostic/intervention recommendation system and model are configured to both recommend medical diagnoses for subjects, as well as recommend medical interventions for subjects. However, the nomenclature of “diagnostic/intervention” recommendation does not necessitate a single system or model configured to recommend both medical diagnoses and medical interventions for a subject. For instance, in some embodiments, the diagnostic/intervention recommendation system 101 can be strictly a diagnostic recommendation system or an intervention recommendation system. In alternative embodiments, the diagnostic/intervention recommendation system 101 can comprise two separate systems—the first system being a diagnostic recommendation system and the second system being an intervention recommendation system. In even further embodiments, the diagnostic/intervention recommendation system 101 can comprise more than two distinct systems. For example, in some embodiments, the diagnostic/intervention recommendation system 101 can comprise a plurality of distinct intervention recommendation systems, each associated with a specific condition and configured to determine an intervention recommendation for a subject diagnosed with the condition. As discussed in further detail below, the configuration of the diagnostic/intervention recommendation system 101 depends on the configuration of the diagnostic/intervention recommendation model, which at least partly comprises the diagnostic/intervention recommendation system 101.

The same alternative configuration embodiments hold true for the diagnostic/intervention recommendation model itself. Specifically, in certain embodiments, the diagnostic/intervention recommendation model is configured to both recommend medical diagnoses for subjects, as well as recommend medical interventions for subjects. Alternatively, in some embodiments, the diagnostic/intervention recommendation model is strictly a diagnostic recommendation model or an intervention recommendation model. In alternative embodiments, the diagnostic/intervention recommendation model comprises two separate models—the first being a diagnostic recommendation model and the second being an intervention recommendation model. In even further embodiments, the diagnostic/intervention recommendation model comprises more than two distinct models. For example, in some embodiments, the diagnostic/intervention recommendation model can comprise multiple distinct intervention recommendation models, each associated with a specific condition and configured to determine an intervention recommendation for a subject diagnosed with the condition. As discussed in further detail below, the configuration of the diagnostic/intervention recommendation model depends upon how the diagnostic/intervention recommendation model is trained. Therefore, the configuration of the diagnostic/intervention recommendation system 101 also depends upon how the diagnostic/intervention recommendation model is trained.

As shown in FIG. 1, the diagnostic/intervention recommendation system 101 receives inputs of at least one of electronic health record (EHR) data 102 and biomarker data 103. These inputs are processed by the diagnostic/intervention recommendation system 101 to generate and output a diagnosis/intervention recommendation 104. The output of the diagnostic/intervention recommendation system 101 depends on the configuration of the diagnostic/intervention recommendation system 101. For instance, a diagnostic/intervention recommendation system that is configured to both recommend medical diagnoses for subjects, as well as recommend medical interventions for subjects, is configured to output both diagnosis recommendations and intervention recommendations. As another example, a diagnostic/intervention recommendation system that is solely configured to recommend medical diagnoses for subjects is solely configured to output diagnosis recommendations.

The diagnostic/intervention recommendation system 101 can be configured to recommend a diagnosis and/or an intervention for a subject for any medical condition. As discussed above, the configuration of the diagnostic/intervention recommendation system 101, and thus the medical condition for which the diagnostic/intervention recommendation system 101 recommends diagnoses and/or interventions, depends upon how the diagnostic/intervention recommendation model that comprises the diagnostic/intervention recommendation system 101 is trained. More specifically, a diagnostic/intervention recommendation system 101 is configured to determine a diagnosis/intervention recommendation 104 for a particular condition by training the diagnostic/intervention recommendation model that comprises the diagnostic/intervention recommendation system 101 using training data samples associated with the particular condition. In a preferred embodiment, the diagnostic/intervention recommendation system 101 is configured to determine diagnosis/intervention recommendations for acute medical conditions including sepsis, septic shock, refractory septic shock, acute lung injury, acute respiratory distress syndrome, acute renal failure, acute kidney injury, trauma, burns, COVID19, pneumonia, viral infection, and post-operative conditions (e.g., conditions following open heart surgery). However, the diagnostic/intervention recommendation system 101 can be configured to determine diagnosis/intervention recommendations for any condition.

### II.A. Diagnostic/Intervention Recommendation System Inputs

Turning to the inputs of the diagnostic/intervention recommendation system 101, as shown in the embodiment of the system environment 100 in FIG. 1, the diagnostic/intervention recommendation system 101 receives inputs of at least one of EHR data 102 and biomarker data 103. Each of these inputs is discussed in turn below within Sections II.A.1. and II.A.2.

While the description of FIG. 1 below and the remainder of this disclosure discuss inputs to the diagnostic/intervention recommendation system of both EHR data and biomarker data, in some embodiments, EHR data and biomarker data are not both input into the system to determine diagnosis/intervention recommendations. Specifically, in some embodiments, only one of EHR data and biomarker data are input into the diagnostic/intervention recommendation system to train and/or use the system to determine diagnosis/intervention recommendations. For example, one or more of EHR data and biomarker data may be used by the diagnostic/intervention recommendation system based on data availability and/or clinical application. As a specific example, the diagnostic/intervention recommendation system may use only EHR data to provide rapid subject diagnoses recommendations, but may use biomarker data to provide subject intervention recommendations with less time constraints. In alternative embodiments, after the diagnostic/intervention recommendation model comprising the diagnostic/intervention recommendation system has been sufficiently trained using a robust quantity and quality of training data comprising both EHR data and biomarker data, the diagnostic/intervention recommendation system may not require one of EHR data and biomarker data as an input to accurately determine a diagnosis/intervention recommendation for a subject. Rather, in some embodiments in which the diagnostic/intervention recommendation model comprising the diagnostic/intervention recommendation system has been sufficiently trained using both EHR data and biomarker data, the diagnostic/intervention recommendation system can be configured to accurately output a diagnosis/intervention recommendation for a subject based solely on one of the subject's EHR data and biomarker data. In further embodiments, both EHR data and biomarker data are consecutively input into the same diagnostic/intervention recommendation system in any order. In alternative embodiments, EHR data and biomarker data are consecutively input into distinct diagnostic/intervention recommendation systems. For example, EHR data may be input into a first diagnostic/intervention recommendation system as a mechanism for screening subjects, and based on the results of the screening mechanism, biomarker data for select subjects can be input into a second diagnostic/intervention recommendation system. While these alternative embodiments of inputting EHR data and biomarker data into the diagnostic/intervention recommendation system are not referenced throughout the remainder of this disclosure for simplicity and clarity purposes, all information throughout this disclosure is similarly applicable to embodiments in which EHR data and biomarker data are alternatively input into the diagnostic/intervention recommendation system.

### II.A.1. Electronic Health Record (EHR) Data

The electronic health record (EHR) data 102 input into the diagnostic/intervention recommendation system 101 comprises an electronically-recorded set of medical and/or health information for a subject. The EHR data 102 can be shared between a plurality of computing systems. For example, the EHR data 102 can be transmitted between a plurality of computing systems via a network. Transmission of data over a network can include transmission of data via the internet, wireless transmission of data, non-wireless transmission of data (e.g., transmission of data via ethernet), and any other form of data transmission.

The EHR data 102 can comprise any type of medical and/or health data for a subject, and can be collected by any means. For example, in some embodiments, the EHR data 102 can comprise vital signs (e.g., heart rate and blood pressure), radiology images (e.g., CT scans), genomic data, epigenomic data, transcriptomic data, proteomic data, metabolic data, lipidomic data, and any other type of medical and/or health data. Similarly, the EHR data 102 can be collected using clinical laboratory equipment, a consumer medical device, an in vitro diagnostic device (IVD), a therapeutic device (e.g., an infusion pump), a monitoring device such as a wearable device, vital sign monitors, a radiology device, a research-use-only device, and any other means of medical and/or health data collection.

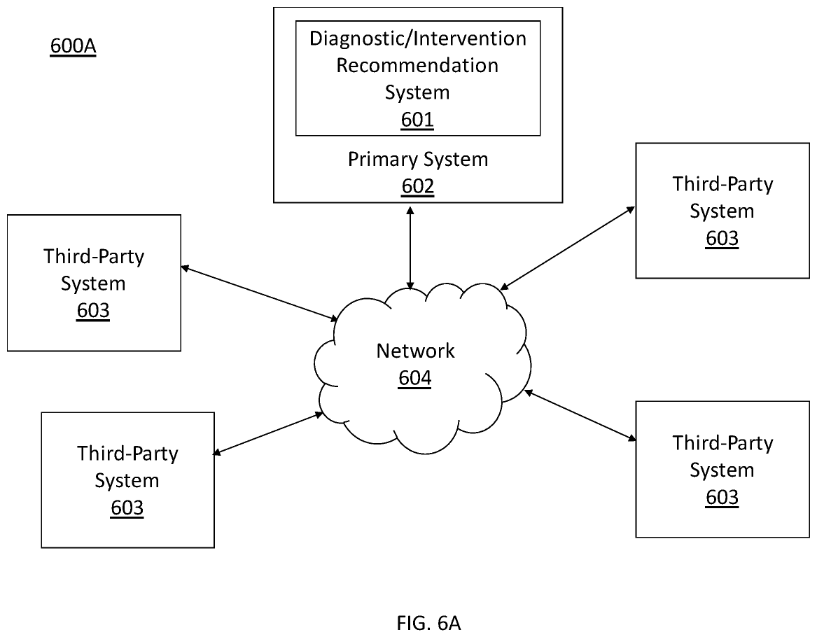

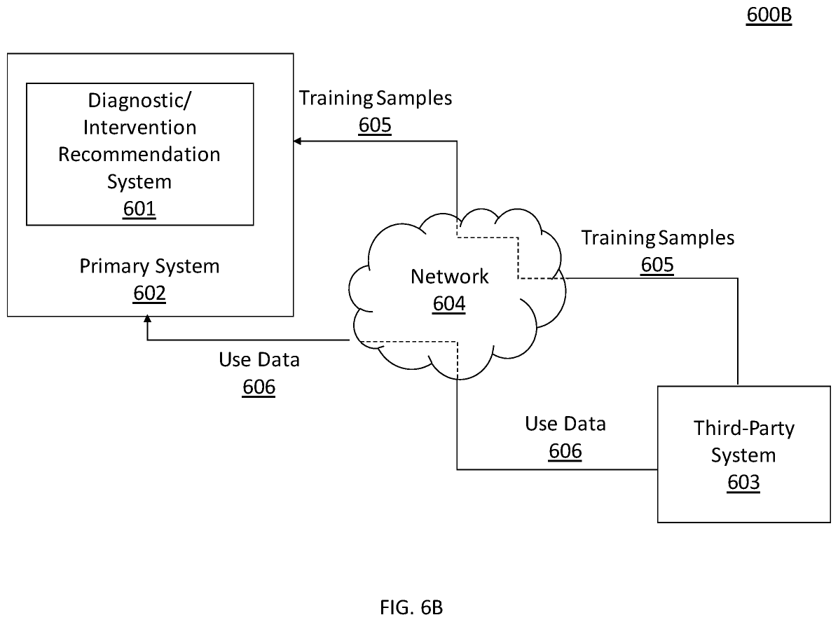

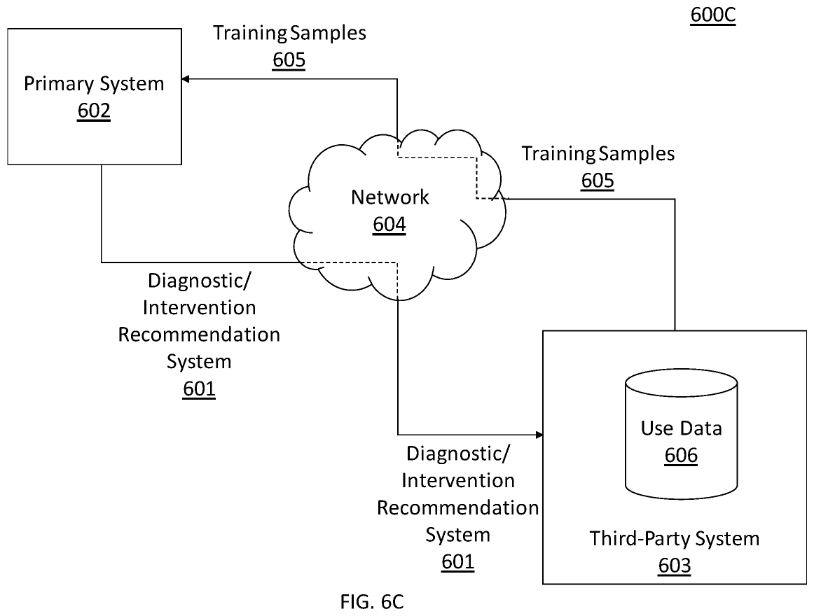

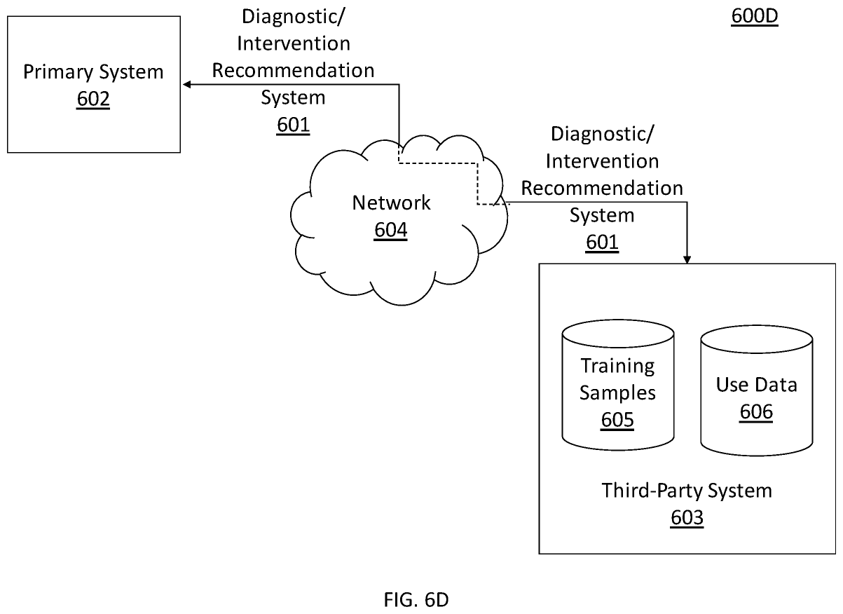

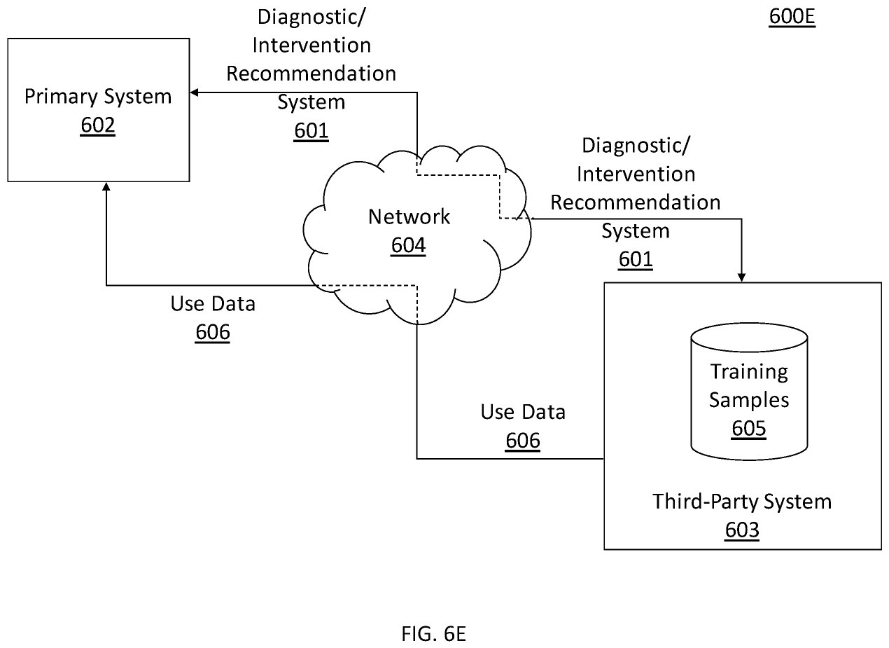

As discussed with regard to FIGS. 6A-E below, the EHR data 102 can be collected and electronically recorded at any site prior to being input into the diagnostic/intervention recommendation system 101. For example, the EHR data 102 can be collected and electronically recorded at a patient care center (e.g., a physician's office, the emergency department of a hospital, the intensive care unit of a hospital, the ward of a hospital), a clinical laboratory, a research laboratory, a consumer medical device, a therapeutic device (e.g., an infusion pump), a monitoring device such as a wearable device (e.g., a heart rate monitor), and any other site. In some embodiments, the EHR data 102 can be electronically recorded at a primary system that also permanently or temporarily stores the diagnostic/intervention recommendation system 101. In such embodiments, the EHR data 102 can be directly input into the diagnostic/intervention recommendation system 101 at the primary system. In alternative embodiments, the EHR data 102 can be electronically recorded at a third-party system remote from the primary system that permanently or temporarily stores the diagnostic/intervention recommendation system 101. In such embodiments, the EHR data 102 can be transmitted via a network from the remote third-party system to the primary system to be received as an input to the diagnostic/intervention recommendation system 101.

The EHR data 102 can also be obtained by the diagnostic/intervention recommendation system 101 from any private, public, and/or commercial source of EHR data. For example, the EHR data 102 can be obtained from a private medical and/or health record and/or middleware system including a patient care center record system, a clinical laboratory record system, a research laboratory record system, such as EPIC®, Cerner®, Allscripts®, MedMined™, Beaker®, and Data Innovations®, and any alternative private medical and/or health record and/or middleware system. The EHR data 102 can also be obtained from any publicly- and/or commercially-available source of EHR data, including published medical record databases and scientific publications such as PhysioNet datasets including the Multiparameter Intelligent Monitoring in Intensive Care (MIMIC) datasets, Philips eICU datasets, and National Heart, Lung, and Blood Institute Biospecimen and Data Repository Information Coordinating Center (BioLINCC) datasets.

In certain embodiments, the EHR data 102 received by the diagnostic/intervention recommendation system 101 comprises an entire EHR dataset for a subject. However, in alternative embodiments, the EHR data 102 received by the diagnostic/intervention recommendation system 101 comprises a select subset of the EHR data stored for a subject. For example, in some embodiments, the EHR data 102 received by the diagnostic/intervention recommendation system 101 comprises a select type of EHR data for a subject. For instance, the EHR data 102 may solely comprise respiratory rate(s) for a subject. Similarly, in some embodiments, the EHR data 102 received by the diagnostic/intervention recommendation system 101 can comprise EHR data received for a subject during a specified period of time. For example, in some embodiments, the EHR data 102 may solely comprise data received for a subject over a 24-hour period of time.

In certain embodiments discussed in further detail below with regard to FIGS. 6A-E, the EHR data 102 can be received from multiple, distinct third-party sources. In such embodiments, the EHR data 102 can be represented in multiple, distinct data formats. For instance, EHR data for different subjects can be organized within different structures. As an example, in some embodiments, EHR data can be organized in delimited flat files, structured documents (e.g., JSON formatted documents), or relational databases. Furthermore, the labeling of EHR data within these different structures can differ as well. For example, in a first structure, heart rate data may be labeled as “HR,” while in a second, different structure, heart rate data may be labeled as “heart rate,” while in yet a third, different structure, heart rate data may be labeled in code. Even further, EHR data can be stored in different units. For example, a first set of EHR data describing temperature may be recorded in Fahrenheit units, while a second set of EHR data describing temperature may be recorded in Celsius units. To render all of these distinct data formats compatible with one another such that the data can be merged to form a single dataset and can be input into the diagnostic/intervention recommendation model, the distinct data formats can be transformed into a common data format. In some embodiments, the distinct data formats can be transformed into a common data format using a publicly-available data transformation model such as, for example, the OMOP Common Data Model.

In certain embodiments, prior to inputting the EHR data 102 into the diagnostic/intervention recommendation model that comprises the diagnostic/intervention recommendation system 101, the EHR data 102 can be combined to create new EHR data 102. For example, the EHR data 102 can be used to create new EHR data 102 describing data trends over time. As another example, the EHR data 102 can be used to create new EHR data 102 comprising ratios or differences between different EHR data variables. In such embodiments, this new, combined EHR data 102 can be input into the model.

In further embodiments, prior to inputting the EHR data 102 into the diagnostic/intervention recommendation model that comprises the diagnostic/intervention recommendation system 101, the EHR data 102 is encoded. Specifically, in some embodiments, the EHR data 102 is encoded prior to being input into the diagnostic/intervention recommendation system 101. In alternative embodiments, the diagnostic/intervention recommendation system 101 contains an encoding module, and the EHR data 102 is encoded by the encoding module following input into the diagnostic/intervention recommendation system 101, but prior to input into the diagnostic/intervention recommendation model of the diagnostic/intervention recommendation system 101. As one example, EHR data describing a heart rate of 60 beats/minute can be encoded in an array of bits as [111100]. As another example, EHR data 102 can be encoded via K-means clustering. K-means clustering can serve to both de-identify subject EHR data, as well as to prevent effects of data-drift. For example, in a case in which EHR data 102 describing mean and median subject body weight steadily increases, the EHR data can continuously undergo K-means clustering, and each identified cluster can be assigned a numeric index. Then, the actual subject body weight values are associated with the numeric indices, and can fluctuate over time and geography. Alternative methods of encoding the EHR data 102 prior to input into the diagnostic/intervention recommendation model of the diagnostic/intervention recommendation system 101 may also be used.

### II.A.2. Biomarker Data

The biomarker data 103 input into the diagnostic/intervention recommendation system 101 comprises data describing the presence or absence of one or more measurable substances in a sample from a subject.

The biomarker data 103 can comprise any measurable substance from any sample from a subject, and can be determined by any means. In a preferred embodiment, the sample from the subject that is used to determine the biomarker data 103 comprises at least one of a blood sample, a urine, stool, bronchial lavage, tissue, mucus, or other bodily sample. In some embodiments, the sample from the subject that is used to determine the biomarker data 103 is collected by one or more of a FDA-cleared, commercially-available sample collection, transport, and processing device. For example, the sample from the subject can be collected using a Vacutainer® tube or a PAXgene® tube. However, in alternative embodiments, the sample from the subject can comprise any alternative sample, and can be collected by any other means.

Similarly, in a preferred embodiment, the biomarker data 103 can comprise at least one of genomic data, epigenomic data, transcriptomic data, proteomic data, metabolic data, and lipidomic data. In further embodiments, the biomarker data 103 can comprise a quantification of expression of each of a plurality of genes in a specified gene panel. In such embodiments, the biomarker data 103 can comprise a quantification of at least one of expression of RNA transcribed from each of the plurality of genes in the specified gene panel, and expression of proteins translated from each of the plurality of genes in the specified gene panel. In alternative embodiments, the biomarker data 103 can comprise data describing the presence or absence of any other measurable substance in a sample from a subject.

The biomarker data 103 can be determined from the subject's sample using clinical laboratory equipment, an in vitro diagnostic device (IVD), a research-use-only device, and any other means of biomarker data determination or collection. In embodiments in which the biomarker data 103 comprises proteomic data, the biomarker data 103 can be determined from the subject's sample by at least one of mass spectrometry and immunoassay. In embodiments in which the biomarker data 103 comprises proteomic data, the biomarker data 103 can be determined from the subject's sample by at least one of mass spectrometry and immunoassay. In embodiments in which the biomarker data 103 comprises genomic data, the biomarker data 103 can be determined from the subject's sample by exome and/or whole genome nucleotide sequencing. In embodiments in which the biomarker data 103 comprises transcriptomic data, the biomarker data 103 can be determined from the subject's sample by microarray, RNA sequencing, and/or RT-qPCR.

As discussed with regard to FIGS. 6A-E below, similar to EHR data 102, the sample from the subject used to determine the biomarker data 103 can be collected at any site, and the biomarker data 103 can be determined using the collected sample at any site, prior to being input into the diagnostic/intervention recommendation system 101. For example, the sample from the subject can be collected at a patient care center (e.g., a physician's office, a hospital), a clinical laboratory, a CLIA-certified laboratory, a research laboratory, a remote location, and any other site. Similarly, the biomarker data 103 can be determined using the collected sample at a patient care center (e.g., a physician's office, a hospital), a clinical laboratory, a CLIA-certified laboratory, a research laboratory, a remote location, and any other site. In certain embodiments, the biomarker data 103 for a subject is determined at the same site at which the sample from the subject was collected. For example, the biomarker data 103 can be obtained from a sample from the subject on-site at a patient care center at which the subject provided the sample. In alternative embodiments, the biomarker data 103 for a subject can be determined at a different site from which the sample from the subject was collected. For example, the biomarker data 03 can be obtained from a sample from the subject off-site from a patient care center at which the subject provided the sample.

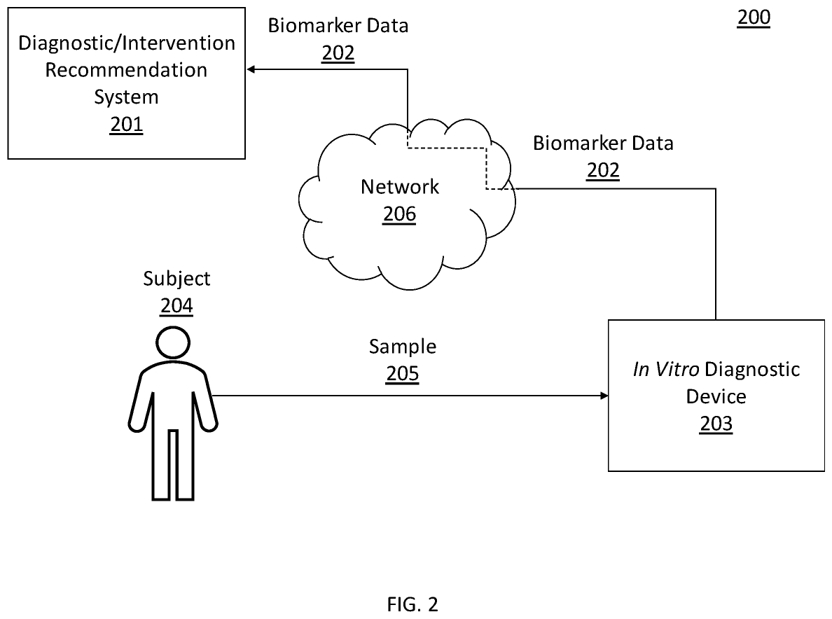

Also similar to the EHR data 102, in certain embodiments, the biomarker data 103 can be determined using a subject's sample at a primary system that also permanently or temporarily stores the diagnostic/intervention recommendation system 101. In such embodiments, the biomarker data 103 can be directly input into the diagnostic/intervention recommendation system 101 at the primary system. In alternative embodiments, the biomarker data 103 can be determined at a third-party system remote from the primary system that permanently or temporarily stores the diagnostic/intervention recommendation system 101. In such embodiments, the biomarker data 103 can be transmitted via a network from the remote third-party system to the primary system to be received as an input to the diagnostic/intervention recommendation system 101. Transmission of data over a network can include transmission of data via the internet, wireless transmission of data, non-wireless transmission of data (e.g., transmission of data via ethernet), and any other form of data transmission. An example in which network transmission of biomarker data occurs is provided and discussed below with regard to FIG. 2.

The biomarker data 103 can also be obtained by the diagnostic/intervention recommendation system 101 from any private, public, and/or commercial source. For example, the biomarker data 103 can be obtained from a private medical record system including a patient care center record system, a clinical laboratory record system, a research laboratory record system, a hospital record system, a research institute record system, and/or a private company record system. The biomarker data 103 can also be obtained from any publicly- and/or commercially-available source, including published medical records, biorepository databases, and/or scientific publications such as The National Center for Biotechnology Information Gene Expression Omnibus (GEO) Database Repository Of High Throughput Gene Expression Data, The European Molecular Biology Laboratory-European Bioinformatics Institute (EMBL-EBI) ArrayExpress Archive of Functional Genomics Data, The Inflammation And The Host Response To Injury Glue Grant Datasets, The National Heart, Lung, And Blood Institute Biospecimen And Data Repository Information Coordinating Center (BioLINCC) Datasets, and any other data system or repository.

In certain embodiments discussed in further detail below with regard to FIGS. 6A-E, the biomarker data 103 can be received from multiple, distinct third-party sources. In such embodiments, the biomarker data 103 can be represented in multiple, distinct data formats. For instance, biomarker data for different subjects can be organized within different structures. As an example, in some embodiments, biomarker data can be organized in delimited flat files, structured documents (e.g., JSON formatted documents), or relational databases. Furthermore, the labeling of biomarker data within these different structures can differ as well. For example, in a first structure, genetic expression data for a gene A may be labeled as “gene A,” while in a second, different structure, genetic expression data for the gene A may be labeled simply as “A.” Even further, biomarker data can be stored in different units. To render all of these distinct data formats compatible with one another such that the data can be merged to form a single dataset and can be input into the diagnostic/intervention recommendation model, the distinct data formats can be transformed into a common data format. In some embodiments, the distinct data formats can be transformed into a common data format using a publicly-available data transformation model such as, for example, the OMOP Common Data Model.

In some embodiments, prior to inputting the biomarker data 103 into the diagnostic/intervention recommendation model that comprises the diagnostic/intervention recommendation system 101, the biomarker data 103 can be modified to create new biomarker data 103. For example, the biomarker data 103 can be normalized to follow a particular distribution prior to being input into the model.

In further embodiments, prior to inputting the biomarker data 103 into the diagnostic/intervention recommendation model that comprises the diagnostic/intervention recommendation system 101, the biomarker data 103 is encoded. Specifically, in some embodiments, the biomarker data 103 is encoded prior to being input into the diagnostic/intervention recommendation system 101. In alternative embodiments, the diagnostic/intervention recommendation system 101 contains an encoding module, and the biomarker data 103 is encoded by the encoding module following input into the diagnostic/intervention recommendation system 101, but prior to input into the diagnostic/intervention recommendation model of the diagnostic/intervention recommendation system 101. As one example, biomarker data describing the presence of a particular protein in a blood sample from a subject can be encoded by a bit of ‘1’. As another example, biomarker data 103 can be encoded via K-means clustering as described above in Section II.A.1. Alternative methods of encoding the biomarker data 103 prior to input into the diagnostic/intervention recommendation model of the diagnostic/intervention recommendation system 101 may also be used.

In certain embodiments in which a subject's biomarker data 103 is determined from the subject's sample using an in vitro diagnostic device (IVD), the biomarker data 103 can be automatically received by the diagnostic/intervention recommendation system 101 directly from the IVD. Such an embodiment is illustrated in FIG. 2. FIG. 2 is a block diagram of a system environment 200 for a diagnostic/intervention recommendation system 201 configured to determine at least one of a medical diagnosis recommendation for a subject and a medical intervention recommendation for a subject using biomarker data 202 automatically received from directly an in vitro diagnostic device 203, in accordance with an embodiment.

As shown in FIG. 2, a subject 204 provides a sample 205 to the IVD 203, which processes the sample 205 to determine the biomarker data 202 for the subject 204. This biomarker data is then automatically provided directly to the diagnostic/intervention recommendation system 201, where it is used to determine at least one of a diagnosis recommendation and an intervention recommendation for the subject 204. In certain embodiments, the biomarker data 202 can be provided directly to the diagnostic/intervention recommendation system 201 as un-processed data.

In the embodiment of the environment 200 shown in FIG. 2, the biomarker data 202 is transmitted from the IVD 203 to the diagnostic/intervention recommendation system 201 via a network 206. However, in alternative embodiments, the biomarker data 202 can be provided to the diagnostic/intervention recommendation system 201 via any alternative means. For example, in some alternative embodiments, the IVD 203 can be connected to a primary system that stores the diagnostic/intervention recommendation system 201 via a wire connection, such that the IVD 203 is able to provide the biomarker data 202 directly to the diagnostic/intervention recommendation system 201 via the wire connection.

### II.B. Diagnostic/Intervention Recommendation System Outputs

Returning to FIG. 1, and turning to the output of the diagnostic/intervention recommendation system 101, the diagnostic/intervention recommendation system 101 outputs the diagnosis/intervention recommendation 104 determined by the diagnostic/intervention recommendation system 101 based on the inputs.

A diagnosis recommendation output by the diagnostic/intervention recommendation system 101 comprises a recommendation of a medical condition of a subject. In some embodiments, the diagnosis recommendation output by the diagnostic/intervention recommendation system 101 can also include a likelihood that the recommended diagnosis is accurate.

An intervention recommendation output by the diagnostic/intervention recommendation system 101 comprises a recommendation of a medical intervention for a subject. The intervention recommendation can be a pharmaceutical and/or a non-pharmaceutical intervention recommendation. In embodiments in which an intervention recommendation output by the diagnostic/intervention recommendation system 101 is a pharmaceutical intervention recommendation, the intervention recommendation can comprise at least one of a selection, dosage, timing, starting instructions, monitoring, and stopping instructions of one or more pharmaceutical compounds, drugs, and biologics. An example of a non-pharmaceutical intervention recommendation that can be output by the diagnostic/intervention recommendation system 101 is a ventilator pressure adjustment. As another example, a non-pharmaceutical intervention recommendation may be the collection of a biospecimen from the subject and/or the collection of electronic health record data from the subject. In some embodiments, an intervention recommendation can comprise a recommendation to not perform a particular intervention. For example, an intervention recommendation can comprise a recommendation to not administer corticosteroids. In additional embodiments, providing an intervention recommendation can comprise providing no intervention recommendation, for example, if the diagnostic/intervention system 101 lacks sufficient data to provide an intervention recommendation, or if no intervention is recommended for the subject.

As briefly discussed above, the diagnostic/intervention recommendation system 101 can output a diagnosis recommendation for any medical condition and/or a medical intervention recommendation for a subject with any medical condition. However, the specific output of the diagnostic/intervention recommendation system 101 depends upon the configuration of the diagnostic/intervention recommendation system 101, which in turn depends upon the set of training data used to train the diagnostic/intervention recommendation system 101. For example, a diagnostic/intervention recommendation system trained to diagnose medical conditions in subjects outputs medical diagnosis recommendations, but not intervention recommendations.

The diagnosis/intervention recommendation 104 is determined by the diagnostic/intervention recommendation system 101, and thus is output by the diagnostic/intervention recommendation system 101 at the site at which the diagnostic/intervention recommendation system 101 determines the diagnosis/intervention recommendation 104. As discussed below in detail with regard to FIGS. 6A-E, the diagnostic/intervention recommendation system 101 can determine and output the diagnosis/intervention recommendation 104 at a system at which one or more of the inputs are stored and/or are input into the diagnostic/intervention recommendation system 101. Alternatively, the diagnostic/intervention recommendation system 101 can determine and output the diagnosis/intervention recommendation 104 at a system that is remote from a system at which one or more of the inputs are stored and/or are input into the diagnostic/intervention recommendation system 101.

The diagnosis/intervention recommendation 104 output by the diagnostic/intervention recommendation system 101 can be provided in any form. In some embodiments, the diagnosis/intervention recommendation 104 is displayed (e.g., digitally displayed). In further embodiments, the diagnosis/intervention recommendation 104 can be automatically electronically stored, automatically transmitted via a network to a remote system, and/or returned by any other method. Transmission of the diagnosis/intervention recommendation 104 over a network can include transmission of diagnosis/intervention recommendation 104 via the internet, via wireless transmission, via non-wireless transmission (e.g., via ethernet), or any other form of transmission. In embodiments in which the diagnosis/intervention recommendation 104 at least in part comprises an intervention recommendation, the diagnosis/intervention recommendation 104 can comprise instructions for performing the recommended intervention. In further embodiments in which the diagnosis/intervention recommendation 104 at least in part comprises an intervention recommendation, the diagnosis/intervention recommendation 104 can comprise automatic performance of the recommended intervention.

As mentioned above, in a preferred embodiment, the diagnostic/intervention recommendation system 101 is configured to determine diagnosis/intervention recommendations 104 for acute medical conditions including sepsis, septic shock, refractory septic shock, acute lung injury, acute respiratory distress syndrome (ARDS), acute renal failure, acute kidney injury (AKI), trauma, burns, COVID19, pneumonia, viral infection, and post-operative conditions including open heart surgery. In some particular embodiments in which the diagnostic/intervention recommendation system 101 is configured to determine intervention recommendations for subjects diagnosed with sepsis, the intervention recommendations output by the diagnostic/intervention recommendation system 101 can include administration of vasopressors (e.g., vasopressin and norepinephrine), fluids (e.g., aggressive, restrictive, crystalloid solutions, balance solutions), antibiotic therapy, corticosteroids (e.g., hydrocortisone), vitamins such as thiamine and ascorbic acid (e.g., vitamin B1 and vitamin C), immunoglobulins, immunostimulatory therapies (e.g., granulocyte-macrophage colony stimulating factor, interferon-gamma, interleukin-7, anti-programmed cell death protein 1, Thymosin alpha I, GM-CSF), thrombomodulin, Xigris®, adjusted ventilator settings, adjusted renal replacement therapy settings, adjusted extracorporeal removal (e.g., hemofiltration) settings, and/or any other pharmaceutical or non-pharmaceutical intervention. Similarly, in embodiments in which the diagnostic/intervention recommendation system 101 is configured to determine intervention recommendations for subjects diagnosed with ARDS, the intervention recommendations output by the diagnostic/intervention recommendation system 101 can include administration of fluids (e.g., aggressive, restrictive, crystalloid solutions, balance solutions), neuromuscular blockade, inhaled nitric oxide, corticosteroids (e.g., hydrocortisone), statins (e.g., simvastatin), surfactant replacement, neutrophil elastase inhibition therapies, anticoagulation therapies, nonsteroidal anti-inflammatory agents (e.g., ketoconazole and lisofylline), albuterol, antioxidants (e.g., procysteine [1-2-oxothiazolidine-4-carboxylic acid]), adjusted ventilator settings, adjusted renal replacement therapy settings, adjusted extracorporeal membrane oxygenation (ECMO) settings, and/or any other pharmaceutical or non-pharmaceutical intervention. In embodiments in which the diagnostic/intervention recommendation system 101 is configured to recommend diagnoses for subjects diagnosed with AKI, the diagnosis recommendations output by the diagnostic/intervention recommendation system 101 can include etiology identification (e.g., pre-renal or renal AKI). In embodiments in which the diagnostic/intervention recommendation system 101 is configured to determine intervention recommendations for subjects diagnosed with AKI, the intervention recommendations output by the diagnostic/intervention recommendation system 101 can include administration of vasopressors (e.g., vasopressin, norepinephrine, and angiotensin II), alkaline phosphatase, thiamine, statins (e.g., simvastatin), N-acetyl-cysteine, erythropoietin, steroids, reltecimod, L-carnitine, an adsorptive filter, fluids (e.g., aggressive, restrictive, crystalloid solutions, balance solutions), adjusted ventilator settings, adjusted renal replacement therapy settings, adjusted extracorporeal membrane oxygenation (ECMO) settings, and/or any other pharmaceutical or non-pharmaceutical intervention.

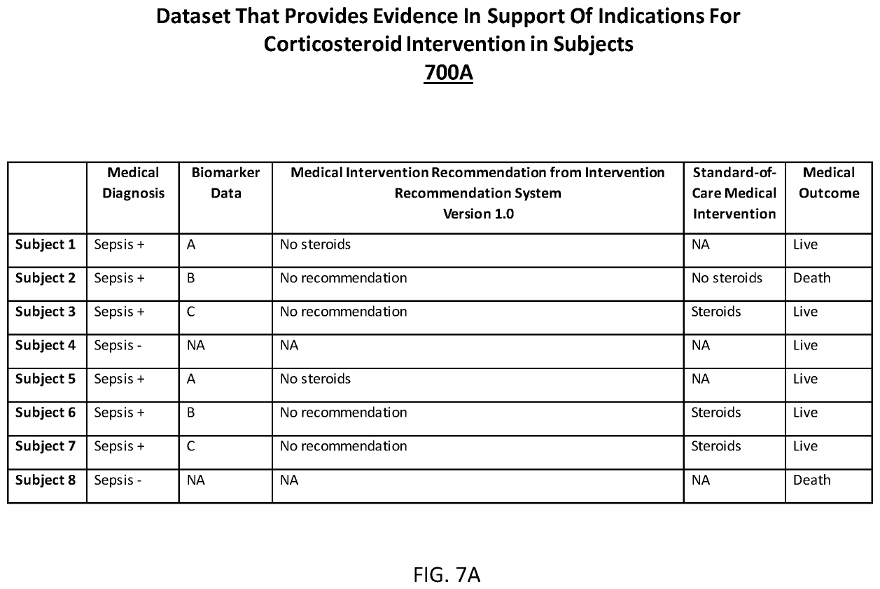

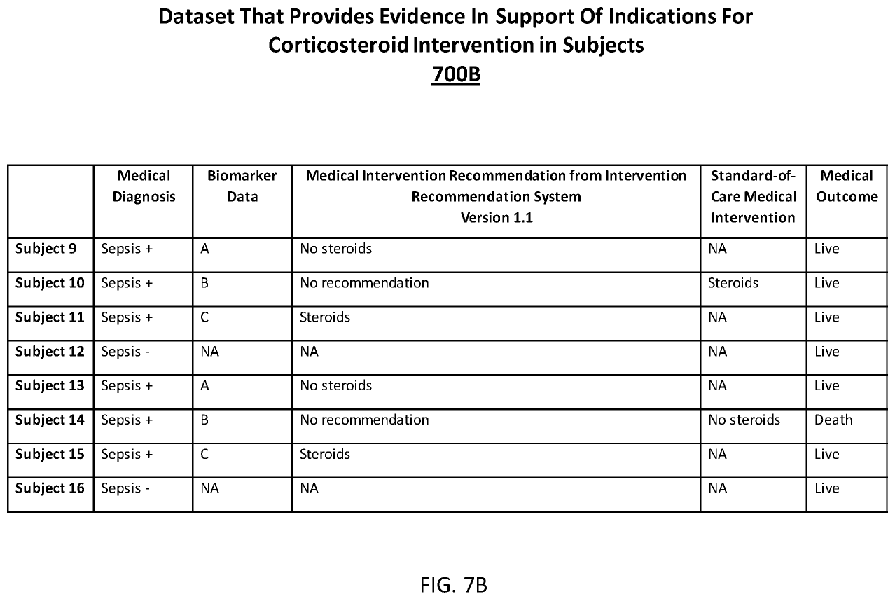

In further embodiments discussed in detail below with regard to FIGS. 7A-B, in addition to outputting the diagnosis/intervention recommendation 104, the diagnostic/intervention recommendation system 101 can also be configured to generate a dataset that provides evidence in support of an indication for a particular medical intervention for a medical condition. This dataset can be used for example, in expediting regulatory approval of the intervention for the condition and the indication. Such embodiments are discussed in further detail below with regard to FIGS. 7A-B.

### II.C. Diagnostic/Intervention Recommendation System Architecture

Turning next to FIG. 3, FIG. 3 is a block diagram of an architecture of a diagnostic/intervention recommendation system 300 configured to determine at least one of a medical diagnosis recommendation for a subject, a cohort/randomization designation for a subject, and a medical intervention recommendation for a subject, in accordance with an embodiment. The diagnostic/intervention recommendation system 300 includes a training module 301, a data store 302, a data management module 303, and a diagnostic/intervention recommendation model 304. The diagnostic/intervention recommendation model 304 further comprises a function 305 and a set of parameters 306. In other embodiments, the diagnostic/intervention recommendation system 300 may include additional, fewer, or different components for various applications. Similarly, the functions can be distributed among the modules in a different manner than is described here. Conventional components such as network interfaces, security functions, load balancers, failover servers, management and network operations consoles, and the like are not shown so as to not obscure the details of the system architecture.

### II.C.1. Training Module

The training module 301 constructs the diagnostic/intervention recommendation model 304 based on a training dataset. In general, the diagnostic/intervention recommendation model 304 comprises a function 305 and a plurality of parameters 306. The function 305 captures the relationship between independent variables (e.g., EHR and biomarker data) and dependent variables (e.g., diagnosis/intervention recommendation) in the training dataset. The parameters 306 modify the function 305, and are identified during training of the diagnostic/intervention recommendation model 304 based on the training dataset.

Construction (e.g., identification of the parameters 306) of the diagnostic/intervention recommendation model 304 using the training dataset is based on the type of the diagnostic/intervention recommendation model 304. As discussed in further detail below in Section II.C.4., the diagnostic/intervention recommendation model 304 can be any model for which the parameters 306 comprising the model are learned by a computer based on the training dataset. The parameters 306 are learned by a computer because it would be too difficult or too inefficient for the parameters 306 to be identified by a human based on the training dataset due to the size and/or complexity of the training dataset. In some embodiments, the diagnostic/intervention recommendation model 304 can be a discretely programmed model (e.g., a generalized linear model, a gradient boosting classifier, a neural network, a support vector machine, or a discriminative factor model). In alternative embodiments, the diagnostic/intervention recommendation model 304 can be learned via unsupervised learning (e.g., latent class analysis, K-means clustering, principal component analysis, or unsupervised neural network). In further embodiments, the diagnostic/intervention recommendation model 304 can be learned via supervised learning. For example, the diagnostic/intervention recommendation model 304 can be a classifier, a regression model, or a supervised neural network.

The training dataset used to construct the diagnostic/intervention recommendation model 304 depends on the type of the diagnostic/intervention recommendation model 304. As discussed in further detail below in Section II.C.2., in general, for each model type, the training dataset comprises a plurality of training samples. Each training sample i from the training dataset is associated with a retrospective subject, and comprises EHR data and biomarker data for the retrospective subject. A retrospective subject is a subject for whom at least EHR data and/or biomarker data is known.

To construct the diagnostic/intervention recommendation model 304, each training sample i from the training dataset is input into the diagnostic/intervention recommendation model 304. The diagnostic/intervention recommendation model 304 processes these inputs as if the model were being routinely used to generate a diagnosis/intervention recommendation. However, depending on the type of the diagnostic/intervention recommendation model 304, each training sample i of the training dataset may comprise additional components.

### Unsupervised Learning

In embodiments in which the diagnostic/intervention recommendation model 304 is learned via unsupervised learning, the model is trained based on the basic training dataset described above, with no additional components such as retrospective subjects' medical outcomes. For example, in embodiments in which the diagnostic/intervention recommendation model 304 is constructed via K-means clustering, an optimal number and configuration of clusters that both minimize differences between the training samples within each cluster, and maximize differences between the training samples between clusters, are determined. Specifically, in training the diagnostic/intervention recommendation model 304 using K-means clustering, parameters θ that define the centroid of each cluster in the variable space of the diagnostic/intervention recommendation model 304 are learned. Collectively, these parameters θ comprise the parameters 306, and mathematically modify the function 305 to specify the dependence between independent variables (e.g., EHR and biomarker data) and dependent variables (e.g., diagnosis/intervention recommendations). The clinical significance of each cluster can be determined by examining the inputs to the diagnostic/intervention recommendation model 304 that affect assignment of the inputs to clusters.

### Supervised Learning

In contrast, in embodiments in which the diagnostic/intervention recommendation model 304 is learned via supervised learning, each training sample i from the training dataset further includes a retrospective diagnosis/intervention for the retrospective subject associated with the training sample, as well as a known, retrospective medical outcome of the retrospective subject following receipt of the medical diagnosis/intervention. Specifically, in embodiments in which the diagnostic/intervention recommendation model 304 is configured to determine a diagnosis recommendation for a subject, a retrospective medical diagnosis of the retrospective subject, as well as a known, retrospective medical outcome of the retrospective subject following receipt of the medical diagnosis, are included in each training sample i and are also input into the model during training. Similarly, in embodiments in which the diagnostic/intervention recommendation model 304 is configured to determine an intervention recommendation for a subject, a retrospective medical intervention provided to the retrospective subject, as well as a known, retrospective medical outcome of the retrospective subject following receipt of the medical intervention, are included in each training sample i and are also input into the model during training. In other words, in embodiments in which the diagnostic/intervention recommendation model 304 is learned via supervised learning, the model is trained based in part on the known medical outcomes of retrospective subjects associated with the training dataset.

As discussed in further detail below, the retrospective medical outcomes of the retrospective subjects that are input into the model during training can be any one or more medical outcome(s), and can be selected based on the medical outcome of the subject that the diagnostic/intervention recommendation model 304 seeks to optimize. For example, medical outcome can include a number of days that a subject requires use of a ventilator, subject mortality, and/or any other subject medical outcome metric. Additional subject medical outcomes are discussed and defined in detail below with regard to Section VIII.A. As another example, the retrospective medical outcomes that are used to train the model can be weighted combinations of multiple retrospective medical outcomes.

In addition to training the diagnostic/intervention recommendation model 304 to optimize subject medical outcomes, in some embodiments, the diagnostic/intervention recommendation model 304 can be trained to optimize other performance metrics. For instance, in embodiments in which the diagnostic/intervention recommendation model 304 at least in part comprises a diagnostic recommendation model, the diagnostic/intervention recommendation model 304 can also be trained to optimize fundamental predictive diagnostic metrics, such as, for example, sensitivity and specificity of the diagnosis recommendations. Furthermore, the diagnostic/intervention recommendation model 304 can be trained to optimize for any weighted combination of performance metrics, including fundamental predictive diagnostic metrics and/or subject medical outcomes. Section VIII.A. below discusses and defines fundamental predictive diagnostic metrics that can be optimized during model training.

Turning back to training of the diagnostic/intervention recommendation model 304 using retrospective medical outcomes, after each iteration of the diagnostic/intervention recommendation model 304 using a training sample i in the training dataset, the difference between the diagnosis/intervention recommendation output by the model and the retrospective diagnosis/intervention of the retrospective subject is determined in view of the retrospective medical outcome of the retrospective subject. Specifically, in embodiments in which the diagnostic/intervention recommendation model 304 is configured to determine a diagnosis recommendation for a subject, the model determines the difference between the diagnosis recommendation output by the model and the retrospective diagnosis in view of the retrospective medical outcome of the retrospective subject following the retrospective diagnosis. Similarly, in embodiments in which the diagnostic/intervention recommendation model 304 is configured to determine an intervention recommendation for a subject, the model determines the difference between the intervention recommendation output by the model and the retrospective intervention in view of the retrospective medical outcome of the retrospective subject following the retrospective intervention.

Then, the diagnostic/intervention recommendation model 304 seeks to maximize improvement of the retrospective medical outcome by adjusting this difference between the diagnosis/intervention recommendation output by the model and the retrospective diagnosis/intervention. Specifically, in embodiments in which the diagnostic/intervention recommendation model 304 is configured to determine a diagnosis recommendation for a subject, the model seeks to maximize improvement of the retrospective medical outcome by adjusting the difference between the diagnosis recommendation output by the model and the retrospective diagnosis. As mentioned above, in embodiments in which the diagnostic/intervention recommendation model 304 is configured to determine a diagnosis recommendation for a subject, the model can also or alternatively maximize improvement of one or more fundamental predictive diagnostic metrics by adjusting the difference between the diagnosis recommendation output by the model and the retrospective diagnosis. Similarly, in embodiments in which the diagnostic/intervention recommendation model 304 is configured to determine an intervention recommendation for a subject, the model seeks to maximize improvement of the retrospective medical outcome by adjusting the difference between the intervention recommendation output by the model and the retrospective intervention.

To adjust this difference, the diagnostic/intervention recommendation model 304 can minimize or minimize a loss function for the diagnostic/intervention recommendation model 304. The loss function l(uᵢ∈s,yᵢ∈s;θ) represents discrepancies between values of dependent variables uᵢ∈s for one or more training samples i in the training data S (e.g., known, retrospective diagnoses/interventions), and dependent variables yᵢ∈s for the training samples i generated by the diagnosis/intervention recommendation model 304 (e.g., predicted diagnosis/intervention recommendations). In simple terms, the loss function represents the difference between diagnosis/intervention recommendations output by the diagnosis/intervention recommendation model 304 and the known, retrospective diagnoses/intervention in the training dataset. There are a plurality of loss functions known to those skilled in the art, and any one of these loss functions can be utilized in generating the diagnostic/intervention recommendation model 304.

By minimizing or maximizing the loss function with respect to θ, values for a set of parameters θ can be determined. In some embodiments, the diagnostic/intervention recommendation model 304 can be a parametric model in which the set of parameters θ comprise the parameters 306 and mathematically modify the function 305 to specify the dependence between independent variables (e.g., EHR and biomarker data) and dependent variables (e.g., diagnosis/intervention recommendations). In other words, the set of parameters θ determined by minimizing or maximizing the loss function can comprise the set of parameters 306 and can be used to modify the function 305 of the diagnostic/intervention recommendation model 304 such that the medical outcomes of the subjects for which the diagnostic/intervention recommendation model 304 is used to determine diagnosis/intervention recommendations, are optimized. In some embodiments, fundamental predictive diagnostic metrics can also or alternatively be optimized. Typically, the parameters of parametric-type models that minimize or maximize the loss function are determined through gradient-based numerical optimization algorithms, such as batch gradient algorithms, stochastic gradient algorithms, and the like. Alternatively, the diagnostic/intervention recommendation model 304 may be a non-parametric model in which the model structure is determined from the training dataset and is not strictly based on a fixed set of parameters.

### Diagnostic/Intervention Recommendation Model Function and Parameters

In embodiments in which the diagnostic/intervention recommendation model 304 comprises a parametric-model, the model can generally be represented as:

y=ƒ(x^{k};θ) (1A)

where y denotes the diagnosis/intervention recommendation determined by the diagnostic/intervention recommendation model 304, x^{k} denotes the independent variables (e.g., x¹=EHR data and x²=biomarker data), θ denotes the set of parameters 306, and ƒ( ) is the function 305.

In some embodiments, the diagnostic/intervention recommendation model 304 comprises two or more functions. In such embodiments, the model can be represented as:

y=ƒ₁(x₁^{k};*θ₁)*ƒ₂(x₂^{j};θ₂) (1B)

where the indicator “*” represents any mathematical operation (e.g., summation, multiplication, etc.) such that the two functions, ƒ₁ and ƒ₂, are combined to determine y, the diagnosis/intervention recommendation.

In some embodiments, the diagnostic/intervention recommendation model 304 comprises two or more functions where the output of a first function serves as input to a second function. In such embodiments, the model can be represented as:

y=g(ƒ(x^{k};θ)) (1C)

where ƒ is the first function and the output of ƒ serves as input to the second function g.

In some embodiments, the diagnostic/intervention recommendation model 304 comprises a plurality of functions whose outputs serve as input to one or more functions. In such embodiments, the model can be represented as:

y=g(ƒ₁(x₁^{k};θ₁)*ƒ₂(x₂^{j};θ₂)) (1D)

where ƒ₁ and ƒ₂ are the plurality of functions whose output serve as input to an additional function g, which outputs y, the diagnosis/intervention recommendation.

In certain embodiments in which x^{k} denotes multiple different independent variables (e.g., x¹ and x²), the multiple independent variables can be combined prior to being input into the function ƒ( ). For example, independent variables of EHR data and biomarker data can be combined to create a new independent variable prior to being input into the function ƒ( ). For example, a subject's biomarker data in the form of a blood creatine level can be combined with the subject's EHR data in the form of urine output volume to create a new independent variable describing the subject's kidney function. As another example, a subject's EHR data in the form of heart rate can be divided by additional subject EHR data in the form of systolic blood pressure to create a new independent variable describing the subject's shock index. As yet another example, a subject's biomarker data in the form of levels of expression of three different genes may be averaged to create a new independent variable describing the subject's average gene expression level. In alternative embodiments in which x^{k} denotes multiple different independent variables (e.g., x¹ and x²), the different independent variables remain separate and distinct from one another when input into the function ƒ( ).

The function ƒ( ) can be any function, and can comprise any combination of hyperparameters. For example, in some embodiments, the function ƒ( ) can be an affine function given by:

y=ƒ(x^{k};θ)=x^{k}·θ (2)

that linearly combines each independent variable in x^{k} with a corresponding parameter in the set of parameters 306.

As another example, in some embodiments, the function ƒ( ) can be a network function given by:

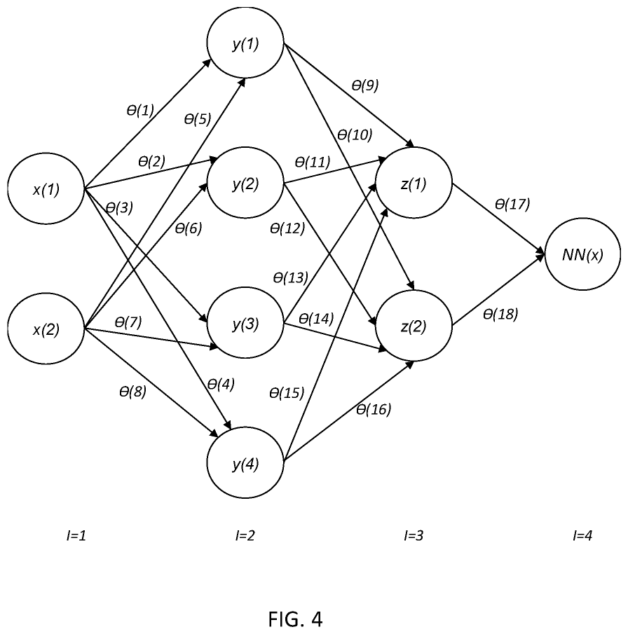

y=ƒ(x^{k};θ)=NN(x^{k};θ) (3)

where NN( ) is a network model. Generally, network models NN( ) can be feed-forward networks, such as artificial neural networks (ANN), convolutional neural networks (CNN), deep neural networks (DNN), and/or recurrent networks, such as long short-term memory networks (LSTM), bi-directional recurrent networks, deep bi-directional recurrent networks, and the like. A network model NN( ) can be defined by any combination of hyperparameters. For example, in a recurrent network, the network can comprise any number of hidden layers, with any number of nodes per layer, and each layer can comprise any layer type, including, but not limited to, a Masking Layer, a Long-Short Term Memory (LSTM) Layer, a Gated Recurrent Units (GRU) Layer, and a Densification Layer. Furthermore, the learning rate of the model can comprise any rate. An example network model NN( ) is discussed in detail below with regard to FIG. 4.

In even further embodiments, the function ƒ( ) can be an ensemble of decision trees, such as a random forest or a gradient boosting classifier. In such embodiments, any number of decision trees may be incorporated into the model, and each decision tree may have any maximum depth. Furthermore, the learning rate of the model can comprise any rate.

In alternative embodiments, the diagnostic/intervention recommendation model 304 comprises distinct functions 305 and distinct sets of parameters 306 for each independent variable x^{k}. For example, in embodiments in which the independent variables include EHR data x¹ and biomarker data x², separate sets of parameters θ¹ and θ² can be determined for each independent variable EHR data x¹ and biomarker data x², respectively. For example, in embodiments in which the diagnostic/intervention recommendation model 304 is learned via supervised learning, as discussed above, the values for a set of parameters θ¹ can be determined by minimizing or maximizing the loss function with respect to θ¹, and the values for a set of parameters θ² can determined by minimizing or maximizing the loss function with respect to θ². The set of parameters θ¹ are then used to modify a first function ƒ(x¹;θ¹), and the set of parameters θ² are used to modify a second function ƒ(x²;θ²). In some embodiments, these distinct functions modified by distinct sets of parameters can remain separate from one another, in effect comprising separate EHR and biomarker models. Alternatively, in some embodiments, these distinct functions modified by distinct sets of parameters can be combined to generate a single diagnosis/intervention recommendation model. In such embodiments, the single diagnostic/intervention recommendation model 304 can be represented as:

y=ƒ(x¹;θ¹)+ƒ(x²;θ²) (4)

where y denotes the diagnosis/intervention recommendation determined by the diagnostic/intervention recommendation model 304, x¹ denotes a first independent variable (e.g., EHR data), x² denotes a second independent variable (e.g., biomarker data), θ¹ denotes a first set of parameters 306, θ² denotes a second set of parameters 306, and ƒ( ) is a function 305.

As discussed above with regard to Equation 1, the function ƒ( ) can be any function. For example, in some embodiments the function ƒ( ) can be an affine function depicted in Equation 2, where x^{k} becomes x¹ or x². Alternatively, the function ƒ( ) can be a network function depicted in Equation 3, where x^{k} becomes x¹ or x². In even further embodiments, the function ƒ( ) can be an ensemble of decision trees, such as a random forest or a gradient boosting classifier. Furthermore, the ƒ( ) functions denoted in Equation 4 are not required to be the same function. For instance, one of the ƒ( ) functions of Equation 4 can be an affine function while the other ƒ( ) function of Equation 4 is a network function.

In some embodiments, during training of the diagnostic/intervention recommendation model 304, one or more training samples i are automatically received by the diagnostic/intervention recommendation system 300 at specified time intervals and the plurality of parameters 306 are automatically identified using the received training samples i at specified time intervals, such that the diagnostic/intervention recommendation model 304 is automatically updated at specified time intervals. In alternative embodiments, during training of the diagnostic/intervention recommendation model 304, one or more training samples i are automatically received by the diagnostic/intervention recommendation system 300 in real-time, near real-time, delayed batch or on demand and the plurality of parameters 306 are automatically identified in-real time using the received training samples i, such that the diagnostic/intervention recommendation model 304 is automatically updated in-real time.

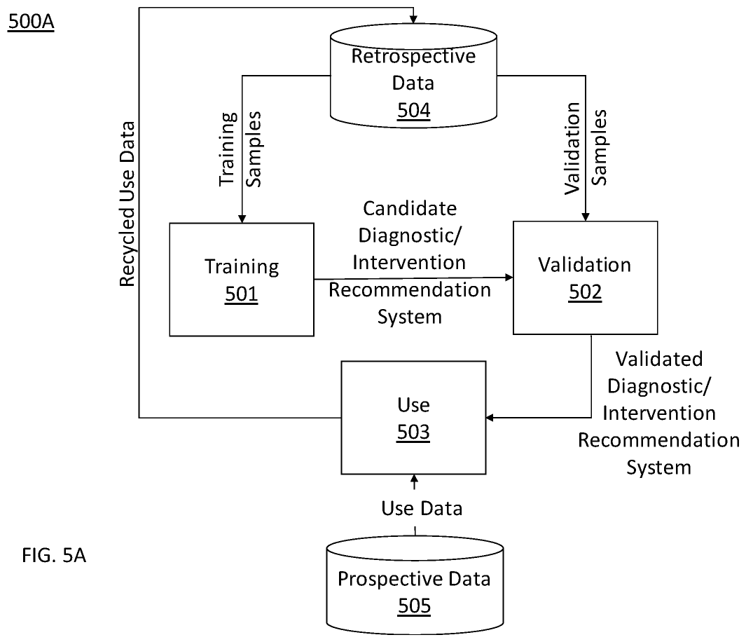

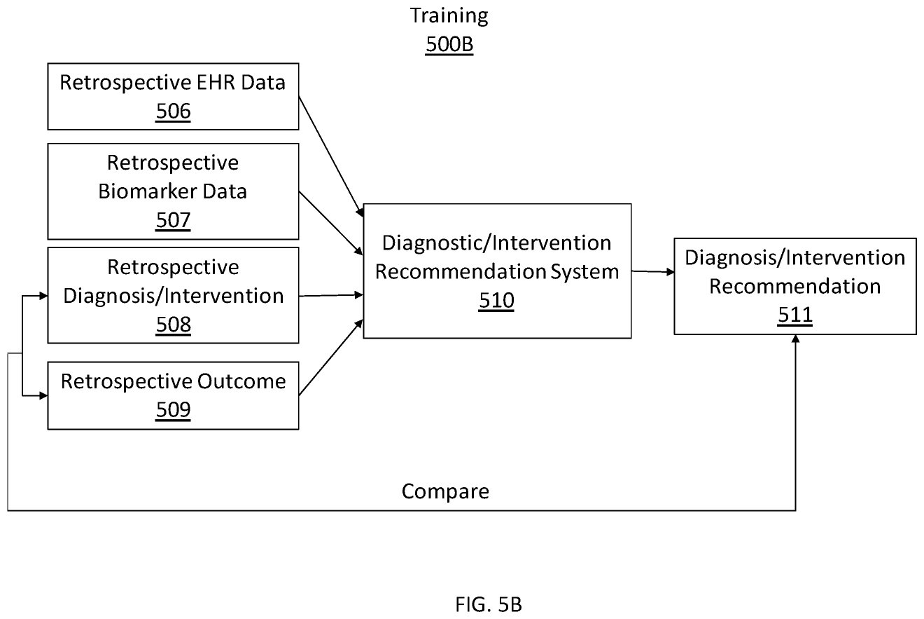

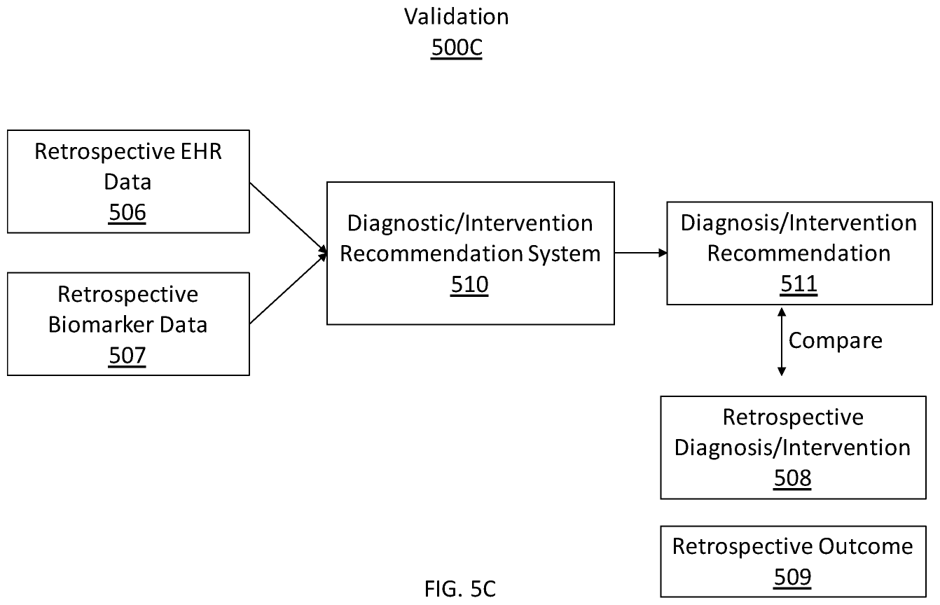

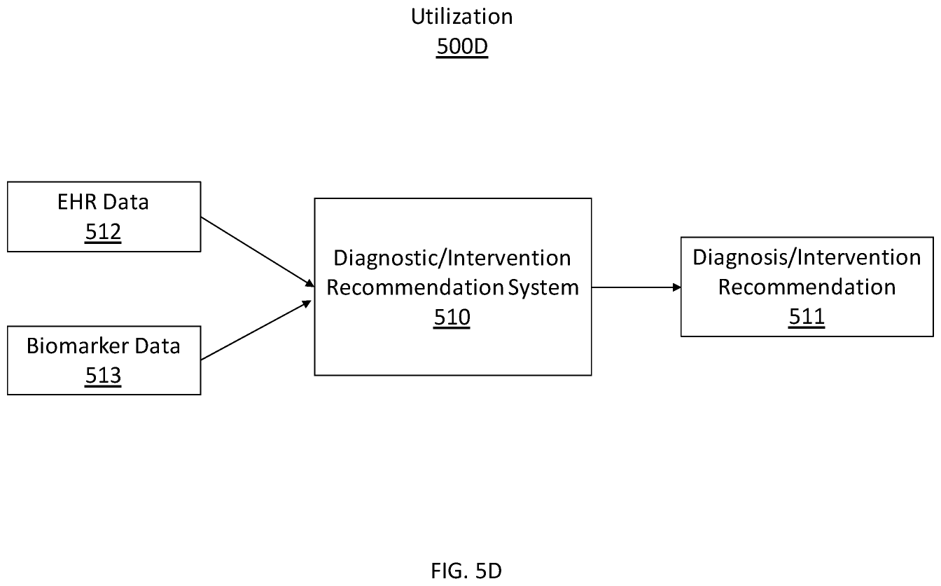

When the diagnostic/intervention recommendation model 304 achieves a threshold level of prediction accuracy (e.g., when the medical outcomes of the subjects for whom diagnostic/intervention recommendations are determined by the model are sufficiently optimized and/or when the fundamental predictive diagnostic metrics of diagnosis recommendations determined by the model are sufficiently optimized), the model is ready for use. To determine when the diagnostic/intervention recommendation model 304 has achieved the threshold level of prediction accuracy sufficient for use, validation of the diagnostic/intervention recommendation model 304 can be performed. Validation of the diagnostic/intervention recommendation model 304 is discussed in further detail below with regard to FIG. 5C.

Once the diagnostic/intervention recommendation model 304 has been validated as having achieved the threshold level of prediction accuracy sufficient for use, in some embodiments, this does not preclude the model from continued training. In fact, in a preferred embodiment, despite validation, the diagnostic/intervention recommendation model 304 continues to be automatically trained such that the set of parameters 306 of the model are automatically and continuously updated, such that the accuracy of the model continues to improve. This automatic and continuous training of the model is discussed in detail below with regard to FIG. 5A.

### II.C.2. Data Store

In some embodiments, the data store 302 stores the training dataset that is used to train the diagnostic/intervention recommendation model 304 as discussed above with regard to the training module 301. As discussed above, the contents of the training dataset depend on the type of the diagnostic/intervention recommendation model 304 being trained. In general, the training dataset comprises a plurality of training samples. Each training sample i from the training dataset is associated with a retrospective subject. Each training sample i that is associated with a retrospective subject comprises EHR data for the retrospective subject and biomarker data for the retrospective subject.

Depending on the type of the diagnostic/intervention recommendation model 304, each training sample i of the training dataset may further comprise additional components. For example, in embodiments in which the diagnostic/intervention recommendation model 304 is learned via supervised learning, each training sample i from the training dataset can further include a retrospective diagnosis/intervention for the retrospective subject associated with the training sample, as well as a known, retrospective medical outcome of the retrospective subject associated with the training sample, following receipt of the retrospective medical diagnosis/intervention. In further or alternative embodiments in which the diagnostic/intervention recommendation model 304 is learned via supervised learning and the diagnostic/intervention recommendation model 304 at least in part comprises a diagnostic recommendation model, each training sample i from the training dataset can also include one or more fundamental predictive diagnostic metrics associated with the retrospective diagnosis for the retrospective subject associated with the training sample.

In some embodiments discussed in detail below with regard to FIGS. 5A and 5C, one or more training samples from the training dataset can be held out from training, and used to validate the diagnostic/intervention recommendation model 304. In alternative embodiments also discussed in detail below with regard to FIGS. 5A and 5C, validation samples other than training samples from the training dataset can be used to validate the diagnostic/intervention recommendation model 304.

Additionally, in some embodiments discussed below with regard to FIGS. 6D-E, the data store 302 does not store training data samples. Rather, in some embodiments, training samples are input directly into the diagnostic/intervention recommendation model 304 without being stored in the data store 302 of the diagnostic/intervention recommendation model 304, at least in part to further comply with possible institutional requirements related to proprietary data and patient information.

### II.C.3. Data Management Module

The data management module 303 generates the training dataset used to train the diagnostic/intervention recommendation model 304. As mentioned above, each training sample i from the training dataset is associated with a retrospective subject. A retrospective subject is any subject for whom at least EHR data and biomarker data are known. Depending on the type of diagnostic/intervention recommendation model 304, a medical diagnosis/intervention, as well as a medical outcome following receipt of the medical diagnosis/intervention, may also be included in the training sample associated with each retrospective subject. Specifically, in embodiments in which the diagnostic/intervention recommendation model 304 is learned via supervised learning, a medical diagnosis/intervention of each retrospective subject, as well as a medical outcome of each retrospective subject following receipt of the medical diagnosis/intervention, is also included in each training sample of the training dataset. As mentioned above, in further or alternative embodiments in which the diagnostic/intervention recommendation model 304 is learned via supervised learning and the diagnostic/intervention recommendation model 304 at least in part comprises a diagnostic recommendation model, one or more fundamental predictive diagnostic metrics associated with the retrospective diagnosis for the retrospective subject can also be included in each training sample of the training dataset.

Data used by the data management module 303 to generate the training dataset can be sourced from a retrospective data source. As discussed in further detail below with regard to FIG. 5A, a retrospective data source can comprise any source of data including publicly-available data, commercially-available data, and/or use data recycled from using the diagnostic/intervention recommendation model 304.

In embodiments in which the training dataset is stored by the data store 302, the data management module 303 stores the generated training dataset in the data store 302. In embodiments in which the diagnostic/intervention recommendation model 304 is also validated, in embodiments in which the training samples include medical diagnoses/interventions of the retrospective subjects, and medical outcomes of the retrospective subjects following receipt of the medical diagnoses/interventions and/or fundamental predictive diagnostic metrics, the data management module 303 can also hold out training samples from the training dataset to be used to validate the diagnostic/intervention recommendation model 304.

### II.C.4. Diagnostic/Intervention Recommendation Model

In various embodiments, the diagnostic/intervention recommendation model 304 is a statistically derived model. In other words, the diagnostic/intervention recommendation model is a non-machine learned model. Such a non-machine learned diagnostic/intervention recommendation model can be configured to receive inputs of EHR data and biomarker data for a subject and to determine a medical diagnosis/intervention recommendation for the subject.

The diagnostic/intervention recommendation model 304 is, in various embodiment, a machine-learned model configured to receive inputs of EHR data and biomarker data for a subject and to determine a medical diagnosis/intervention recommendation for the subject. As discussed above, in general, the diagnostic/intervention recommendation model 304 comprises a function 305 modified by a set of parameters 306 to accurately capture the relationship between independent variables (e.g., EHR and biomarker data) and dependent variables (e.g., diagnosis/intervention recommendations) in the training dataset.

As briefly mentioned above, in some embodiments, the diagnostic/intervention recommendation model 304 comprises a single model configured to determine diagnosis and/or intervention recommendations for a subject. However, in alternative embodiments, the diagnostic/intervention recommendation model 304 can comprise multiple distinct models, each configured to perform a particular task. For example, in one embodiment, the diagnostic/intervention recommendation model 304 can comprise a first model configured to determine diagnoses for subjects, and a second distinct model configured to determine intervention recommendations for subjects. As another example, the diagnostic/intervention recommendation model 304 can comprise a plurality of models, each configured to determine intervention recommendations for subjects diagnosed with a particular condition. In alternative embodiments, the diagnostic/intervention recommendation model 304 can comprise any number of models configured to determine any variation of diagnosis/intervention recommendations for subjects.

As discussed above with regard to the training module 301, the function 305 that in part comprises the diagnostic/intervention recommendation model 304 can comprise a network model NN( ) in some embodiments. In general, a network model comprises a series of nodes arranged in layers. A node may be connected to other nodes through connections each having an associated parameter θ in the set of parameters 306 for the model. A value at one particular node may be represented as a sum of the values of nodes connected to the particular node weighted by the associated parameter mapped by an activation function associated with the particular node. In contrast to the affine function, network models are advantageous because the diagnostic/intervention recommendation model 304 can incorporate non-linearity.

FIG. 4 illustrates an example network model, in accordance with an embodiment. As shown in FIG. 4, the network model NN( ) includes two input nodes at layer l=1, four nodes at layer l=2, two nodes at layer l=3, and one output node at layer l=4. In some embodiments, such as the embodiment depicted in FIG. 4, each node of the network model NN( ) includes one or more functions in a general form of: ƒ(w*x+b), where ƒ( ) is an activation function, x is an input to the node, w is an input-specific weight, and b is an input-specific bias. As described throughout this disclosure, input-specific weights w and biases b are collectively referred to as a parameter θ of the input. The network model NN( ) is associated with a set of 18 different node inputs and thus 18 parameters θ(1), θ(2), . . . , θ(18). The network model NN( ) receives input values x(1) and x(2) for the independent variables of EHR data and biomarker data, and outputs the value NN(x) for the dependent variable of diagnosis/intervention recommendation. In alternative embodiments of a network model, any combination of layers, nodes, functions, and parameters can be included in the network model.

### III. Training, Validation, and Use of Diagnostic/Intervention Recommendation System

FIG. 5A is a block diagram of a system environment 500A in which a diagnostic/intervention recommendation system is trained, validated, and used, in accordance with an embodiment. FIG. 5A includes a training phase 501, a validation phase 502, a use phase 503, a retrospective data store 504, and a prospective data store 505. Thus, FIG. 5A depicts how retrospective and prospective data are used to train, validate, and utilize a diagnostic/intervention recommendation system.

As discussed above with regard to FIG. 3, prior to use of a diagnostic/intervention recommendation system, the system is trained. As shown in FIG. 5A, training of the diagnostic/intervention recommendation system is accomplished using training samples received from the retrospective data source 504. In various embodiments, training samples are continuously received from the retrospective data source 504 such that as the training 501, validation 502, and/or use phases 503 are ongoing, additional training samples are received. The retrospective data source 504 stores training samples that are associated with retrospective subjects for whom at least EHR data and/or biomarker data are known. Depending on the type of the diagnostic/intervention recommendation system, a retrospective medical diagnosis/intervention, and a retrospective medical outcome following receipt of the retrospective medical diagnosis/intervention and/or fundamental predictive diagnostic metrics, may also be included in the training sample associated with each retrospective subject. The data contained in the retrospective data source 504 can include publicly-available data, commercially-available data, use data recycled after use by the diagnostic/intervention recommendation model 304 at the use phase 503, and/or any other source of data.

Following or in conjunction with training, in some embodiments, a diagnostic/intervention recommendation system that is a candidate for use in the use phase 503, can undergo validation in the validation phase 502 to determine whether the candidate system has achieved a threshold level of prediction accuracy and is ready for use. As discussed in further detail below with regard to FIG. 5C, validation of the candidate diagnostic/intervention recommendation system can comprise one or more of internal validation, external validation, and prospective validation. In internal validation, one or more training samples from the retrospective data source 504 can be held out from the training phase 501, and used as validation samples to validate the candidate diagnostic/intervention recommendation system. However, unlike training of the system, validation of the system requires retrospective medical diagnoses/interventions, and retrospective medical outcomes of each retrospective subject and/or fundamental predictive diagnostic metrics, to be known and included in the validation sample associated with the retrospective subject. Thus internal validation of the system, in which training samples from the retrospective data source 504 are held out from the training phase 501 and used as validation samples, can be utilized when the held out training samples from the retrospective data source 504 include retrospective medical diagnoses/interventions, and retrospective medical outcomes of the retrospective subjects and/or fundamental predictive diagnostic metrics.

In contrast, in external validation, samples from a retrospective data source other than the retrospective data source 504 from which training samples are taken, can be used as validation samples to validate the candidate diagnostic/intervention recommendation system. But again, these validation samples also include retrospective medical diagnoses/interventions, and retrospective medical outcomes of the retrospective subjects associated with the validation samples and/or fundamental predictive diagnostic metrics.

And finally, in prospective validation, validation samples used to validate the candidate diagnostic/recommendation system are not obtained from a particular dataset, but rather are obtained in real-time, near real-time, delayed batch or on-demand. For example, as illustrated in FIG. 5A, after the diagnostic/intervention recommendation system uses prospective data 505 to determine a diagnosis/intervention recommendation for a subject during the use phase 503, this prospective data, along with the determined diagnosis/intervention recommendation, and subsequent subject outcome and/or fundamental predictive diagnostic metrics, can be recycled, de-identified, and used as a validation sample to validate the candidate diagnostic/intervention recommendation system.

Once a candidate diagnostic/intervention recommendation system has been validated as having achieved the threshold level of prediction accuracy sufficient for use, the system is ready for the use phase 503. However, in some embodiments, this does not preclude the system from continued training and additional validation to achieve incrementally higher threshold levels of prediction accuracy. In fact, in a preferred embodiment, despite initial validation, the diagnostic/intervention recommendation system continues to undergo training and subsequent validations such that the system is automatically and continuously updated, and the accuracy of the system continues to improve.

Turning to the use phase 503, the diagnostic/intervention recommendation system is used to determine diagnosis/intervention recommendations for subjects associated with data received from the prospective data source 505. The prospective data source 505 is similar to the retrospective data source 504 in that it contains data describing EHR data and biomarker data for subjects. However, unlike some embodiments of the retrospective data source 504, the prospective data source 505 does not include retrospective medical diagnoses/interventions, retrospective medical outcomes for the subjects, or fundamental predictive diagnostic metrics, because these are to be determined by the diagnostic/intervention recommendation system during the use phase 503. The data contained in the prospective data source 505 can include publicly-available data, commercially-available data, data received from a private entity (e.g., a patient care center), and/or any other source of data.

As discussed above, following use of the diagnostic/intervention recommendation system to determine diagnosis/intervention recommendations for subjects during the use phase 503, the independent variables (e.g., EHR data and biomarker data) received by the diagnostic/intervention recommendation system from the prospective data source 505 during the use phase 503, as well as retrospective diagnoses/interventions, and retrospective medical outcomes of the subjects following receipt of the retrospective diagnoses/interventions and/or fundamental predictive diagnostic metrics, can be used as retrospective data to train or validate the system. In other words, prospective data 505 used by the diagnostic/intervention recommendation system during the use phase 503 can be recycled and de-identified to become retrospective data 504 used to train or validate the diagnostic/intervention recommendation system during the training phase 501 or the validation phase 502, respectively. In this way, the diagnostic/intervention recommendation system can be continuously trained and validated.

In various embodiments, the diagnostic/intervention recommendation system can implement federated learning tasks. In various embodiments, the diagnostic/intervention recommendation system implements federated learning during the training phase 501 and/or validation phase 502. In various embodiments, federated learning can be implemented for training and/or validating the diagnostic/intervention recommendation model using multiple datasets without transferring the data to a centralized location (e.g., performing training/validation through a decentralized system). In various embodiments, federated learning can be implemented in the form of federated training where the diagnostic/intervention recommendation model is trained in multiple locations (e.g., decentralized training) irrespective of the validation technique that is performed. In various embodiments, federated learning can be implemented in the form of federated validation where the diagnostic/intervention recommendation model is validated across data from various systems (e.g., decentralized validation). In such embodiments of federated validation, the model can be trained centrally or can be trained in multiple locations.

### III.A. Training

FIG. 5B is a block diagram of a system environment 500B in which a diagnostic/intervention recommendation system 510 is trained via supervised learning, in accordance with an embodiment. As discussed above, training of the diagnostic/intervention recommendation system 510 depends on which type of system the diagnostic/intervention recommendation system 510 comprises. For example, the diagnostic/intervention system 510 can be trained via unsupervised learning or supervised learning. FIG. 5B depicts an embodiment in which the diagnostic/intervention recommendation system 510 is trained via supervised learning. However, the diagnostic/intervention recommendation system 510 can also be trained via unsupervised learning, or according to any other method, in alternative embodiments.

As shown in FIG. 5B, to train the diagnostic/intervention recommendation system 510 via supervised learning, retrospective EHR data 506, retrospective biomarker data 507, a retrospective diagnosis/intervention 508, and a retrospective medical outcome 509 following receipt of the retrospective diagnosis/intervention 508, of a retrospective subject are input into the diagnostic/intervention recommendation system 510. In alternative embodiments in which the diagnostic/intervention recommendation system 510 at least in part comprises a diagnostic recommendation system, fundamental predictive diagnostic metrics associated with the retrospective diagnosis 508 may also be input into the diagnostic/intervention recommendation system 510 during training. Following input of the retrospective EHR data 506, the retrospective biomarker data 507, the retrospective diagnosis/intervention 508, and the retrospective outcome 509 for a retrospective subject into the diagnostic/intervention recommendation system 510, the diagnostic/intervention recommendation system 510 determines and outputs a diagnosis/intervention recommendation 511 based on the retrospective EHR data 506 and the retrospective biomarker data 507. The diagnosis/intervention recommendation 511 output by the diagnostic/intervention recommendation system 510 is not based on the known, retrospective diagnosis/intervention 508 or the known, retrospective outcome 509 input into the diagnostic/intervention recommendation system 510. Rather, the diagnosis/intervention recommendation 511 determined and output by the diagnostic/intervention recommendation system 510 is compared to the retrospective diagnosis/intervention 508 and the retrospective outcome 509. In embodiments in which fundamental predictive diagnostic metrics are input into the diagnostic/intervention recommendation system 510, the diagnosis/intervention recommendation 511 is compared to the retrospective diagnosis/intervention 508 and the fundamental predictive diagnostic metrics.

This comparison of the diagnosis/intervention recommendation 511 determined and output by the diagnostic/intervention recommendation system 510 with the retrospective diagnosis/intervention 508 and the retrospective outcome 509 enables the diagnostic/intervention recommendation system 510 to determine parameters that optimize the medical outcomes of subjects for whom diagnoses/interventions are recommended by the diagnostic/intervention recommendation system 510 as discussed in detail above with regard to FIG. 3. Similarly, comparison of the diagnosis/intervention recommendation 511 with the retrospective diagnosis/intervention 508 and the fundamental predictive diagnostic metrics enables the diagnostic/intervention recommendation system 510 to determine parameters that optimize the fundamental predictive diagnostic metrics for the diagnoses/interventions recommended by the diagnostic/intervention recommendation system 510. In other words, this comparison enables the diagnostic/intervention recommendation system 510 to be trained.

Briefly, in embodiments in which the diagnostic/intervention recommendation system 510 is trained via unsupervised learning, as opposed to supervised learning as discussed above, the retrospective diagnosis/intervention 508, and the retrospective medical outcome 509 of the retrospective subject and/or the fundamental predictive diagnostic metrics, are not used to train the diagnostic/intervention recommendation system 510. Rather, in embodiments in which the diagnostic/intervention recommendation system 510 is trained via unsupervised learning, the retrospective EHR data 506 and the retrospective biomarker data 507 of the retrospective subject are input into the diagnostic/intervention recommendation system 510 without the retrospective diagnosis/intervention 508, and the retrospective medical outcome 509 and/or the fundamental predictive diagnostic metrics.

### III.B. Validation

As discussed above, in some embodiments, following or in conjunction with training, a candidate diagnostic/intervention recommendation system 510 can also undergo validation to determine whether the candidate system has achieved a threshold level of prediction accuracy and is ready for use. FIG. 5C is a block diagram of a system environment 500C in which the diagnostic/intervention recommendation system 510 is validated, in accordance with an embodiment. Briefly, validation of both supervised learning and unsupervised learning systems can occur as described herein with regard to FIG. 5C.

As discussed above with regard to FIG. 5A, validation of the diagnostic/intervention recommendation system 510 can comprise one or more of internal validation, external validation, and prospective validation. The principle difference between these different types of validation is the origin of the validation samples. In internal validation, one or more training samples from a retrospective data source can be held out from training, and used to validate the diagnostic/intervention recommendation system 510. In contrast, in external validation, validation samples from a retrospective data source other than the retrospective data source from which training samples are taken are used to validate the diagnostic/intervention recommendation system 510. And finally, in prospective validation, validation samples used to validate the diagnostic/recommendation system are not obtained from a particular dataset, but rather are obtained in real-time, near real-time, delayed batch or on-demand. However, in each of these types of validation, the validation samples used to validate the diagnostic/intervention recommendation system 510 include retrospective medical diagnoses/interventions, and retrospective medical outcomes of the retrospective subjects associated with the validation samples and/or fundamental predictive diagnostic metrics.

The validation system environment 500C depicted in FIG. 5C applies to each of these different types of validation. As shown in FIG. 5C, to validate the diagnostic/intervention recommendation system 510, retrospective EHR data 506 and retrospective biomarker data 507 for a retrospective subject obtained from any internal, extremal, or prospective source are input into the diagnostic/intervention recommendation system 510. However, unlike in training, a retrospective diagnosis/intervention 508, and a retrospective outcome 509 for the retrospective subject and/or fundamental predictive diagnostic metrics, are not input into the diagnostic/intervention recommendation system 510.

Following input of the retrospective EHR data 506 and the retrospective biomarker data 507 for the retrospective subject into the diagnostic/intervention recommendation system 510, the diagnostic/intervention recommendation system 510 determines and outputs a diagnosis/intervention recommendation 511 based on the retrospective EHR data 506 and the retrospective biomarker data 507. Then, the diagnosis/intervention recommendation 511 output by the diagnostic/intervention recommendation system 510 is compared to the retrospective diagnosis/intervention 508, and the retrospective outcome 509 for the retrospective subject and/or the fundamental predictive diagnostic metrics, that were not input into the diagnostic/intervention recommendation system 510.

The comparison of the diagnosis/intervention recommendation 511 determined and output by the diagnostic/intervention recommendation system 510 with the retrospective diagnosis/intervention 508, and the retrospective outcome 509 and/or the fundamental predictive diagnostic metrics, enables a determination of whether the diagnostic/intervention recommendation system 510 has achieved a threshold level of prediction accuracy. In embodiments in which the diagnosis/intervention recommendation 511 is compared with the retrospective outcome 509, a threshold level of prediction accuracy refers to a threshold medical outcome of the subject. In embodiments in which the diagnosis/intervention recommendation 511 is compared with the fundamental predictive diagnostic metrics, a threshold level of prediction accuracy refers to a threshold fundamental predictive diagnostic metric. In certain embodiments, the diagnostic/intervention recommendation system 510 must be achieve a threshold level of prediction accuracy for more than one subject (e.g., a cohort of subjects).

If the diagnostic/intervention recommendation system 510 is determined to have achieved a threshold level of prediction accuracy based on this comparison, the diagnostic/intervention recommendation system 510 can be considered validated, and is ready for use. In some embodiments, validation of the diagnostic/intervention recommendation system 510 results in an end to training of the diagnostic/intervention recommendation system 510. However, in alternative, preferred embodiments, validation of the diagnostic/intervention recommendation system 510 does not preclude the diagnostic/intervention recommendation system 510 from training, and the diagnostic/intervention recommendation system 510 continues to undergo continuous and automatic training throughout its use.

In embodiments in which the diagnostic/intervention recommendation system 510 is determined to have not achieved a threshold level of prediction accuracy based on the comparison, the diagnostic/intervention recommendation system 510 can be further trained prior to use and validation can be performed again, preferably with new validation samples.

### III.C. Use

Once the diagnostic/intervention recommendation system 510 has been validated as having achieved the threshold level of prediction accuracy, the system is ready to be used. FIG. 5D is a block diagram of a system environment 500D in which the diagnostic/intervention recommendation system 510 is utilized, in accordance with an embodiment. As shown in FIG. 5D and as discussed in detail above, to use the diagnostic/intervention recommendation system 510, EHR data 512 and biomarker data 513 for a subject are input into the diagnostic/intervention recommendation system 510. Unlike some embodiments of the retrospective data used in training and validation, the EHR data 512 and biomarker data 513 are prospective data associated with a subject for whom a retrospective diagnosis/intervention, a retrospective medical outcome, and fundamental predictive diagnostic metrics are not yet known.

Following input of the EHR data 512 and the biomarker data 513 for the subject into the diagnostic/intervention recommendation system 510, the diagnostic/intervention recommendation system 510 determines and outputs a diagnosis/intervention recommendation 511 based on the EHR data 512 and the biomarker data 513. During use, this diagnosis/intervention recommendation 511 is not compared to a retrospective diagnosis/intervention, a retrospective medical outcome for the subject, or fundamental predictive diagnostic metrics because a retrospective diagnosis/intervention, a retrospective medical outcome, and fundamental predictive diagnostic metrics are not yet known. Instead, the diagnosis/intervention recommendation 511 output by the diagnostic/intervention recommendation system 510 is assumed to be sufficiently accurate based on prior training and validation of the diagnostic/intervention recommendation system 510.

However, in some embodiments as discussed above with regard to FIG. 5A, once a retrospective diagnosis/intervention, and a retrospective medical outcome for the subject and/or fundamental predictive diagnostic metrics, are known, the diagnosis/intervention recommendation 511 output by the diagnostic/intervention recommendation system 510, the retrospective diagnosis/intervention, and the retrospective medical outcome and/or the fundamental predictive diagnostic metrics, can be used to train and/or validate the system. In this way, the diagnostic/intervention recommendation system 510 can be continuously and automatically trained and validated throughout use.

### IV. Diagnostic/Intervention Recommendation System Environment

FIG. 6A is a block diagram of a system environment 600A in which a diagnostic/intervention recommendation system 601 operates, in accordance with an embodiment. The system environment 600A shown in FIG. 6A comprises a primary system 602 storing the diagnostic/intervention recommendation system 601, one or more third-party systems 603, and a network 604. In alternative configurations, different and/or additional components may be included in the system environment 600A.

The primary system 602 and the one or more third-party systems 603 are coupled to the network 604 such that the primary system 602 and the one or more third-party systems 603 are in communication with one another via the network 604. The primary system 602 and/or one or more of the third-party systems 603 can comprise a computing system capable of transmitting and/or receiving data via the network 604. Transmission of data over a network can include transmission of data via the internet, wireless transmission of data, non-wireless transmission of data (e.g., transmission of data via ethernet), or any other form of data transmission. In one embodiment, the primary system 602 and/or one or more of the third-party systems 603 can be a conventional computer system, such as a desktop or a laptop computer, or a virtualized machine or container, such as a cloud-enabled virtual machine or docker image, running on a conventional computer system.

In certain embodiments, the primary system 602 and/or one or more third-party systems 603 can be a set of sub-systems (e.g. machines, modules, containers, or microservices) in communication with one another via the network 604, where each sub-system enables one or more of the tasks disclosed herein.

Alternatively, the primary system 602 and/or one or more of the third-party systems 603 can be a device having computer functionality, such as a personal digital assistant (PDA), a mobile telephone, a smartphone or another suitable device. In further embodiments, the primary system 602 and/or one or more of the third party systems 603 can be a non-transitory computer-readable storage medium storing computer program instructions that when executed by a computer processor, cause the computer processor to operate in accordance with the methods discussed throughout this disclosure. In even further embodiments, the primary system 602 and/or one or more of the third-party systems 603 can be cloud-hosted computing systems (e.g., computing systems hosted by Amazon Web Services™ (AWS)).

As shown in FIG. 6A, the diagnostic/treatment system 601 is stored by the primary system 602. Thus the primary system 602 is configured to execute the diagnostic/treatment system 601 as discussed throughout this disclosure. In additional embodiments discussed in further detail below with regard to FIGS. 6C-E, the diagnostic/treatment system 601 can be transmitted between the primary system 602 and one or more of the third-party systems 603 via the network 604. Transmission of the system 601 over a network can include transmission of the system 601 via the internet, wireless transmission of the system 601, non-wireless transmission of the system 6011 (e.g., transmission of the system 601 via ethernet), or any other form of transmission. In such embodiments, one or more of the third-party systems 603 can also be configured to execute the diagnostic/treatment system 601 as discussed throughout this disclosure.

In additional embodiments, one or more of the third-party systems 603 can execute an application allowing the third-party systems 603 to interact with the primary system 602 and/or the diagnostic/treatment system 601 stored by the primary system 602. For example, one or more of the third-party systems 603 can execute a browser application to enable interaction between the third-party systems 603 and the primary system 602 and/or the diagnostic/treatment system 601 via the network 604. In another embodiment, one or more of the third-party systems 603 can interact with the primary system 602 and/or the diagnostic/treatment system 601 through an application programming interface (API) running on native operating systems of the third-party systems 603, such as IOS® or ANDROID™. In one embodiment, one or more of the third-party systems 603 can communicate data to the primary system 602 for use by the diagnostic/treatment system 601 stored by the primary system 602.

In certain embodiments, the primary system 602 and the one or more third-party systems 603 can be remote from one another. In further embodiments the primary system 602 and the one or more third-party systems 603 can be located at one or more patient care centers (e.g., a physician's office, a hospital), clinical laboratories, research laboratories, remote locations, and/or any other sites.

The network 604 can comprise any combination of local area and/or wide area networks, using both wired and/or wireless communication systems. In one embodiment, the network 604 uses standard communications technologies and/or protocols. For example, the network 604 includes communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 604 include multiprotocol label switching (MPLS), transmission control protocol/Internet protocol (TCP/IP), hypertext transport protocol (HTTP), simple mail transfer protocol (SMTP), file transfer protocol (FTP), and voice over internet protocol (VoIP). Data exchanged over the network 604 may be represented using any suitable format, such as hypertext markup language (HTML), extensible markup language (XML), or audio. In some embodiments, all or some of the communication links of the network 604 may be encrypted using any suitable technique or techniques.

As discussed in detail below, FIGS. 6B-E depict various particular embodiments of the system environment 600A, in which the diagnostic/intervention recommendation system 601, training samples, and use data are variably transmitted between the primary system 602 and the one or more third-party systems 603 for training and utilizing of the diagnostic/intervention recommendation system 601.

### IV.A. Training and Utilizing the Diagnostic/Intervention Recommendation Model at a Primary System

Turning first to FIG. 6B, FIG. 6B is a block diagram of a system environment 600B in which the diagnostic/intervention recommendation system 601 operates, in accordance with an embodiment. More specifically, FIG. 6B depicts a system environment 600B in which the diagnostic/intervention recommendation system 601 is both trained and utilized at the primary system 602 using training samples and use data received from a third-party system 603 at the primary system 602 via the network 604. For simplicity, only one third-party system 603 is depicted in FIG. 6B. However, as discussed above with regard to FIG. 6A, the system environment 600A in which the diagnostic/intervention recommendation system 601 operates can comprise one or more third-party systems 603. Thus the third-party system 603 depicted in FIG. 6B should be understood to represent multiple third-party systems 603 in certain embodiments.

As shown in FIG. 6B and as discussed above, the diagnostic/intervention recommendation system 601 is stored by the primary system 602. Furthermore, the primary system 602 and the third-party system 603 are in communication with one another via the network 604.

In the embodiment shown in FIG. 6B, the diagnostic/intervention recommendation system 601 is trained at the primary system 602. To train the diagnostic/intervention recommendation system 601 at the primary system 602, the primary system 602 receives training samples 605 from the third-party system 603. In some embodiments, the primary system 602 receives the training samples 605 from the third-party system 603 via transmission via the network 604. For instance, in some embodiments, the primary system 602 receives the training samples 605 from the third-party system 603 via a browser application via the network 604 and/or via an application programming interface (API) running on a native operating system of the third-party system 603.

Then, using the training samples 605 received from the third-party system 603, the diagnostic/intervention recommendation system 601 determines the parameters of the diagnostic/intervention recommendation system 601 at the primary system 602, as discussed in detail above with regard to FIG. 3. In certain embodiments, the diagnostic/intervention recommendation system 601 can also be validated at the primary system 602 using held-out training samples 605.

In some embodiments, the primary system 602 receives the training samples 605 from multiple distinct third-party systems 603. In such embodiments, one or more of the training samples 605 can be organized according to different data formats. To render these training samples 605 compatible with one another and/or with the diagnostic/intervention recommendation system 601, the one or more training samples 605 organized in different data formats can be transformed into a common data format. In some embodiments, transformation of the one or more training samples 605 in different data formats can be accomplished using a publicly-available data transformation model (e.g., the OMOP Common Data Model). Then, the one or more training samples 605 transformed into the common data format can be merged into a merged training dataset, and this merged training dataset can be used to determine the parameters of the diagnostic/intervention recommendation system 601 at the primary system 602.

In the embodiment shown in FIG. 6B, the diagnostic/intervention recommendation system 601 is also utilized at the primary system 602. To utilize the diagnostic intervention recommendation system 601 at the primary system 602, the primary system 602 receives use data 606 from the third-party system 603. In some embodiments, the primary system 602 receives the use data 606 from the third-party system 603 via transmission via the network 604. For instance, in some embodiments, the primary system 602 receives the use data 606 from the third-party system 603 via a browser application via the network 604 and/or via an application programming interface (API) running on a native operating system of the third-party system 603. In embodiments in which the third-party system 603 comprises multiple third-party systems 603, the primary system 602 can receive use data 606 from one or more of the multiple third-party systems 603. Furthermore, in embodiments in which the third-party system 603 comprises multiple third-party systems 603, the primary system 602 can receive training samples 605 and use data 606 from the same or distinct third-party systems 603.

Then, using the use data 606 received from the third-party system 603, the diagnostic/intervention recommendation system 601 determines a diagnosis/intervention recommendation based on the use data 606 at the primary system 602. In some embodiments, the primary system 602 then provides the diagnosis/intervention recommendation determined by the diagnostic/intervention recommendation system 601 to the third-party system 603 via transmission via the network 604.

### IV.B. Training the Diagnostic/Intervention Recommendation Model at a Primary System and Utilizing the Diagnostic/Intervention Recommendation Model at a Remote Third-Party System

Turning next to FIG. 6C, FIG. 6C is a block diagram of a system environment 600C in which the diagnostic/intervention recommendation system 601 operates, in accordance with an embodiment. More specifically, FIG. 6C depicts a system environment 600C in which the diagnostic/intervention recommendation system 601 is trained at the primary system 602 using training samples received from a third-party system 603 at the primary system 602 via the network 604, but is utilized at a third-party system 603 using use data received at the third-party system 603. For simplicity, only one third-party system 603 is depicted in FIG. 6C. However, as discussed above with regard to FIG. 6A, the system environment 600A in which the diagnostic/intervention recommendation system 601 operates can comprise one or more third-party systems 603. Thus the third-party system 603 depicted in FIG. 6C should be understood to represent multiple third-party systems 603 in certain embodiments.

As discussed above, the diagnostic/intervention recommendation system 601 is stored by the primary system 602. Furthermore, the primary system 602 and the third-party system 603 are in communication with one another via the network 604.

In the embodiment shown in FIG. 6C, the diagnostic/intervention recommendation system 601 is trained at the primary system 602. To train the diagnostic/intervention recommendation system 601 at the primary system 602, the primary system 602 receives training samples 605 from the third-party system 603. In some embodiments, the primary system 602 receives the training samples 605 from the third-party system 603 via transmission via the network 604. For instance, in some embodiments, the primary system 602 receives the training samples 605 from the third-party system 603 via a browser application via the network 604 and/or via an application programming interface (API) running on a native operating system of the third-party system 603.

Then, using the training samples 605 received from the third-party system 603, the diagnostic/intervention recommendation system 601 determines the parameters of the diagnostic/intervention recommendation system 601 at the primary system 602, as discussed in detail above with regard to FIG. 3. In certain embodiments, the diagnostic/intervention recommendation system 601 can also be validated at the primary system 602 using held-out training samples 605.

As discussed above, in some embodiments, the primary system 602 receives the training samples 605 from multiple distinct third-party systems 603. In such embodiments, one or more of the training samples 605 can be organized according to different data formats. To render the training samples 605 in different data formats compatible with one another and/or with the diagnostic/intervention recommendation system 601, the one or more training samples 605 can be transformed into a common data format. In some embodiments, transformation of the one or more training samples 605 organized in different data formats can be accomplished using a publicly-available data transformation model (e.g., the OMOP Common Data Model). Then, the one or more training samples 605 transformed into the common data format can be merged into a merged training dataset, and this merged training dataset can be used to determine the parameters of the diagnostic/intervention recommendation system 601 at the primary system 602.

In the embodiment shown in FIG. 6C, the diagnostic/intervention recommendation system 601 is not utilized at the primary system 602, but rather is utilized at a third-party system 603. The third-party system 603 at which the diagnostic/intervention recommendation system 601 is used can be the same third-party system 603 from which the training samples 605 were received, or a distinct third-party system 603 that did not provide the training samples 605. In embodiments in which the third-party system 603 comprises multiple third-party systems 603, the diagnostic/intervention recommendation system 601 can be utilized at one or more of the multiple third-party systems 603.

As shown in FIG. 6C, the third-party system 603 stores received use data 606. To utilize the diagnostic/intervention recommendation system 601 at the third-party system 603, the primary system 602 provides the diagnostic/intervention recommendation system 601 to the third-party system 603. In certain embodiments, the primary system 602 provides the diagnostic/intervention recommendation system 601 to the third-party system 603 automatically at specified time intervals. In alternative embodiments, the primary system 602 automatically provides the diagnostic/intervention recommendation system 601 to the third-party system 603 in real-time, near real-time, delayed batch or on-demand following identification of the parameters at the primary system 602.

In some embodiments, providing the diagnostic/intervention recommendation system 601 to the third-party system 603 comprises transmitting the diagnostic/intervention recommendation system 601 from the primary system 602 to the third-party system 603 via the network 604. For instance, in some embodiments, providing the diagnostic/intervention recommendation system 601 to the third-party system 603 comprises enabling third-party system 603 to access the diagnostic/intervention recommendation system 601 via a browser application via the network 604 and/or via an application programming interface (API) running on a native operating system of the third-party system 603.

Then, using the use data 606 received at the third-party system 603, the diagnostic/intervention recommendation system 601 determines a diagnosis/intervention recommendation at the third-party system 603. In some embodiments, the third-party system 603 then provides the diagnosis/intervention recommendation determined by the diagnostic/intervention recommendation system 601 to a user (e.g., a subject or a care provider) at the third-party system 603.

In some embodiments, utilizing the diagnostic/intervention recommendation system 601 at individual third-party system 603 using data at the third-party systems 603 can also be advantageous because it alleviates the data privacy and security concerns of transmitting subject data from third-party systems 603 to the primary system 602 for utilizing using the diagnostic/intervention recommendation system 601.

### IV.C. Training and Utilizing the Diagnostic/Intervention Recommendation Model at a Remote Third-Party System

Turning next to FIG. 6D, FIG. 6D is a block diagram of a system environment 600D in which the diagnostic/intervention recommendation system 601 operates, in accordance with an embodiment. More specifically, FIG. 6D depicts a system environment 600D in which the diagnostic/intervention recommendation system 601 is both trained and utilized at a third-party system 603 using training samples and use data received at the third-party system 603. For simplicity, only one third-party system 603 is depicted in FIG. 6D. However, as discussed above with regard to FIG. 6A, the system environment 600A in which the diagnostic/intervention recommendation system 601 operates can comprise one or more third-party systems 603. Thus the third-party system 603 depicted in FIG. 6D should be understood to represent multiple third-party systems 603 in certain embodiments.

As discussed above, the diagnostic/intervention recommendation system 601 is stored by the primary system 602. Furthermore, the primary system 602 and the third-party system 603 are in communication with one another via the network 604.

In the embodiment shown in FIG. 6D, the diagnostic/intervention recommendation system 601 is trained at the third-party system 603. As shown in FIG. 6D, the third-party system 603 stores received training samples 605. To train the diagnostic/intervention recommendation system 601 at the third-party system 603, the primary system 602 provides the diagnostic/intervention recommendation system 601 to the third-party system 603. In some embodiments, providing the diagnostic/intervention recommendation system 601 to the third-party system 603 comprises transmitting the diagnostic/intervention recommendation system 601 from the primary system 602 to the third-party system 603 via the network 604. For instance, in some embodiments, providing the diagnostic/intervention recommendation system 601 to the third-party system 603 comprises enabling third-party system 603 to access the diagnostic/intervention recommendation system 601 via a browser application via the network 604 and/or via an application programming interface (API) running on a native operating system of the third-party system 603.

Then, using the training samples 605 received at the third-party system 603, the diagnostic/intervention recommendation system 601 determines the parameters of the diagnostic/intervention recommendation system 601 at the third-party system 603, as discussed in detail above with regard to FIG. 3. In certain embodiments, the diagnostic/intervention recommendation system 601 can also be validated at the third-party system 603 using held-out training samples 605.

Finally, in certain embodiments, after the diagnostic/intervention recommendation system 601 has been trained and/or validated at the third-party system 603, the diagnostic/intervention recommendation system 601, now comprising the updated parameters, can be transmitted back to the primary system 602 from the third-party system 603 via the network 604. In some embodiments, the third-party system 603 automatically provides the diagnostic/intervention recommendation system 601 with the identified plurality of parameters to the primary system 602 at specified time intervals. In alternative embodiments, the third-party system 603 automatically provides the diagnostic/intervention recommendation system 601 with the identified plurality of parameters to the primary system 602 in real-time, near real-time, delayed batch or on-demand following identification of the parameters at the third-party system 603. In embodiments in which the third-party system 603 comprises multiple third-party systems 603, the diagnostic/intervention recommendation system 601 can be trained at one or more of the multiple third-party systems 603.

In some embodiments, this method of training and validating the diagnostic/intervention recommendation system 601 in which the diagnostic/intervention recommendation system 601 is transmitted to individual third-party systems 603 for training and validation using data at the third-party systems 603 can be advantageous because it enables cross-institutional data sharing by alleviating the data privacy and security concerns of transmitting data from third-party systems 603 to the primary system 602 for use in training and validating the diagnostic/intervention recommendation system 601. By enabling this cross-institutional data sharing, the diagnostic/intervention recommendation system 601 can be trained on a wide variety and a large quantity of data at multiple distinct third-party systems 603, thereby enabling the diagnostic/intervention recommendation system 601 to be rapidly optimized to determine diagnosis/intervention recommendations with high accuracy.

In the embodiment shown in FIG. 6D, the diagnostic/intervention recommendation system 601 is also utilized at a third-party system 603. The third-party system 603 at which the diagnostic/intervention recommendation system 601 is used can be the same third-party system 603 at which the diagnostic/intervention recommendation system 601 was trained, or a distinct third-party system 603 at which the diagnostic/intervention recommendation system 601 was not trained. In embodiments in which the third-party system 603 comprises multiple third-party systems 603, the diagnostic/intervention recommendation system 601 can be utilized at one or more of the multiple third-party systems 603.

As shown in FIG. 6D, the third-party system 603 stores received use data 606. To utilize the diagnostic/intervention recommendation system 601 at the third-party system 603, the primary system 602 provides the diagnostic/intervention recommendation system 601 to the third-party system 603. As mentioned above, in some embodiments, providing the diagnostic/intervention recommendation system 601 to the third-party system 603 comprises transmitting the diagnostic/intervention recommendation system 601 from the primary system 602 to the third-party system 603 via the network 604. For instance, in some embodiments, providing the diagnostic/intervention recommendation system 601 to the third-party system 603 comprises enabling third-party system 603 to access the diagnostic/intervention recommendation system 601 via a browser application via the network 604 and/or via an application programming interface (API) running on a native operating system of the third-party system 603.

Then, using the use data 606 received at the third-party system 603, the diagnostic/intervention recommendation system 601 determines a diagnosis/intervention recommendation at the third-party system 603. In some embodiments, the third-party system 603 then provides the diagnosis/intervention recommendation determined by the diagnostic/intervention recommendation system 601 to a user (e.g., a subject or a care provider) at the third-party system 603.

As similarly described above, utilizing the diagnostic/intervention recommendation system 601 at individual third-party system 603 using data at the third-party systems 603 can also be advantageous because it also alleviates the data privacy and security concerns of transmitting subject use data from third-party systems 603 to the primary system 602 utilizing using the diagnostic/intervention recommendation system 601.

### IV.D. Training the Diagnostic/Intervention Recommendation Model at a Remote Third-Party System and Utilizing the Diagnostic/Intervention Recommendation Model at a Primary System

Turning next to FIG. 6E, FIG. 6E is a block diagram of a system environment 600E in which the diagnostic/intervention recommendation system 601 operates, in accordance with an embodiment. More specifically, FIG. 6E depicts a system environment 600E in which the diagnostic/intervention recommendation system 601 is trained at a third-party system 603 using training samples received at the third-party system 603 and utilized at the primary system 602 using use data received from a third-party system 603. For simplicity, only one third-party system 603 is depicted in FIG. 6E. However, as discussed above with regard to FIG. 6A, the system environment 600A in which the diagnostic/intervention recommendation system 601 operates can comprise one or more third-party systems 603. Thus the third-party system 603 depicted in FIG. 6E should be understood to represent multiple third-party systems 603 in certain embodiments.

As discussed above, the diagnostic/intervention recommendation system 601 is stored by the primary system 602. Furthermore, the primary system 602 and the third-party system 603 are in communication with one another via the network 604.

In the embodiment shown in FIG. 6E, the diagnostic/intervention recommendation system 601 is trained at the third-party system 603. As shown in FIG. 6E, the third-party system 603 stores received training samples 605. To train the diagnostic/intervention recommendation system 601 at the third-party system 603, the primary system 602 provides the diagnostic/intervention recommendation system 601 to the third-party system 603. In some embodiments, providing the diagnostic/intervention recommendation system 601 to the third-party system 603 comprises transmitting the diagnostic/intervention recommendation system 601 from the primary system 602 to the third-party system 603 via the network 604. For instance, in some embodiments, providing the diagnostic/intervention recommendation system 601 to the third-party system 603 comprises enabling third-party system 603 to access the diagnostic/intervention recommendation system 601 via a browser application via the network 604 and/or via an application programming interface (API) running on a native operating system of the third-party system 603.

Then, using the training samples 605 received at the third-party system 603, the diagnostic/intervention recommendation system 601 determines the parameters of the diagnostic/intervention recommendation system 601 at the third-party system 603, as discussed in detail above with regard to FIG. 3. In certain embodiments, the diagnostic/intervention recommendation system 601 can also be validated at the third-party system 603 using held-out training samples 605.

Finally, in certain embodiments, after the diagnostic/intervention recommendation system 601 has been trained and/or validated at the third-party system 603, the diagnostic/intervention recommendation system 601, now comprising the updated parameters, can be transmitted back to the primary system 602 from the third-party system 603 via the network 604. In some embodiments, the third-party system 603 automatically provides the diagnostic/intervention recommendation system 601 with the identified plurality of parameters to the primary system 602 at specified time intervals. In alternative embodiments, the third-party system 603 automatically provides the diagnostic/intervention recommendation system 601 with the identified plurality of parameters to the primary system 602 in real-time, near real-time, delayed batch or on-demand following identification of the parameters at the third-party system 603. In embodiments in which the third-party system 603 comprises multiple third-party systems 603, the diagnostic/intervention recommendation system 601 can be trained at one or more of the multiple third-party systems 603.

In some embodiments, this method of training and validating the diagnostic/intervention recommendation system 601 in which the diagnostic/intervention recommendation system 601 is transmitted to individual third-party systems 603 for training and validation using data at the third-party systems 603 can be advantageous because it enables cross-institutional data sharing by alleviating the data privacy and security concerns of transmitting data from third-party systems 603 to the primary system 602 for use in training and validating the diagnostic/intervention recommendation system 601. By enabling this cross-institutional data sharing, the diagnostic/intervention recommendation system 601 can be trained on a wide variety and a large quantity of data at multiple distinct third-party systems 603, thereby enabling the diagnostic/intervention recommendation system 601 to be rapidly optimized to determine diagnosis/intervention recommendations with high accuracy.

In the embodiment shown in FIG. 6E, the diagnostic/intervention recommendation system 601 is utilized at the primary system 602. To utilize the diagnostic intervention recommendation system 601 at the primary system 602, the primary system 602 receives use data 606 from a third-party system 603. In some embodiments, the primary system 602 receives the use data 606 from the third-party system 603 via transmission via the network 604. For instance, in some embodiments, the primary system 602 receives the use data 606 from the third-party system 603 via a browser application via the network 604 and/or via an application programming interface (API) running on a native operating system of the third-party system 603. In embodiments in which the third-party system 603 comprises multiple third-party systems 603, the primary system 602 can receive use data 606 from one or more of the multiple third-party systems 603. Furthermore, in embodiments in which the third-party system 603 comprises multiple third-party systems 603, the primary system 602 can receive use data 606 from the same third-party system 603 at which the diagnostic/intervention recommendation system 601 was trained, or from a distinct third-party system 603 at which the diagnostic/intervention recommendation system 601 was not trained.

Then, using the use data 606 received from the third-party system 603, the diagnostic/intervention recommendation system 601 determines a diagnosis/intervention recommendation based on the use data 606 at the primary system 602. In some embodiments, the primary system 602 then provides the diagnosis/intervention recommendation determined by the diagnostic/intervention recommendation system 601 to the third-party system 603 via transmission via the network 604.

### V. Indication for Medical Interventions

In embodiments in which the diagnostic/intervention recommendation system is configured to recommend medical interventions for subjects, the intervention recommendation system can also be used to generate datasets that provide evidence in support of new or more specific indications for the medical interventions. Specifically, the intervention recommendation system can be used to identify new or more highly-specified cohorts of subjects that are likely to respond positively, respond negatively, or not respond to specific medical interventions. These new or more specific indications for medical interventions can be supported by datasets including data describing the subjects of the cohorts. Such evidence for medical interventions can then be used as justification for clearance/approval by regulatory bodies (e.g., FDA). For example, such evidence for medical interventions can be used to justify clearance of diagnostics to guide use of the medical intervention or to justify approval of combinations of new and existing medical interventions, including approval of drug labels providing indications for drug use.

In general, as discussed above, the intervention recommendation system uses EHR data and/or biomarker data for subjects to determine medical interventions for the subjects. This EHR data and/or biomarker data can be received by the intervention recommendation system from a multitude of distinct third-party sources (e.g., hospitals). As mentioned above, this data received from multiple distinct sources can be represented in multiple, distinct data formats that are incompatible and incomparable with one another. One advantage to using the intervention recommendation system to determine medical interventions based on the EHR and/or biomarker data is that the system transforms the data into a common data format such that data received from multiple different sources can be automatically compared. Additionally, the intervention recommendation system is able to accomplish this data management while remaining HIPAA compliant.

Following determination of the medical intervention for subjects based on their EHR data and/or biomarker data by the intervention recommendation system, the subjects can be provided either with a standard-of-care medical intervention or with the medical intervention determined by the system. For example, in some embodiments, subjects for whom the intervention recommendation system provides no recommendation may be provided with the standard-of-care medical intervention. Then, following provision of medical interventions, medical outcomes of the subjects can be identified. For a particular medical intervention, the EHR data and/or biomarker data transformed into the common data format by the intervention recommendation system, as well as the medical outcomes of subjects provided with the medical intervention, can be collected and used to generate a dataset that provides evidence in support of a new or more specific indication for the medical intervention. The indication can comprise at least one of EHR data and biomarker data for subjects, and is identified based on the medical outcomes for the subjects that received the medical intervention. For example, a positive indication for a medical intervention A (e.g., affirmation of safe and efficacious use of the medical intervention A) can comprise EHR data and biomarker data for subjects that experienced positive medical outcomes following the receipt of the medical intervention A. As another example, a negative indication for a medical intervention B (e.g., a warning to not use the medical intervention B) can comprise EHR data and biomarker data for subjects that experienced negative medical outcomes following the receipt of the medical intervention B.

Therefore, the intervention recommendation system enables a highly-scaled, real-time, near real-time, delayed batch or on-demand method for determining new or more specific indications for medical interventions, including new medical intervention recommendations and standard-of-care medical interventions. In other words, the intervention recommendation system enables a highly-scaled, real-time, near real-time, delayed batch or on-demand method for determining new or more specific medical interventions for cohorts of subjects identified based on one or more of EHR data and biomarker data.

Generation of a dataset that provides evidence in support of an indication for a medical intervention can be best illustrated in an example. FIGS. 7A and 7B depict example datasets 700A and 700B that provide evidence in support of indications for corticosteroid intervention, in accordance with an embodiment. The datasets 700A and 700B provide the following data points for each of subjects 1-16: a medical diagnosis, biomarker data, a medical intervention recommendation provided by an intervention recommendation system (and, in cases in which no recommendation is provided by the system, a standard-of-care medical intervention), and a medical outcome of the subject following administration of the medical intervention.

The medical diagnosis for a subject comprises a binary indication of whether the subject is septic. For example, as seen in FIG. 7A, the medical diagnosis for subject 1 is sepsis (e.g., “sepsis +”). On the other hand, the medical diagnosis for subject 4 is not sepsis (e.g., “sepsis −”). In the example provided herein, the medical diagnoses for subjects 1-16 are determined by a diagnostic recommendation system, as discussed throughout this disclosure. In alternative embodiments, the medical diagnoses can be determined according to standard-of-care protocols.

In some embodiments, after diagnosis of a subject with sepsis, the subject is treated with fluids and antibiotics to reverse sepsis and to prevent sepsis from progressing to septic shock. If a subject does progress into septic shock, the subject can receive vasopressors and/or other treatments to stabilize the subject's blood pressure. If a subject with septic shock does not achieve a stable blood pressure, the subject is considered to be in vasopressor refractory shock. At this point, standard-of-care sepsis management guidelines provide weak guidance as to whether or not corticosteroids should be administered to a subject in refractory shock. Specifically, certain standard-of-care sepsis management guidelines state that “giving steroids or not are both acceptable because the evidence for doing either is weak.” As a result of this weak guidance, standard-of-care medical interventions (e.g., administration of corticosteroids or no administration of corticosteroids), and thus medical outcomes following administration of these medical interventions, vary from subject to subject.

As an alternative to proceeding according to this weak guidance, an intervention recommendation system can be employed to provide a recommendation as to whether a subject in refractory shock should receive corticosteroid intervention. In some embodiments, such as the embodiment discussed in this example, the intervention recommendation system can provide a recommendation as to whether a subject should receive corticosteroid intervention based on biomarker data determined for the subject. The biomarker data for each subject 1-16 in the example datasets 700A and 700B comprises an indication of a subject subtype (e.g., subtype A, B, or C), based on genetic expression in the subject. For example, as shown in FIG. 7A, the biomarker data for subject 1 indicates that subject 1 is of subtype A. As another example, the biomarker data for subject 3 indicates that subject 3 is of subtype C.

Based on the biomarker data for a subject, the intervention recommendation system determines a medical intervention recommendation for the subject as discussed throughout this disclosure. For example, as shown in FIG. 7A, the medical intervention recommendation determined for subject 1 by the intervention recommendation system is no steroids. As another example, no intervention recommendation was determined for subject 2 by the intervention recommendation system. Note that no biomarker data and no medical intervention recommendations are determined for subjects that are determined to not be septic (e.g., subject 4).

Medical interventions are administered to the subjects according to the recommendations determined by the system. As mentioned above, in embodiments in which no intervention recommendation is determined for a subject by the intervention recommendation system, the standard-of-care medical intervention is administered to the subject. For example, because no medical intervention recommendation was determined for subject 3 by the intervention recommendation system, the standard-of-care medical intervention of steroids was administered to subject 3.

Finally, the medical outcome of each subject is recorded following administration of the medical intervention. In the example provided in FIGS. 7A-B, the medical outcome of a subject comprises a binary indication of whether a subject survived or did not survive following administration of the medical intervention. However, as discussed in further detail below in Section VIII.A., the medical outcome of a subject can comprise any of a diverse plurality of medical outcomes. For example, in alternative embodiments, the medical outcome of a subject can comprise an indication of ventilator dependency, Extra Corporeal Membrane Oxygenation (ECMO) dependency, or duration of stay at a patient care center.

The biomarker data, the medical intervention recommendation (and, in cases in which no recommendation is provided by the system, the standard-of-care medical intervention), and the medical outcome for each subject provide evidence in support of indications for corticosteroid intervention. In particular, in this example, the biomarker data, the medical intervention recommendations (or standard-of-care medical interventions), and the medical outcomes for the subjects provide evidence in support of indications for corticosteroid intervention for subjects diagnosed with sepsis, depending on the subjects' biomarker data. Specifically, the dataset 700A depicted in FIG. 7A provides evidence in support of non-administration of corticosteroid therapy in subjects diagnosed with sepsis that are of subtype A. Similarly, the dataset 700B depicted in FIG. 7B provides evidence in support of non-administration of corticosteroid therapy in subjects diagnosed with sepsis that are of subtype A, but also evidence in support of administration of corticosteroid therapy in subjects diagnosed with sepsis that are of subtype C.

In addition to providing evidence in support of indications for corticosteroid therapy, the datasets 700A and 700B also depict improvement of the medical intervention recommendation system, and as a result, improvement of the medical intervention recommendations and corresponding subject medical outcomes, and improvement of the evidence in support of the medical intervention indications. For instance, in the example provided in FIGS. 7A and 7B, version 1.0 of the medical intervention recommendation system is used to determine medical intervention recommendations for subjects 1-8 in FIG. 7A. This version 1.0 of the intervention recommendation system is trained and optimized to improve the medical outcomes depicted in the dataset 700A, according to methods discussed throughout this disclosure. The trained and optimized version of the system is version 1.1 of the intervention recommendation system. Version 1.1 of the intervention recommendation system is subsequently used to determine medical intervention recommendations for subjects 9-16 in FIG. 7B.

For direct comparison of the improvement in medical intervention recommendations and corresponding subject medical outcomes enabled by the improved version of the intervention recommendation system, subjects 1-4, subjects 5-8, subjects 9-12, and subjects 13-16, respectively, have the same medical diagnosis and the same biomarker data. For example, subjects 2, 6, 10, and 14 all have the same medical diagnosis recommendation of sepsis+ and the same biomarker data of subtype A. As shown in FIG. 7A, because the same version of the medical intervention recommendation system is used to determine medical intervention recommendations for subjects 1-8, the system recommends the same medical interventions for subjects 1-8 with the same medical diagnosis and biomarker data. For example, version 1.0 of the medical intervention recommendation system provides no medical intervention recommendation to both subjects 3 and 7, despite the fact that both subjects survived when administered steroids according to standard-of-care sepsis management guidelines. However, after being trained on the dataset 700A to optimize subject medical outcome, version 1.1 of the medical intervention recommendation system learns to recommend administration of corticosteroids to subjects 11 and 15, as shown in FIG. 7B. Therefore, as shown in FIGS. 7A and 7B, as the medical intervention recommendation system is used and trained, the system's recommendations improve, resulting in improved subject medical outcomes and improved datasets providing indications for medical interventions.

For example, as described above, unlike the dataset 700A, due to updating of the medical intervention recommendation system from version 1.0 to version 1.1, the dataset 700B, generated in part by version 1.1 of the medical intervention recommendation system, provides evidence in support of administration of corticosteroid therapy in subjects diagnosed with sepsis that are of subtype C. Therefore, the dataset 700B provides improved evidence in support of positive and negative indications for corticosteroid intervention relative to the dataset 700A, because the dataset 700B is generated in part by an updated version (version 1.1) of a diagnostic/intervention recommendation system, compared to the dataset 700A, which is generated in part by a less-trained version (version 1.0) of the diagnostic/intervention recommendation system.

The example datasets 700A and 700B provided in FIGS. 7A-B are relatively small datasets with a limited number of variables. These small datasets and limited quantities of variables enable a rough determination of indications for intervention of sepsis with corticosteroids. However, in alternative embodiments, a larger dataset with a greater quantity and variety of variables can provide a more accurate and precise indication for intervention of sepsis with corticosteroids. Generation of these larger datasets used to identify intervention indications requires advanced data processing capabilities at a high level of efficiency, a large amount of data storage/memory, and the ability to capture a large quantity and diversity of patient data from multiple, incompatible third-party sources. These requirements cannot be efficiently fulfilled by even the most skilled individuals in the field, but can be much more easily satisfied by the diagnostic/intervention recommendation system configured to receive, output, and store a large quantity of complex data at high speeds and with great accuracy, as described throughout this disclosure. As discussed in detail below, this ability to quickly and accurately generate, organize, and store large quantities of complex data is one of the primary advantages provided by the diagnostic/intervention recommendation system.

### VI. Data Integrity and Validation

In various embodiments, the diagnostic/intervention recommendation system (e.g., diagnostic/intervention recommendation system described in any of FIGS. 1-3, 5A-5D, and 6A-6E) performs data integrity checks and validations to prevent the use of tampered data and to enable retrospective auditing, if needed. For example, the diagnostic/intervention recommendation system can perform data integrity checks on data received from third parties, data that has been persistently stored by the diagnostic/intervention recommendation system and to be subsequently used, training data to be used to train a diagnostic/intervention recommendation model, data to be inputted into a trained diagnostic/intervention recommendation model, or data pertaining to diagnostic/intervention recommendations outputted by a diagnostic/intervention recommendation model. In particular embodiments, the diagnostic/intervention recommendation system can perform data integrity checks on patient EHR data and/or biomarker data obtained from patients. In various embodiments, the data integrity check, data validation, and auditing processes described herein can be performed by the data management module 303 of the diagnostic/intervention recommendation system 300 (see FIG. 3).

In various embodiments, the diagnostic/intervention recommendation system receives a message (e.g., a message from a third party or an internal message generated by a subsystem or module of the diagnostic/intervention recommendation system intended for another subsystem or module). Such messages can be asynchronously communicated to the diagnostic/intervention recommendation system (e.g. polling queueing services like SQS, RabbitMQ, etc., polling a database, etc.) or synchronous (e.g. http requests, TCP/UDP packets, etc.). In response to the message, the diagnostic/intervention recommendation system performs a designated task and can further generate outgoing messages to trigger subsequent events. Example designated tasks can include loading data into a database, determining which models/function should be triggered, or executing a model/function. Thus, the diagnostic/intervention recommendation system can perform data integrity checks, data validation, and/or auditing processes on the data involved in a task.

In various embodiments, a message includes two elements: 1) a message envelope and 2) a message payload. The message envelope can contain meta-data about the message (e.g., timestamps, one-way cryptographic hashes of the message content (which can optionally be hashed with an additional, secret salt to verify origin of message), the originating micro-service, organization or entity the message is associated with when used in a multi-tenant architecture).

The message payload, in various embodiments, contains the relevant data for which the task is to be performed. In various embodiments, the message payload contains a reference as to the location of the relevant data such that the relevant data can be accessed for performing the task. For example, the relevant data may be persisted in a permanent or semi-permanent storage medium (e.g., flat file, AWS S3, minio, glusterfs). In various embodiments, the message payload is encrypted using one or more encryption mechanisms. For example, a message's payload may be stored in AWS S3 using the AWS KMS encryption mechanism. As another example, the message payload can be encrypted with a symmetric key encryption mechanism, e.g. AES-256, and the encryption key can be included in the message payload with the reference to the persisted message payload path. In this scenario when symmetric key encryption is implemented, a new, random encryption key can be generated for each message. This encryption key is then included within the message so that the diagnostic/intervention recommendation system may decrypt and read the message payload. This encryption key can also be used by the diagnostic/intervention system for subsequently tracking/auditing the performed task to ensure that the task was appropriately performed.

In various embodiments, a cryptographic hash of the message payload (e.g., encrypted payload) can be stored such that the cryptographic hash can be later verified to determine whether any tampering has occurred with the message payload. For example, the cryptographic hash of the message payload can be stored in the message envelope. In various embodiments, the cryptographic hash is a one-way cryptographic hash. Any changes to the message payload would result in a different cryptographic hash that would not match with the cryptographic hash stored in the message envelope.

In various embodiments, the diagnostic/intervention system validates the data to ensure that it is within tolerances. For example, the diagnostic/intervention system can validate patient EHR data and/or patient biomarker data. Therefore, patient EHR data and/or patient biomarker data that is beyond tolerances (e.g., unrealistic data such as a heart rate above 300 beats per minute, negative age) can be identified as erroneous. Erroneous data can be corrected or removed from the data such that the performance of the task is not negatively affected due to the erroneous data.

In various embodiments, to validate the data in the message payload, the diagnostic/intervention system compares portions of the encrypted message. For example, the one-way cryptographic hashes of the message payload are compared to the hashes contained within the message envelope. This ensures no data tampering has occurred, and that the message originated from within the system.

In various embodiments, the diagnostic/intervention recommendation system tracks and stores details about each performed tasked, including any events that led to errors and the associated error details. For example, this event tracking stores one or more of dates, message envelope information (including the one-way cryptographic hashes of each message), message payload, the symmetric key encryption, and persisted message payload information in some storage medium (flat files, database, etc.). By storing meta-data about each task, the diagnostic/intervention recommendation system can, at a later timepoint, perform audits to ensure that the performed tasks were correct. For example, the diagnostic/intervention recommendation system can validate timestamps, validate message integrity, track any errors that may have been encountered. This event tracking/auditing mechanism also allows for replaying received messages, so long as the message payload was persisted within some storage medium. This allows retroactive debugging of errors, verifying bug fixes that may have been associated with a specific error, etc.

### VII. System Architecture and Self-Scaling Implementation

In various embodiments, the diagnostic/intervention system includes a self-scaling architecture that enables the for scaling up or down operations to more efficiently perform the methods described herein. The scaling up or down of operations can be triggered by the diagnostic/intervention system in response to a computational metric being met. Example computational metrics include the CPU utilization exceeding or falling below a threshold value, memory utilization exceeding or falling below a specified value, number of TCP connections exceeding or falling below a specified value, the number of pending computational messages received by the diagnostic/intervention system (messages that are in the queue/database/that have not yet been consumed by the diagnostic/intervention system) exceeding or falling below a specified value.

In one embodiment, when the diagnostic/intervention system implements a diagnostic/intervention recommendation model, the diagnostic/intervention system monitors computational operations for satisfying a computational metric. In response to one of the aforementioned computational metrics being met, the diagnostic/intervention system either scales up or scales down operations to more efficiently implement the diagnostic/intervention recommendation model. For example, the implementation of the diagnostic/intervention recommendation model can result in the CPU utilization and/or memory utilization exceeding a threshold value. In this example, the diagnostic/intervention system can scale up operations, as described in further detail below, for implementing the diagnostic/intervention recommendation model.

In various embodiments, the diagnostic/intervention system is designed to run directly on hardware (e.g., “bare metal”). In such embodiments, the scaling mechanism can increase or decrease the number of instances of a particular service (in this case, a process on the computer).

In various embodiments, the diagnostic/intervention system is designed to run as a virtualized computer (e.g., an EC2 AMI, VMWare image, etc.). In such embodiments that employ virtualized instances, scaling up refers to providing additional virtual instances of the computer hosting the service which is being scaled. Conversely, scaling down refers to removing or putting virtual instances on standby so as to consume fewer resources.

In various embodiments, the diagnostic/intervention system is designed to run as a docker (or any other containerization solution) container. In such embodiments that employ a docker infrastructure, scaling up refers to increasing the number of containers and/or nodes associated with a particular service. Conversely, scaling down refers to reducing the number of containers and/or nodes associated with the particular service. When using kubernetes and docker, the scaling mechanism may be handled by horizontal pod autoscaler (HPA), or custom scaling mechanisms based on any/all collected metrics.

### VIII. Examples

In clinical settings, the ability to quickly and accurately recommend diagnoses and interventions for subjects is crucial as delay in the ability to make clinical decisions and lack of effective interventions can negatively affect a subject's outcome. This is especially true in acute care situations. As discussed in detail throughout this disclosure, the diagnostic/intervention recommendation system can be trained to optimize for a variety of performance metrics, including subject medical outcomes and, in embodiments in which the diagnostic/intervention recommendation system at least in part comprises a diagnostic recommendation system, fundamental predictive diagnostic metrics. Furthermore, the diagnostic/intervention recommendation system can be trained to optimize for any weighted combination of these performance metrics. Thus, when compared to current standards of care, use of the diagnostic/intervention recommendation system in a clinical setting is expected to provide marked improvement according to this wide variety of performance metrics.

To demonstrate the superior capabilities of the diagnostic/intervention recommendation system disclosed herein relative to alternative solutions, such as standard-of-care solutions, for determining diagnosis/intervention recommendations, prospective experiments comparing performance metrics for the diagnostic/intervention recommendation system and alternative solutions will be performed. Based on known data describing the performance of alternative solutions, and based on preliminary data describing the performance of the diagnostic/intervention recommendation system disclosed herein, the diagnostic/intervention recommendation system is expected to exceed performance of alternative solutions according to performance metrics of subject medical outcomes, and, in embodiments in which the diagnostic/intervention recommendation system at least in part comprises a diagnostic recommendation system, fundamental predictive diagnostic metrics.

As detailed below, subject medical outcome metrics include: reduced morbidity of subjects, reduced mortality of subjects, increased quantity of intervention-free days of subjects, reduced time to provide medical diagnosis recommendations and/or medical intervention recommendations to the subjects, reduced cost of stay of subjects at patient care centers at which the subjects receive medical diagnosis recommendations and/or intervention recommendations from the system, reduced length of stay of subjects at patient care centers at which subjects receive the medical diagnosis recommendations and/or intervention recommendations from the system, reduced quantity of adverse events of subjects, reduced rate of adverse events of subjects, improved patient quality scores of subjects, improved patient care center quality scores for a patient care centers at which subjects receive the medical diagnosis recommendations and/or intervention recommendations from the system, improved patient satisfaction with a patient care center at which the subject receives the medical diagnosis recommendations and/or intervention recommendations form the system, increased patient throughput at patient care centers at which subjects receive the medical diagnosis recommendations and/or intervention recommendations from the system, and increased revenue of patient care centers at which subjects receive the medical diagnosis recommendations and/or intervention recommendations from the system. Furthermore, as detailed below in Section VIII.A.2., fundamental predictive diagnostic metrics include: sensitivity, specificity, negative predictive value, positive predictive value, accuracy, area under a ROC (receiver operating characteristic) curve, area under a precision-recall curve, and calibration. Definitions and examples for each of these performance metrics are provided below in Sections VIII.A.1. and VIII.A.2.

As mentioned above, the diagnosis/intervention recommendations determined by the diagnostic/intervention recommendation system are expected to exceed performance of diagnoses/interventions identified by alternative solutions according to the above performance metrics. Additionally, as discussed in detail throughout this disclosure, the diagnostic/intervention recommendation system itself improves as the system undergoes training and its parameters are updated. Thus, elevated performance of the diagnostic/intervention recommendation system relative to alternative solutions as described above applies not only to systems with different architectures (e.g., functions), but also applies to prior versions or iterations of the diagnostic/intervention recommendation system itself. In other words, elevated performance of the diagnostic/intervention recommendation system relative to alternative solutions as described above also applies to diagnostic/intervention recommendation systems comprising the same architecture but different parameters.

As discussed above, at a high level, use of the diagnostic/intervention recommendation system to determine diagnosis/intervention recommendations for subjects is expected to improve patient outcomes and reduce both the time and the cost of diagnosing and/or treating a subject. These marked improvements afforded by the diagnostic/intervention recommendation system are enabled by the ability of the system to efficiently and accurately receive, store, and process a large quantity of data from a wide-range of disparate—and oftentimes incompatible—sources. Additionally, the diagnostic/intervention recommendation system is able to accomplish this data management all while preserving patient privacy and remaining HIPAA compliant. Based on these capabilities, the diagnostic/intervention recommendation system is expected to operate at a higher level of performance than even the best human care providers and to provide a vital improvement to current standards of care.

### VIII.A. Performance Metric Definitions

As discussed in detail above, in prospective examples, the diagnostic/intervention recommendation system is expected to exceed performance of alternative diagnostic/intervention recommendation solutions according to a wide variety of performance metrics, including subject medical outcomes and, in embodiments in which the diagnostic/intervention recommendation system at least in part comprises a diagnostic recommendation system, fundamental predictive diagnostic metrics. Furthermore, the diagnostic/intervention recommendation system is expected to exceed performance of alternative diagnostic/intervention recommendation solutions according to any weighted combination of the performance metrics described herein. Definitions and examples of these performance metrics are provided below.

### VIII.A.1. Subject Medical Outcome Metrics

As referred to herein, the term “morbidity” with regard to a subject refers to a measure of ailment of the subject. In some embodiments, morbidity of a subject can be measured based on duration of mechanical ventilation, duration of renal replacement therapy, duration of renal failure, duration of vasopressors, incidence of ICU readmission within 48 hours of discharge, incidence of acute organ failure according to SOFA score, incidence of ICU-acquired weakness assessed using the Medical Research Council scale, subject and/or subject family satisfaction, delirium-free days at 28 days (assessed using e.g., the Confusion Assessment Method for the ICU (CAM-ICU) or Brief Confusion Assessment Method (bCAM) for the ED), coma-free days at 28 days (assessed using e.g., the Richmond Agitation-Sedation Scale (RASS)), organ failure-free days at 28 days (assessed using e.g., SOFA score or Pediatric Logistic Organ Dysfunction (PELOD-2)).

As referred to herein, the term “mortality” with regard to a subject refers to death of the subject. In some embodiments, mortality of a subject can be quantified based on period of time between diagnosis/intervention recommendation of the subject and death of the subject. For example, a subject that receives a diagnosis/intervention recommendation at day 0 and passes away at day 90 can be identified as having a 90-day mortality. Similarly, subjects can be identified as having 7-day mortality, 28-day mortality, 30-day mortality, 60-day mortality, or 1-year mortality. Thus reduced mortality of a subject can comprise an increased period of time between diagnosis/intervention recommendation of the subject and death of the subject. In alternative embodiments, mortality of a subject can be defined based on a location of death of the subject. For example, a subject that passes away in a hospital can be identified as having hospital mortality. Similarly, a subject that passes away in the ICU can be identified as having ICU mortality. In such embodiments, reduced mortality of a subject can comprise a decrease or absence of hospital and/or ICU mortality.

As referred to herein, the term “intervention” with regard to a subject refers to any medical intervention provided to the subject. For example, an intervention for a subject can include administration of vasopressors, ventilators, acute renal therapy replacement therapy (e.g., hemodialysis, continuous venovenous hemofiltration (CVVH), continuous venovenous hemodialysis (CVVHD), and peritoneal dialysis), extracorporeal membrane oxygenation (ECMO), medical device use, and/or any other form of medical intervention. Thus intervention-free days for a subject refers to a consecutive quantity of days after which the subject does not require medical intervention. For example, intervention free days can refer to a subject being vasopressor-free at 28 days, ventilator-free at 28 days, central venous line-free at 28 days, intensive care unit (ICU)-free at 28 days, dialysis (e.g., conventional hemodialysis, continuous venovenous hemofiltration (CVVH), continuous venovenous hemodialysis (CVVHD), and peritoneal dialysis)-free at 28 days, corticosteroid-free at 28 days, and/or extracorporeal membrane oxygenation (ECMO)-free at 28 days.

The cost of a stay of a subject at a patient care center at which the subject receives the medical diagnosis recommendation and/or intervention recommendation from the system can be determined by any means. For example, the cost of a stay of a subject can comprise a cost of an ICU stay of the subject, which can in turn be determined by summing at least one of a cost of the subject's admission to the ICU, stay at the ICU, primary care visits, specialty care, emergency department visits, hospital readmissions up to 90 days following ICU discharge, medical devices provided upon ICU discharge, hospital admission costs, and/or any other cost metric associated with the subject's stay at the patient care center.

Similarly, the length of a stay of a subject at a patient care center at which the subject receives the medical diagnosis recommendation and/or intervention recommendation from the system can be determined by any means. For example, the length of a stay of a subject can be determined by summing at least one of a length of primary care, specialty care, emergency department visits, ICU stay, hospital readmissions up to 90 days following discharge, and any other time metric associated with the subject's stay at the patient care center.

As referred to herein, the term “adverse event” with regard to a subject refers to any negative medical event of a subject that results in disrupted health of the subject. For example, in some embodiments, an adverse event of a subject can comprise ventilator-associated pneumonia, stroke, hemorrhagic complications, blood products (red blood cell, platelets, fresh frozen plasm) transfusion, pulmonary embolism, acute coronary syndrome, cardiac arrest, atrial fibrillation, mesenteric ischemia, life-threatening arrhythmia, pneumothorax, hyperglycemia, gastroinutilizeinal hemorrhage, delirium, hospital-acquired infection, and/or any other health affliction of the subject.

As referred to herein, the term “patient quality score” with regard to a subject refers to any measure of patient heath. For example, in some embodiments, a patient quality score for a subject can comprise a health-related quality of life (HRQL) indicator and functional indicator after ICU and hospital discharge (e.g., assessed using the Montreal Cognitive Assessment (MoCA)), a FSS-ICU score, a score from the new five-level version of the EQ-5D questionnaire (i.e., the EQ-5D-5L questionnaire), a PTSD incidence post-hospital discharge checklist score (e.g., assessed using the PTSD check list-civilian (PCL-C)), a score from a quality of well-being scale, a EuroQol score, a Nottingham Health profile, a short form 36/12 score, a sickness impact profile, a health utilities index score, a Pediatric Quality of Life Inventory (e.g., PedsQL) score, a PedsQL™ 2.0 Family Impact Module score, a Pediatric Logistic Organ Dysfunction (PELOD-2) instrument score, a quantity of delirium and coma-free days (DCFDs) of the subject, a Richmond Agitation-Sedation Scale (RASS) score, a Confusion Assessment Method for the ICU (CAM-ICU) score, a Brief Confusion Assessment Method (bCAM) score, a trichotomous mortality/morbidity outcome, a Pediatric Overall Performance Category (POPC) score, a Functional Status Scale (FSS) score, and/or any alternative score of quality of health of the subject.

As referred to herein, the term “patient care center quality score” with regard to a patient care center refers to any hospital, insurance organization, state, national, and/or other agency-specific metric for measuring the quality of care provided at a health care organization. Examples of patient care center quality metrics include the Agency for Healthcare Research and Quality (AHRQ) Patient Safety Indicator 13 (PSI-13) Postoperative Sepsis Rate, the Agency for Healthcare Research and Quality (AHRQ) Inpatient Quality Indicator 20 (IQI-20) Pneumonia Mortality rate, CMS Hospital Value Based Purchasing (VBP) Medicare Spending Per Beneficiary Rate (MSPB), and National Database of Nursing Quality Indicators (NDNQI) Nosocomial Infections Rate, and any other patient care center quality metric.

As referred to herein, the term “patient throughput” with regard to a patient care center refers to a quantity of subjects processed by the patient care center in a given period of time. For example, in one embodiment, patient throughout of a patient care center refers to a quantity of subjects intaken and/or discharged at a patient care center in a given period of time.

As referred to herein, the term “patient satisfaction” with regard to a patient care center refers to patient-reported experience with a patient care center as measured by any survey instrument. Survey instruments can include CMS-mandated and/or hospital-selected patient care questions on an official and/or unofficial HCAHPS survey answered via patient mail, telephone, email, any other medium, and/or collected in real-time by hospital staff, third parties, and/or electronic devices prior to, during, and/or after the care encounter. For example, surveys conducted by nurse hourly-rounding feedback and/or patient discharge surveys may be used as measures of patient satisfaction. Examples of a relevant survey questions measuring patient satisfaction include: “Would you recommend this hospital to friends and family” and “Using any number from 0 to 10, where 0 is the worst hospital possible and 10 is the best hospital possible, what number would you use to rate this hospital during your stay?”.

### VIII.A.2. Fundamental Predictive Diagnostic Metrics

As referred to herein, the term “sensitivity” with regard to diagnoses refers to a measure of a proportion of true positive diagnoses correctly identified. Sensitivity can be calculated as a ratio of true positive diagnoses to a sum of true positive diagnoses and false negative diagnoses. In some embodiments, sensitivity can also be referred to as “recall” or “true positive rate.”

As referred to herein, the term “specificity” with regard to diagnoses refers to a measure of a proportion of true negative diagnoses correctly identified. Specificity can be calculated as a ratio of true negative diagnoses to a sum of true negative diagnoses and false positive diagnoses.

As referred to herein, the term “negative predictive value” with regard to diagnoses refers to a measure of a proportion of negative diagnoses identified that are true negative diagnoses. Negative predictive value can be calculated as a ratio of true negative diagnoses to a sum of true negative diagnoses and false negative diagnoses.

As referred to herein, the term “positive predictive value” with regard to diagnoses refers to a measure of a proportion of positive diagnoses identified that are true positive diagnoses. Positive predictive value can be calculated as a ratio of true positive diagnoses to a sum or true positive diagnoses and false positive diagnoses. In some embodiments, positive predictive value can also be referred to as “precision.”

As referred to herein, the term “accuracy” with regard to diagnoses refers to a measure of a proportion of diagnoses correctly identified. Accuracy can be calculated as a ratio of a sum of true positive diagnoses and true negative diagnoses to a total quantity of diagnoses made.

As referred to herein, the term “ROC curve” with regard to diagnoses refers to a line graph that plots sensitivity against a false positive rate at various threshold values. The false positive rate can be calculated as a ratio of false positive diagnoses to a sum of false positive diagnoses and true negative diagnoses. An area under the ROC curve can be determined by calculating a definite integral between two points on the curve.

As referred to herein, the term “precision-recall curve” with regard to diagnoses refers to a line graph that plots positive predictive value against sensitivity at various threshold values. An area under the precision-recall curve can be determined by calculating a definite integral between two points on the curve.

### IX. Method Flow Chart

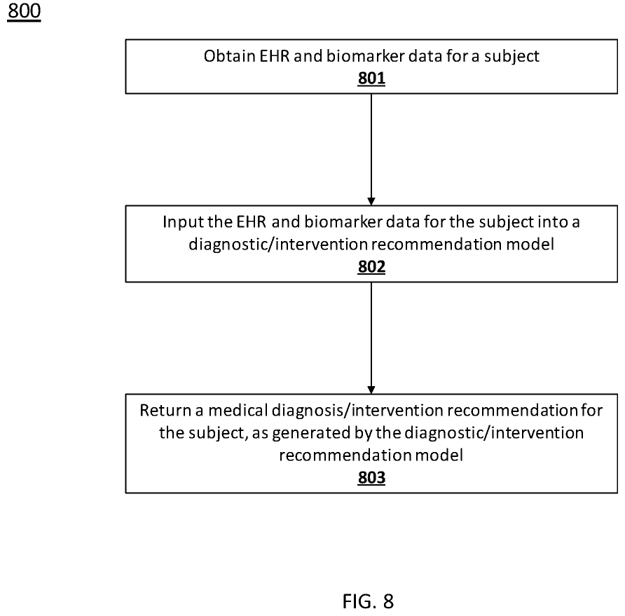

Turning next to FIG. 8, FIG. 8 is a flow chart of a method for determining a medical diagnosis/intervention recommendation for a subject, in accordance with an embodiment. In other embodiments, the method may include different and/or additional steps than those shown in FIG. 8 Additionally, steps of the method may be performed in different orders than the order described in conjunction with FIG. 8.

As shown in FIG. 8, the diagnostic/intervention recommendation system obtains 801 EHR data and biomarker data for a subject. As discussed above, EHR comprises an electronically-recorded set of medical and/or health information for a subject. EHR data can comprise any type of medical and/or health data for a subject, and can be collected by any means. Biomarker data comprises data describing the presence or absence of one or more measurable substances in a sample from a subject. Biomarker data can comprise any measurable substance from any sample from a subject, and can be determined by any means.

In some embodiments, biomarker data can be determined from a subject's sample using an in vitro diagnostic device (IVD). More specifically, in some embodiments, biomarker data for a subject can be automatically received at the diagnostic/intervention recommendation system from an IVD that identified the biomarker data for the subject from a sample from the subject. In such embodiments in which biomarker data is received from an IVD, the biomarker data can comprise at least one of genomic, epigenomic, transcriptomic, proteomic, metabolomic, and lipidomic data for the subject.

After the diagnostic/intervention recommendation system obtains 801 the EHR and biomarker data for the subject, the diagnostic/intervention recommendation system inputs 802 the EHR and biomarker data for the subject into a diagnostic/intervention recommendation model that in part comprises the diagnostic/intervention recommendation system. As discussed above, the diagnostic/intervention recommendation model is configured to receive inputs of EHR data and biomarker data for a subject and to determine a medical diagnosis/intervention recommendation for the subject. In general, the diagnostic/intervention recommendation model comprises a function modified by a plurality of parameters to accurately capture the relationship between independent variables (e.g., EHR and biomarker data) and dependent variables (e.g., diagnosis/intervention recommendation) in a training dataset.

More specifically, the plurality of parameters of the diagnostic/intervention recommendation model are identified based at least on a training dataset comprising a plurality of training samples. Each training sample of the plurality of training samples is associated with a retrospective subject, and comprises EHR data and biomarker data for the respective subject. In embodiments in which the diagnostic/intervention recommendation model is trained using supervised learning, each training sample further comprises a retrospective medical diagnosis/intervention for the retrospective subject and a retrospective medical outcome of the retrospective subject following receipt of the medical diagnosis/intervention. The function represents a relation between the EHR data and the biomarker data for the subject input into the diagnostic/intervention recommendation model, and the medical diagnosis/intervention recommendation for the subject generated as an output of the diagnostic/intervention recommendation model based on the EHR data and the biomarker data for the subject, and the plurality of parameters identified at least based on the training dataset.

In some embodiments, the diagnostic/intervention recommendation model is stored by a primary system in communication with one or more third-party systems remote from the primary system. In such embodiments, the plurality of parameters of the diagnostic/intervention recommendation model can be identified by providing the diagnostic/intervention recommendation system from the primary system to the one or more third-party systems via network transmission. As used herein, the term “network transmission” can include transmission of data via the internet, wireless transmission of data, non-wireless transmission of data (e.g., transmission of data via ethernet), or any other form of data transmission. Then, the plurality of parameters of the diagnostic/intervention recommendation model can be identified at the one or more third-party systems as discussed above, using a training set received at the one or more third-party systems.

Based on the EHR and biomarker data for the subject input into the diagnostic/intervention recommendation model, the diagnostic/intervention recommendation model generates a diagnosis/intervention recommendation for the subject. Then, the diagnostic/intervention recommendation system returns 803 the medical diagnosis/intervention recommendation for the subject, as generated by the diagnostic/intervention recommendation model. A diagnosis recommendation returned by the diagnostic/intervention recommendation system comprises an identification of a medical condition of a subject. An intervention recommendation returned by the diagnostic/intervention recommendation system comprises an identification of a medical intervention for a subject.

In some embodiments, the medical diagnosis/intervention recommendation for the subject returned by the diagnostic/intervention recommendation system fulfills at least one of the following conditions when compared to a standard-of-care medical diagnosis/intervention for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation and/or the medical intervention recommendation to the subject, reduced cost of stay of the subject at a patient care center at which the subject receives the medical diagnosis recommendation and/or the medical intervention recommendation, reduced length of stay of the subject at a patient care center at which the subject receives the medical diagnosis recommendation and/or the medical intervention recommendation, reduced quantity of adverse events of the subject, improved patient quality scores of the subject, improved patient care center quality scores for a patient care center at which the subject receives the medical diagnosis recommendation and/or the medical intervention recommendation, improved patient satisfaction with a patient care center at which the subject receives the medical diagnosis recommendation and/or the medical intervention recommendation, increased patient throughput at a patient care center at which the subject receives the medical diagnosis recommendation and/or the medical intervention recommendation, and increased revenue of a patient care center at which the subject receives the medical diagnosis recommendation and/or the medical intervention recommendation.

Finally, in certain embodiments in which the diagnostic/intervention recommendation system generates and returns medical intervention recommendations for subjects, the diagnostic/intervention recommendation system can also generate a dataset that provides evidence in support of an indication for a medical intervention. In such embodiments, the medical intervention is determined by the diagnostic/intervention recommendation model using EHR data and biomarker data for one or more subjects. The indication comprises values for at least one of the EHR data and biomarker data used by the diagnostic/intervention recommendation model to determine the medical intervention for the one or more subjects, and is based on a medical outcome of the one or more subjects.

### X. Example Computer

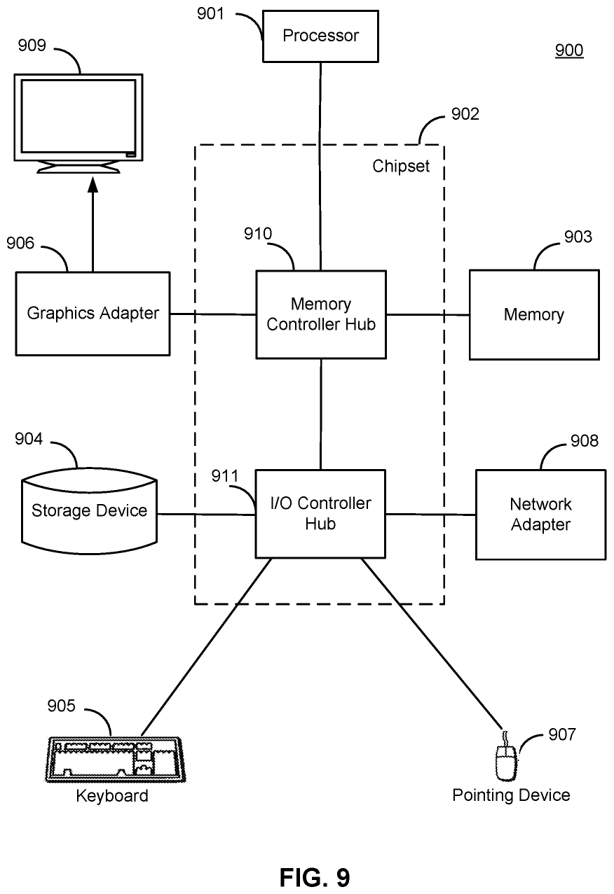

FIG. 9 illustrates an example computer 900 for implementing the method described in FIG. 8, in accordance with an embodiment. The computer 900 includes at least one processor 901 coupled to a chipset 902. The chipset 902 includes a memory controller hub 910 and an input/output (I/O) controller hub 911. A memory 903 and a graphics adapter 906 are coupled to the memory controller hub 910, and a display 909 is coupled to the graphics adapter 906. A storage device 904, an input device 907, and network adapter 908 are coupled to the I/O controller hub 911. Other embodiments of the computer 900 have different architectures.

The storage device 904 is a non-transitory computer-readable storage medium such as a hard drive, compact disk read-only memory (CD-ROM), DVD, or a solid-state memory device. The memory 903 holds instructions and data used by the processor 901. The input interface 907 is a touch-screen interface, a mouse, track ball, or other type of pointing device, a keyboard, or some combination thereof, and is used to input data into the computer 900. In some embodiments, the computer 900 can be configured to receive input (e.g., commands) from the input interface 907 via gestures from the user. The graphics adapter 906 displays images and other information on the display 909. The network adapter 908 couples the computer 900 to one or more computer networks.

The computer 900 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 904, loaded into the memory 903, and executed by the processor 901.

The types of computers 900 used to implement the method of FIG. 8 can vary depending upon the embodiment and the processing power required by the entity. For example, the diagnostic/intervention recommendation system can run in a single computer 900 or multiple computers 900 communicating with each other through a network such as in a server farm. The computers 900 can lack some of the components described above, such as graphics adapters 906, and displays 909.

### XII. Additional Embodiments

In one aspect, the invention provides a method for determining a medical intervention recommendation for a subject. The method includes obtaining electronic health record (EHR) data and biomarker data for the subject, optionally transforming the data into a common format, inputting the transformed EHR data and biomarker data for the subject into an intervention recommendation model, and returning a medical intervention recommendation for the subject output by the intervention recommendation model. The EHR data and the biomarker data for the subject are input into the intervention recommendation model using a computer processor.

In various embodiments, the intervention recommendation model comprises a plurality of parameters and a function. The function represents a relation between the EHR data and the biomarker data for the subject received as inputs to the intervention recommendation model, and the medical intervention recommendation for the subject generated as an output of the intervention recommendation model based on the EHR data and the biomarker data for the subject and the plurality of parameters. The parameters are identified prior to use of the model, during training of the model, at least based on a training dataset. The training dataset comprises a plurality of training samples. Each training sample is associated with a retrospective subject and comprises EHR data for the retrospective subject and biomarker data for the retrospective subject.

In various embodiments, the intervention recommendation model is a statistically derived model comprising a function that relates the EHR data and the biomarker data for the subject to the medical intervention recommendation. In other words, the intervention recommendation model can be a non-machine learned model. In various embodiments, the intervention recommendation model can be a machine-learned model for which the plurality of parameters comprising the model are learned by a computer based on the training dataset. The plurality of parameters, and thus the model, are learned by a computer because it would be too difficult or too inefficient for the parameters to be identified by a human based on the training dataset due to the size and/or complexity of the training dataset. In some embodiments, the intervention recommendation model can be a discretely programmed model. In alternative embodiments, the intervention recommendation model can be learned via unsupervised learning (e.g., clustering). In further embodiments, the intervention recommendation model can be learned via supervised learning. For example, the intervention recommendation model can be a classifier or a regression model.

In embodiments in which the intervention recommendation model is learned via supervised learning, the intervention recommendation model can be trained based on outcome data. Specifically, in embodiments in which the intervention recommendation model is learned via supervised learning, each training sample of the training dataset can further include a medical intervention provided to the retrospective subject associated with the training sample and a medical outcome of the retrospective subject associated with the training sample, such that the intervention recommendation model is trained to optimize the medical outcome of subjects.

EHR data for a subject comprises an electronically-recorded set of medical and/or health information for the subject. EHR data can comprise any type of medical and/or health data for a subject, and can be collected by any means. For example, EHR data can be collected and electronically recorded at a patient care center (e.g., a physician's office, the emergency department of a hospital, the intensive care unit of a hospital, the ward of a hospital), a clinical laboratory, a research laboratory, a remote consumer medical device, a therapeutic device (e.g., an infusion pump), a monitoring device such as a wearable device (e.g., a heart rate monitor), and any other site. EHR data can also be obtained from any private, public, and/or commercial source. EHR data used to train the model can be retrospective data. EHR data used to train and/or use the model can be prospective data.

Biomarker data for a subject is obtained from a sample from the subject, and comprises data describing the presence or absence of one or more measurable substances in the sample. In a preferred embodiment, biomarker data can comprise at least one of genomic data, epigenomic, transcriptomic data, proteomic data, metabolic data, and lipidomic data. In further embodiments, biomarker data can comprise a quantification of expression of each of a plurality of genes in a specified gene panel. In a preferred embodiment, a sample from a subject that is used to determine biomarker data comprises at least one of a blood sample, a urine, stool, bronchial lavage, tissue, mucus, or other bodily sample. In some embodiments, a sample from a subject that is used to determine biomarker data is collected by one or more of a FDA-cleared, commercially-available sample collection, transport, and processing device.

Biomarker data can be determined from a subject's sample using clinical laboratory equipment, an in vitro diagnostic (IVD) device, a research-use-only device, and any other means of biomarker data determination or collection. Biomarker data for a subject can be automatically received from an IVD device. In such embodiments in which the biomarker data for a subject is received from an IVD device, the biomarker data can include at least one of genomic, epigenomic, transcriptomic, proteomic, metabolomic, and lipidomic data for the subject.

In embodiments in which the biomarker data 103 comprises proteomic data, the biomarker data 103 can be determined from the subject's sample by at least one of mass spectrometry and immunoassay. In embodiments in which the biomarker data 103 comprises proteomic data, the biomarker data 103 can be determined from the subject's sample by at least one of mass spectrometry and immunoassay. In embodiments in which the biomarker data 103 comprises genomic data, the biomarker data 103 can be determined from the subject's sample by at least one of exome and whole genome nucleotide sequencing. In embodiments in which the biomarker data 103 comprises transcriptomic data, the biomarker data 103 can be determined from the subject's sample by at least one of microarray, RNA sequencing, and RT-qPCR.

A sample from a subject used to determine biomarker data can be collected at any site, and biomarker data can be determined using the collected sample at any site, prior to being input into intervention recommendation model. For example, a sample from a subject can be collected at a patient care center (e.g., a physician's office, a hospital), a clinical laboratory, a CLIA-certified laboratory, a research laboratory, a remote location, and any other site. Similarly, biomarker data can be determined using the collected sample at a patient care center (e.g., a physician's office, a hospital), a clinical laboratory, a CLIA-certified laboratory, a research laboratory, a remote location, and any other site. In certain embodiments, biomarker data for a subject is determined at the same site at which the sample from the subject was collected. For example, biomarker data can be obtained from a sample from a subject on-site at a patient care center at which the subject provided the sample. In alternative embodiments, biomarker data for a subject can be determined at a different site from which the sample from the subject was collected. For example, biomarker data can be obtained from a sample from a subject off-site from a patient care center at which the subject provided the sample. Biomarker data can also be obtained from any private, public, and/or commercial source. Biomarker data used to train the model can be retrospective data. On the other hand, biomarker data used to train and/or use the model can be prospective data.

The medical condition with which a subject is diagnosed can include, e.g., one of sepsis, septic shock, refractory septic shock, acute lung injury, acute respiratory distress syndrome, acute renal failure, acute kidney injury, trauma, burns, COVID19, pneumonia, viral infection, and post-operative conditions (e.g., conditions following open heart surgery). The medical intervention recommendation determined for a subject by the intervention recommendation model can include at least one of a selection, dosage, timing, starting, stopping, and monitoring of one or more pharmaceutical compounds, drugs, and biologics. In some cases, the determined medical intervention recommendation is a non-pharmaceutical intervention. In some cases, the non-pharmaceutical intervention determined for a subject is the collection of a biospecimen from the subject and/or the collection of electronic health record data from the subject.

The medical intervention recommendation for the subject output by the intervention recommendation model can fulfill at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, improved patient satisfaction with a patient care center at which the subject receives the medical intervention recommendation, increased patient throughput at a patient care center at which the subject receives the medical intervention recommendation, and increased revenue of a patient care center at which the subject receives the medical intervention recommendation.

In some embodiments, the method can further comprise generating a dataset that provides evidence in support of an indication for a medical intervention for a condition. The medical intervention is determined by the intervention recommendation model using EHR data and biomarker data for one or more subjects diagnosed with the condition as discussed above. The indication comprises values for at least one of EHR data and biomarker data used by the intervention recommendation model to determine the medical intervention for one or more subjects, and is based on a medical outcome of the one or more subjects.

In some embodiments, the intervention recommendation model is stored by a primary system that is in communication with one or more third-party systems. The one or more third-party systems can be remote from the primary system. The one or more third-party systems can also be located at one or more patient care centers. In such embodiments, the intervention recommendation model can be alternatively trained and utilized between the primary system and the one or more third-party systems.

For example, in a first embodiment, the intervention recommendation model can be both trained and utilized at the primary system. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, EHR data and biomarker data obtained for the subject are received from the one or more third-party systems at the primary system. Then, the medical intervention recommendation generated for the subject by the intervention recommendation model is generated at the primary system using the EHR data and the biomarker data for the subject.

In an alternative embodiment, the intervention recommendation model can be trained at the primary system, but utilized at the one or more third-party systems. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, the trained model is provided to the one or more third-party systems via network transmission. In some cases, the trained model is automatically provided to the third-party systems at specified time intervals or in real-time or near real-time following identification of the model parameters. The EHR data and biomarker data obtained for the subject are received at the model at the third-party systems, and finally, the medical intervention recommendation generated for the subject by the intervention recommendation model is generated at the third-party systems using the EHR data and the biomarker data for the subject.

In a third embodiment, the intervention recommendation model can conversely be trained at the one or more third-party systems, but utilized at the primary system. In such an embodiment, the intervention recommendation model is provided to the one or more third-party systems from the primary system via network transmission. Then, the intervention recommendation model receives one or more of the training samples of the training dataset at the third-party systems. At the third-party systems, the plurality of parameters of the model are identified using the received training samples. To utilize the model, the trained model is received at the primary system via network transmission. In some cases, the trained model is automatically provided to the primary system at specified time intervals or in real-time or near real-time following identification of the model parameters. Then, EHR data and biomarker data for the subject are received from the one or more third-party systems at the primary system, and the medical intervention recommendation for the subject is generated by the model at the primary system using the EHR data and the biomarker data received for the subject.

Finally, in an alternative embodiment, the intervention recommendation model can be both trained and utilized at the one or more third-party systems. In such an embodiment, the intervention recommendation model is provided to the one or more third-party systems from the primary system via network transmission. Then, the intervention recommendation model receives one or more training samples of the training dataset at the third-party systems. At the third-party systems, the plurality of parameters of the model are identified using the training samples. To utilize the model, the EHR data and the biomarker data obtained for the subject are received by the model at the third-party systems, and the medical intervention recommendation generated for the subject by the intervention recommendation model is generated at the third-party systems using the EHR data and the biomarker data for the subject.

In cases in which the intervention recommendation model is trained at the primary system, the plurality of training samples can be received at the primary system via network transmission from the one or more third-party systems. In some instances, one or more of the plurality of training samples can be received from multiple distinct third-party systems and can comprise different data formats. In such cases, the training samples can be transformed into a common data format, and the transformed training samples can be merged into a merged training dataset. This merged training dataset can then be used to identify the model parameters as discussed above.

In alternative embodiments in which the intervention recommendation model is trained at the one or more third-party systems rather than the primary system, the training samples can be received at multiple, distinct third-party systems.

Regardless of where the model is trained, the plurality of training samples can be automatically received at specified time intervals such that the parameters of the model are automatically identified at specified time intervals and such that the model is automatically updated at specified time intervals. Alternatively, the plurality of training samples can be automatically received in real-time or near real-time such that the parameters of the model are automatically identified in real-time or near real-time and such that the model is automatically updated in real-time or near real-time.

When the model is utilized at the primary system, the EHR data and biomarker data for the subject can be received at the primary system via network transmission from the one or more third-party systems. Furthermore, the medical intervention recommendation for the subject output by the intervention recommendation model can be provided by the primary system to the one or more third-party systems via network transmission.

Alternatively, when the intervention recommendation model is utilized at the one or more third-party systems, the medical intervention recommendation for the subject output by the intervention recommendation model can be provided to the subject.

In another aspect, the invention provides a non-transitory computer-readable storage medium that stores computer program instructions that, when executed by a computer processor, cause the computer processor to determine a medical intervention recommendation for the subject by performing any combination of the above method steps.

In yet another aspect, the invention provides a method for determining a medical diagnosis recommendation for a subject. The method includes obtaining electronic health record (EHR) data and biomarker data for the subject, optionally transforming the data into a common format, inputting the transformed EHR data and biomarker data for the subject into a diagnostic recommendation model, and returning a medical diagnosis recommendation for the subject output by the diagnostic recommendation model. The EHR data and the biomarker data for the subject are input into the diagnostic recommendation model using a computer processor.

In various embodiments, the diagnostic recommendation model comprises a plurality of parameters and a function. The function represents a relation between the EHR data and the biomarker data for the subject received as inputs to the diagnostic recommendation model, and the medical diagnosis recommendation for the subject generated as an output of the diagnostic recommendation model based on the EHR data and the biomarker data for the subject and the plurality of parameters. The parameters are identified prior to use of the model, during training of the model, at least based on a training dataset. The training dataset comprises a plurality of training samples. Each training sample is associated with a retrospective subject and comprises EHR data for the retrospective subject and biomarker data for the retrospective subject.

In various embodiments, the diagnostic recommendation model is a statistically derived model comprising a function that relates the EHR data and the biomarker data for the subject to the medical intervention recommendation. In other words, the diagnostic recommendation model is a non-machine learned model. In various embodiments, the diagnostic recommendation model is a machine-learned model. The diagnostic recommendation model can be any model for which the plurality of parameters comprising the model are learned by a computer based on the training dataset. The plurality of parameters, and thus the model, are learned by a computer because it would be too difficult or too inefficient for the parameters to be identified by a human based on the training dataset due to the size and/or complexity of the training dataset. In some embodiments, the diagnostic recommendation model can be a discretely programmed model. In alternative embodiments, the diagnostic recommendation model can be learned via unsupervised learning (e.g., clustering). In further embodiments, the diagnostic recommendation model can be learned via supervised learning. For example, the diagnostic recommendation model can be a classifier or a regression model.

In embodiments in which the diagnostic recommendation model is learned via supervised learning, the diagnostic recommendation model can be trained based on outcome data. Specifically, in embodiments in which the diagnostic recommendation model is learned via supervised learning, each training sample of the training dataset can further include a medical diagnosis provided to the retrospective subject associated with the training sample and a medical outcome of the retrospective subject associated with the training sample, such that the diagnostic recommendation model is trained to optimize the medical outcome of subjects.

EHR data for a subject comprises an electronically-recorded set of medical and/or health information for the subject. EHR data can comprise any type of medical and/or health data for a subject, and can be collected by any means. For example, EHR data can be collected and electronically recorded at a patient care center (e.g., a physician's office, the emergency department of a hospital, the intensive care unit of a hospital, the ward of a hospital), a clinical laboratory, a research laboratory, a remote consumer medical device, a therapeutic device (e.g., an infusion pump), a monitoring device such as a wearable device (e.g., a heart rate monitor), and any other site. EHR data can also be obtained from any private, public, and/or commercial source. EHR data used to train the model can be retrospective data. EHR data used to train and/or use the model can be prospective data.

Biomarker data for a subject is obtained from a sample from the subject, and comprises data describing the presence or absence of one or more measurable substances in the sample. In a preferred embodiment, biomarker data can comprise at least one of genomic data, epigenomic data, transcriptomic data, proteomic data, metabolic data, and lipidomic data. In further embodiments, biomarker data can comprise a quantification of expression of each of a plurality of genes in a specified gene panel. In a preferred embodiment, a sample from a subject that is used to determine biomarker data comprises at least one of a blood sample, a urine, stool, bronchial lavage, tissue, mucus, or other bodily sample. In some embodiments, a sample from a subject that is used to determine biomarker data is collected by one or more of a FDA-cleared, commercially-available sample collection, transport, and processing device.

Biomarker data can be determined from a subject's sample using clinical laboratory equipment, an in vitro diagnostic (IVD) device, a research-use-only device, and any other means of biomarker data determination or collection. Biomarker data for a subject can be automatically received from an IVD device. In such embodiments in which the biomarker data for a subject is received from an IVD device, the biomarker data can include at least one of genomic, epigenomic, transcriptomic, proteomic, metabolomic, and lipidomic data for the subject.

In embodiments in which the biomarker data 103 comprises proteomic data, the biomarker data 103 can be determined from the subject's sample by at least one of mass spectrometry and immunoassay. In embodiments in which the biomarker data 103 comprises genomic data, the biomarker data 103 can be determined from the subject's sample by at least one of exome and whole genome nucleotide sequencing. In embodiments in which the biomarker data 103 comprises transcriptomic data, the biomarker data 103 can be determined from the subject's sample by at least one of microarray, RNA sequencing, and RT-qPCR.

A sample from a subject used to determine biomarker data can be collected at any site, and biomarker data can be determined using the collected sample at any site, prior to being input into diagnostic recommendation model. For example, a sample from a subject can be collected at a patient care center (e.g., a physician's office, a hospital), a clinical laboratory, a CLIA-certified laboratory, a research laboratory, a remote location, and any other site. Similarly, biomarker data can be determined using the collected sample at a patient care center (e.g., a physician's office, a hospital), a clinical laboratory, a CLIA-certified laboratory, a research laboratory, a remote location, and any other site. In certain embodiments, biomarker data for a subject is determined at the same site at which the sample from the subject was collected. For example, biomarker data can be obtained from a sample from a subject on-site at a patient care center at which the subject provided the sample. In alternative embodiments, biomarker data for a subject can be determined at a different site from which the sample from the subject was collected. For example, biomarker data can be obtained from a sample from a subject off-site from a patient care center at which the subject provided the sample. Biomarker data can also be obtained from any private, public, and/or commercial source. Biomarker data used to train the model can be retrospective data. On the other hand, biomarker data used to utilize the model can be prospective data.

The medical diagnosis recommendation for the subject output by the diagnostic recommendation model can include one of sepsis, septic shock, refractory septic shock, acute lung injury, acute respiratory distress syndrome, acute renal failure, acute kidney injury, trauma, burns, COVID19, pneumonia, viral infection, and post-operative conditions (e.g., conditions following open heart surgery). In some embodiments, the method further includes a step of providing a medical intervention to the subject based on the determined medical diagnosis recommendation. In such embodiments, the medical intervention can include at least one of a selection, dosage, timing, starting, stopping, and monitoring of one or more pharmaceutical compounds, drugs, and biologics. In alternative embodiments, the medical intervention can be a non-pharmaceutical intervention. In some cases, the non-pharmaceutical intervention determined for a subject is the collection of a biospecimen from the subject and/or the collection of electronic health record data from the subject.

The medical diagnosis recommendation output by the diagnostic recommendation model can fulfill at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, improved patient satisfaction with a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

In some embodiments, the diagnostic recommendation model is stored by a primary system that is in communication with one or more third-party systems. The one or more third-party systems can be remote from the primary system. The one or more third-party systems can also be located at one or more patient care centers. In such embodiments, the diagnostic recommendation model can be alternatively trained and utilized between the primary system and the one or more third-party systems.

For example, in a first embodiment, the diagnostic recommendation model can be both trained and utilized at the primary system. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, EHR data and biomarker data obtained for the subject are received from the one or more third-party systems at the primary system. Then, the medical diagnosis recommendation generated for the subject by the diagnostic recommendation model is generated at the primary system using the EHR data and the biomarker data for the subject.

In an alternative embodiment, the diagnostic recommendation model can be trained at the primary system, but utilized at the one or more third-party systems. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, the trained model is provided to the one or more third-party systems via network transmission. In some cases, the trained model is automatically provided to the third-party systems at specified time intervals or in real-time following identification of the model parameters. The EHR data and biomarker data obtained for the subject are received at the model at the third-party systems, and finally, the medical diagnosis recommendation generated for the subject by the diagnostic recommendation model is generated at the third-party systems using the EHR data and the biomarker data for the subject.

In a third embodiment, the diagnostic recommendation model can conversely be trained at the one or more third-party systems, but utilized at the primary system. In such an embodiment, the diagnostic recommendation model is provided to the one or more third-party systems from the primary system via network transmission. Then, the diagnostic recommendation model receives one or more of the training samples of the training dataset at the third-party systems. At the third-party systems, the plurality of parameters of the model are identified using the received training samples. To utilize the model, the trained model is received at the primary system via network transmission. In some cases, the trained model is automatically provided to the primary system at specified time intervals or in real-time or near real-time following identification of the model parameters. Then, EHR data and biomarker data for the subject are received from the one or more third-party systems at the primary system, and the medical diagnosis recommendation for the subject is generated by the model at the primary system using the EHR data and the biomarker data received for the subject.

Finally, in an alternative embodiment, the diagnostic recommendation model can be both trained and utilized at the one or more third-party systems. In such an embodiment, the diagnostic recommendation model is provided to the one or more third-party systems from the primary system via network transmission. Then, the diagnostic recommendation model receives one or more training samples of the training dataset at the third-party systems. At the third-party systems, the plurality of parameters of the model are identified using the training samples. To utilize the model, the EHR data and the biomarker data obtained for the subject are received by the model at the third-party systems, and the medical diagnosis recommendation generated for the subject by the diagnostic recommendation model is generated at the third-party systems using the EHR data and the biomarker data for the subject.

In cases in which the diagnostic recommendation model is trained at the primary system, the plurality of training samples can be received at the primary system via network transmission from the one or more third-party systems. In some instances, one or more of the plurality of training samples can be received from multiple distinct third-party systems and can comprise different data formats. In such cases, the training samples can be transformed into a common data format, and the transformed training samples can be merged into a merged training dataset. This merged training dataset can then be used to identify the model parameters as discussed above.

In alternative embodiments in which the diagnostic recommendation model is trained at the one or more third-party systems rather than the primary system, the training samples can be received at multiple, distinct third-party systems.

Regardless of where the model is trained, the plurality of training samples can be automatically received at specified time intervals such that the parameters of the model are automatically identified at specified time intervals and such that the model is automatically updated at specified time intervals. Alternatively, the plurality of training samples can be automatically received in real-time such that the parameters of the model are automatically identified in real-time and such that the model is automatically updated in real-time.

When the model is utilized at the primary system, the EHR data and biomarker data for the subject can be received at the primary system via network transmission from the one or more third-party systems. Furthermore, the medical diagnosis recommendation for the subject output by the diagnostic recommendation model can be provided by the primary system to the one or more third-party systems via network transmission.

In another aspect, the invention provides a non-transitory computer-readable storage medium that stores computer program instructions that, when executed by a computer processor, cause the computer processor to determine a medical diagnosis recommendation for the subject by performing any combination of the above method steps.

In another aspect, the invention provides a method for determining a medical intervention recommendation for a subject. The method includes obtaining electronic health record (EHR) data for the subject, transforming the data into a common format, inputting the transformed EHR data for the subject into an intervention recommendation model, and returning a recommended medical intervention for the subject output by the intervention recommendation model. The EHR data for the subject is input into the intervention recommendation model using a computer processor.

The intervention recommendation model comprises a plurality of parameters and a function. The function represents a relation between the EHR data for the subject received as an input to the intervention recommendation model, and the medical intervention recommendation for the subject generated as an output of the intervention recommendation model based on the EHR data for the subject and the plurality of parameters. The parameters are identified prior to use of the model, during training of the model, at least based on a training dataset. The training dataset comprises a plurality of training samples. Each training sample is associated with a retrospective subject and comprises EHR data for the retrospective subject.

The intervention recommendation model is any model for which the plurality of parameters comprising the model are learned by a computer based on the training dataset. The plurality of parameters, and thus the model, are learned by a computer because it would be too difficult or too inefficient for the parameters to be identified by a human based on the training dataset due to the size and/or complexity of the training dataset. In some embodiments, the intervention recommendation model can be a discretely programmed model. In alternative embodiments, the intervention recommendation model can be learned via unsupervised learning (e.g., clustering). In further embodiments, the intervention recommendation model can be learned via supervised learning. For example, the intervention recommendation model can be a classifier or a regression model.

In embodiments in which the intervention recommendation model is learned via supervised learning, the intervention recommendation model can be trained based on outcome data. Specifically, in embodiments in which the intervention recommendation model is learned via supervised learning, each training sample of the training dataset can further include a medical intervention provided to the retrospective subject associated with the training sample and a medical outcome of the retrospective subject associated with the training sample, such that the intervention recommendation model is trained to optimize the medical outcome of subjects.

EHR data for a subject comprises an electronically-recorded set of medical and/or health information for the subject. EHR data can comprise any type of medical and/or health data for a subject, and can be collected by any means. For example, EHR data can be collected and electronically recorded at a patient care center (e.g., a physician's office, the emergency department of a hospital, the intensive care unit of a hospital, the ward of a hospital), a clinical laboratory, a research laboratory, a remote consumer medical device, a therapeutic device (e.g., an infusion pump), a monitoring device such as a wearable device (e.g., a heart rate monitor), and any other site. EHR data can also be obtained from any private, public, and/or commercial source. EHR data used to train the model can be retrospective data. EHR data used to train and/or use the model can be prospective data.

The medical condition with which a subject is diagnosed can include one of sepsis, septic shock, refractory septic shock, acute lung injury, acute respiratory distress syndrome, acute renal failure, acute kidney injury, trauma, burns, COVID19, pneumonia, viral infection and post-operative conditions (e.g., conditions following open heart surgery). The medical intervention recommendation determined for a subject by the intervention recommendation model can include at least one of a selection, dosage, timing, starting, stopping, and monitoring of one or more pharmaceutical compounds, drugs, and biologics. In some cases, the determined medical intervention recommendation is a non-pharmaceutical intervention. In some cases, the non-pharmaceutical intervention recommendation determined for a subject is the collection of a biospecimen from the subject and/or the collection of electronic health record data from the subject.

The medical intervention recommendation for the subject output by the intervention recommendation model can fulfill at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, improved patient satisfaction with a patient care center at which the subject receives the medical intervention recommendation, increased patient throughput at a patient care center at which the subject receives the medical intervention recommendation, and increased revenue of a patient care center at which the subject receives the medical intervention recommendation.

In some embodiments, the method can further comprise generating a dataset that provides evidence in support of an indication for a medical intervention for a condition. The medical intervention is determined by the intervention recommendation model using EHR data for one or more subjects diagnosed with the condition as discussed above. The indication comprises values for EHR data used by the intervention recommendation model to determine the medical intervention for one or more subjects, and is based on a medical outcome of the one or more subjects.

In some embodiments, the intervention recommendation model is stored by a primary system that is in communication with one or more third-party systems. The one or more third-party systems can be remote from the primary system. The one or more third-party systems can also be located at one or more patient care centers. In such embodiments, the intervention recommendation model can be alternatively trained and utilized between the primary system and the one or more third-party systems.

For example, in a first embodiment, the intervention recommendation model can be both trained and utilized at the primary system. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, EHR data obtained for the subject is received from the one or more third-party systems at the primary system. Then, the medical intervention recommendation generated for the subject by the intervention recommendation model is generated at the primary system using the EHR data for the subject.

In an alternative embodiment, the intervention recommendation model can be trained at the primary system, but utilized at the one or more third-party systems. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, the trained model is provided to the one or more third-party systems via network transmission. In some cases, the trained model is automatically provided to the third-party systems at specified time intervals or in real-time following identification of the model parameters. The EHR data obtained for the subject is received at the model at the third-party systems, and finally, the medical intervention recommendation generated for the subject by the intervention recommendation model is generated at the third-party systems using the EHR data for the subject.

In a third embodiment, the intervention recommendation model can conversely be trained at the one or more third-party systems, but utilized at the primary system. In such an embodiment, the intervention recommendation model is provided to the one or more third-party systems from the primary system via network transmission. Then, the intervention recommendation model receives one or more of the training samples of the training dataset at the third-party systems. At the third-party systems, the plurality of parameters of the model are identified using the received training samples. To utilize the model, the trained model is received at the primary system via network transmission. In some cases, the trained model is automatically provided to the primary system at specified time intervals or in real-time following identification of the model parameters. Then, EHR data for the subject is received from the one or more third-party systems at the primary system, and the medical intervention recommendation for the subject is generated by the model at the primary system using the EHR data received for the subject.

Finally, in an alternative embodiment, the intervention recommendation model can be both trained and utilized at the one or more third-party systems. In such an embodiment, the intervention recommendation model is provided to the one or more third-party systems from the primary system via network transmission. Then, the intervention recommendation model receives one or more training samples of the training dataset at the third-party systems. At the third-party systems, the plurality of parameters of the model are identified using the training samples. To utilize the model, the EHR data obtained for the subject is received by the model at the third-party systems, and the medical intervention recommendation generated for the subject by the intervention recommendation model is generated at the third-party systems using the EHR data for the subject.

In cases in which the intervention recommendation model is trained at the primary system, the plurality of training samples can be received at the primary system via network transmission from the one or more third-party systems. In some instances, one or more of the plurality of training samples can be received from multiple distinct third-party systems and can comprise different data formats. In such cases, the training samples can be transformed into a common data format, and the transformed training samples can be merged into a merged training dataset. This merged training dataset can then be used to identify the model parameters as discussed above.

In alternative embodiments in which the intervention recommendation model is trained at the one or more third-party systems rather than the primary system, the training samples can be received at multiple, distinct third-party systems.

Regardless of where the model is trained, the plurality of training samples can be automatically received at specified time intervals such that the parameters of the model are automatically identified at specified time intervals and such that the model is automatically updated at specified time intervals. Alternatively, the plurality of training samples can be automatically received in real-time such that the parameters of the model are automatically identified in real-time and such that the model is automatically updated in real-time.

When the model is utilized at the primary system, the EHR data for the subject can be received at the primary system via network transmission from the one or more third-party systems. Furthermore, the medical intervention recommendation for the subject output by the intervention recommendation model can be provided by the primary system to the one or more third-party systems via network transmission.

Alternatively, when the intervention recommendation model is utilized at the one or more third-party systems, the medical intervention recommendation for the subject output by the intervention recommendation model can be provided to the subject.

In another aspect, the invention provides a non-transitory computer-readable storage medium that stores computer program instructions that, when executed by a computer processor, cause the computer processor to determine a medical intervention recommendation for the subject by performing any combination of the above method steps.

In yet another aspect, the invention provides a method for determining a medical diagnosis recommendation for a subject. The method includes obtaining electronic health record (EHR) data for the subject, transforming the data into a common format, inputting the transformed EHR data for the subject into a diagnostic recommendation model, and returning a recommended medical diagnosis recommendation for the subject output by the diagnostic recommendation model. The EHR data for the subject is input into the diagnostic recommendation model using a computer processor.

The diagnostic recommendation model comprises a plurality of parameters and a function. The function represents a relation between the EHR data for the subject received as an input to the diagnostic recommendation model, and the medical diagnosis recommendation for the subject generated as an output of the diagnostic recommendation model based on the EHR data for the subject and the plurality of parameters. The parameters are identified prior to use of the model, during training of the model, at least based on a training dataset. The training dataset comprises a plurality of training samples. Each training sample is associated with a retrospective subject and comprises EHR data for the retrospective subject.

The diagnostic recommendation model is any model for which the plurality of parameters comprising the model are learned by a computer based on the training dataset. The plurality of parameters, and thus the model, are learned by a computer because it would be too difficult or too inefficient for the parameters to be identified by a human based on the training dataset due to the size and/or complexity of the training dataset. In some embodiments, the diagnostic recommendation model can be a discretely programmed model. In alternative embodiments, the diagnostic recommendation model can be learned via unsupervised learning (e.g., clustering). In further embodiments, the diagnostic recommendation model can be learned via supervised learning. For example, the diagnostic recommendation model can be a classifier or a regression model.

In embodiments in which the diagnostic recommendation model is learned via supervised learning, the diagnostic recommendation model can be trained based on outcome data. Specifically, in embodiments in which the diagnostic recommendation model is learned via supervised learning, each training sample of the training dataset can further include a medical diagnosis provided to the retrospective subject associated with the training sample and a medical outcome of the retrospective subject associated with the training sample, such that the diagnostic recommendation model is trained to optimize the medical outcome of subjects.

EHR data for a subject comprises an electronically-recorded set of medical and/or health information for the subject. EHR data can comprise any type of medical and/or health data for a subject, and can be collected by any means. For example, EHR data can be collected and electronically recorded at a patient care center (e.g., a physician's office, the emergency department of a hospital, the intensive care unit of a hospital, the ward of a hospital), a clinical laboratory, a research laboratory, a remote consumer medical device, a therapeutic device (e.g., an infusion pump), a monitoring device such as a wearable device (e.g., a heart rate monitor), and any other site. EHR data used to train the model can be retrospective data. EHR data used to train and/or use the model can be prospective data.

The medical diagnosis recommendation for the subject output by the diagnostic recommendation model can include one of sepsis, septic shock, refractory septic shock, acute lung injury, acute respiratory distress syndrome, acute renal failure, acute kidney injury, trauma, burns, COVID19, pneumonia, viral infection, and post-operative conditions (e.g., conditions following open heart surgery). In some embodiments, the method further includes a step of providing a medical intervention to the subject based on the determined medical diagnosis recommendation. In such embodiments, the medical intervention can include at least one of a selection, dosage, timing, starting, stopping, and monitoring of one or more pharmaceutical compounds, drugs, and biologics. In alternative embodiments, the medical intervention can be a non-pharmaceutical intervention. In some cases, the non-pharmaceutical intervention is the collection of a biospecimen from the subject and/or the collection of electronic health record data from the subject.

The medical diagnosis recommendation output by the diagnostic recommendation model can fulfill at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, improved patient satisfaction with a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

In some embodiments, the diagnostic recommendation model is stored by a primary system that is in communication with one or more third-party systems. The one or more third-party systems can be remote from the primary system. The one or more third-party systems can also be located at one or more patient care centers. In such embodiments, the diagnostic recommendation model can be alternatively trained and utilized between the primary system and the one or more third-party systems.

For example, in a first embodiment, the diagnostic recommendation model can be both trained and utilized at the primary system. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, EHR data obtained for the subject is received from the one or more third-party systems at the primary system. Then, the medical diagnosis recommendation generated for the subject by the diagnostic recommendation model is generated at the primary system using the EHR data for the subject.

In an alternative embodiment, the diagnostic recommendation model can be trained at the primary system, but utilized at the one or more third-party systems. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, the trained model is provided to the one or more third-party systems via network transmission. In some cases, the trained model is automatically provided to the third-party systems at specified time intervals or in real-time following identification of the model parameters. The EHR data obtained for the subject is received at the model at the third-party systems, and finally, the medical diagnosis recommendation generated for the subject by the diagnostic recommendation model is generated at the third-party systems using the EHR data for the subject.

In a third embodiment, the diagnostic recommendation model can conversely be trained at the one or more third-party systems, but utilized at the primary system. In such an embodiment, the diagnostic recommendation model is provided to the one or more third-party systems from the primary system via network transmission. Then, the diagnostic recommendation model receives one or more of the training samples of the training dataset at the third-party systems. At the third-party systems, the plurality of parameters of the model are identified using the received training samples. To utilize the model, the trained model is received at the primary system via network transmission. In some cases, the trained model is automatically provided to the primary system at specified time intervals or in real-time following identification of the model parameters. Then, EHR data for the subject is received from the one or more third-party systems at the primary system, and the medical diagnosis recommendation for the subject is generated by the model at the primary system using the EHR data received for the subject.

Finally, in an alternative embodiment, the diagnostic recommendation model can be both trained and utilized at the one or more third-party systems. In such an embodiment, the diagnostic recommendation model is provided to the one or more third-party systems from the primary system via network transmission. Then, the diagnostic recommendation model receives one or more training samples of the training dataset at the third-party systems. At the third-party systems, the plurality of parameters of the model are identified using the training samples. To utilize the model, the EHR data obtained for the subject is received by the model at the third-party systems, and the medical diagnosis recommendation generated for the subject by the diagnostic recommendation model is generated at the third-party systems using the EHR data for the subject.

In cases in which the diagnostic recommendation model is trained at the primary system, the plurality of training samples can be received at the primary system via network transmission from the one or more third-party systems. In some instances, one or more of the plurality of training samples can be received from multiple distinct third-party systems and can comprise different data formats. In such cases, the training samples can be transformed into a common data format, and the transformed training samples can be merged into a merged training dataset. This merged training dataset can then be used to identify the model parameters as discussed above.

In alternative embodiments in which the diagnostic recommendation model is trained at the one or more third-party systems rather than the primary system, the training samples can be received at multiple, distinct third-party systems.

Regardless of where the model is trained, the plurality of training samples can be automatically received at specified time intervals such that the parameters of the model are automatically identified at specified time intervals and such that the model is automatically updated at specified time intervals. Alternatively, the plurality of training samples can be automatically received in real-time such that the parameters of the model are automatically identified in real-time and such that the model is automatically updated in real-time.

When the model is utilized at the primary system, the EHR data for the subject can be received at the primary system via network transmission from the one or more third-party systems. Furthermore, the medical diagnosis recommendation for the subject output by the diagnostic recommendation model can be provided by the primary system to the one or more third-party systems via network transmission.

In another aspect, the invention provides a non-transitory computer-readable storage medium that stores computer program instructions that, when executed by a computer processor, cause the computer processor to determine a medical diagnosis recommendation for the subject by performing any combination of the above method steps.

In another aspect, the invention provides a method for determining a medical intervention recommendation for a subject. The method includes obtaining biomarker data for the subject, inputting the biomarker data for the subject into an intervention recommendation model, and returning the medical intervention recommendation for the subject output by the intervention recommendation model. The biomarker data for the subject is input into the intervention recommendation model using a computer processor.

The intervention recommendation model comprises a plurality of parameters and a function. The function represents a relation between the biomarker data for the subject received as an input to the intervention recommendation model, and the medical intervention recommendation for the subject generated as an output of the intervention recommendation model based on the biomarker data for the subject and the plurality of parameters. The parameters are identified prior to use of the model, during training of the model, at least based on a training dataset. The training dataset comprises a plurality of training samples. Each training sample is associated with a retrospective subject and comprises biomarker data for the retrospective subject.

The intervention recommendation model is any model for which the plurality of parameters comprising the model are learned by a computer based on the training dataset. The plurality of parameters, and thus the model, are learned by a computer because it would be too difficult or too inefficient for the parameters to be identified by a human based on the training dataset due to the size and/or complexity of the training dataset. In some embodiments, the intervention recommendation model can be a discretely programmed model. In alternative embodiments, the intervention recommendation model can be learned via unsupervised learning (e.g., clustering). In further embodiments, the intervention recommendation model can be learned via supervised learning. For example, the intervention recommendation model can be a classifier or a regression model.

In embodiments in which the intervention recommendation model is learned via supervised learning, the intervention recommendation model can be trained based on outcome data. Specifically, in embodiments in which the intervention recommendation model is learned via supervised learning, each training sample of the training dataset can further include a medical intervention provided to the retrospective subject associated with the training sample and a medical outcome of the retrospective subject associated with the training sample, such that the intervention recommendation model is trained to optimize the medical outcome of subjects.

Biomarker data for a subject is obtained from a sample from the subject, and comprises data describing the presence or absence of one or more measurable substances in the sample. In a preferred embodiment, biomarker data can comprise at least one of genomic data, epigenomic data, transcriptomic data, proteomic data, metabolic data, and lipidomic data. In further embodiments, biomarker data can comprise a quantification of expression of each of a plurality of genes in a specified gene panel. In a preferred embodiment, a sample from a subject that is used to determine biomarker data comprises at least one of a blood sample, a urine, stool, bronchial lavage, tissue, mucus, or other bodily sample. In some embodiments, a sample from a subject that is used to determine biomarker data is collected by one or more of a FDA-cleared, commercially-available sample collection, transport, and processing device.

Biomarker data can be determined from a subject's sample using clinical laboratory equipment, an in vitro diagnostic (IVD) device, a research-use-only device, and any other means of biomarker data determination or collection. Biomarker data for a subject can be automatically received from an IVD device. In such embodiments in which the biomarker data for a subject is received from an IVD device, the biomarker data can include at least one of genomic, epigenomic, transcriptomic, proteomic, metabolomic, and lipidomic data for the subject.

In embodiments in which the biomarker data 103 comprises proteomic data, the biomarker data 103 can be determined from the subject's sample by at least one of mass spectrometry and immunoassay. In embodiments in which the biomarker data 103 comprises genomic data, the biomarker data 103 can be determined from the subject's sample by at least one of exome and whole genome nucleotide sequencing. In embodiments in which the biomarker data 103 comprises transcriptomic data, the biomarker data 103 can be determined from the subject's sample by at least one of microarray, RNA sequencing, and RT-qPCR.

A sample from a subject used to determine biomarker data can be collected at any site, and biomarker data can be determined using the collected sample at any site, prior to being input into intervention recommendation model. For example, a sample from a subject can be collected at a patient care center (e.g., a physician's office, a hospital), a clinical laboratory, a CLIA-certified laboratory, a research laboratory, a remote location, and any other site. Similarly, biomarker data can be determined using the collected sample at a patient care center (e.g., a physician's office, a hospital), a clinical laboratory, a CLIA-certified laboratory, a research laboratory, a remote location, and any other site. In certain embodiments, biomarker data for a subject is determined at the same site at which the sample from the subject was collected. For example, biomarker data can be obtained from a sample from a subject on-site at a patient care center at which the subject provided the sample. In alternative embodiments, biomarker data for a subject can be determined at a different site from which the sample from the subject was collected. For example, biomarker data can be obtained from a sample from a subject off-site from a patient care center at which the subject provided the sample. Biomarker data can also be obtained from any private, public, and/or commercial source. Biomarker data used to train the model can be retrospective data. On the other hand, biomarker data used to utilize the model can be prospective data.

The medical condition with which a subject is diagnosed can include one of sepsis, septic shock, refractory septic shock, acute lung injury, acute respiratory distress syndrome, acute renal failure, acute kidney injury, trauma, burns, COVID19, pneumonia, viral infection, and post-operative conditions (e.g., conditions following open heart surgery). The medical intervention recommendation determined for a subject by the intervention recommendation model can include at least one of a selection, dosage, timing, starting, stopping, and monitoring of one or more pharmaceutical compounds, drugs, and biologics. In some cases, the determined medical intervention recommendation is a non-pharmaceutical intervention. In some cases, the non-pharmaceutical intervention recommendation determined for a subject is the collection of a biospecimen from the subject and/or the collection of electronic health record data from the subject.

The medical intervention recommendation for the subject output by the intervention recommendation model can fulfill at least one of the following conditions when compared to a standard-of-care medical intervention for a retrospective subject having at least one of the electronic health record data and the biomarker data of the subject: reduced morbidity of the subject, reduced mortality of the subject, increased quantity of intervention-free days of the subject, reduced time to provide the medical intervention recommendation to the subject, reduced cost of stay of the subject at a patient care center at which the subject receives the medical intervention recommendation, reduced length of stay of the subject at a patient care center at which the subject receives the medical intervention recommendation, reduced quantity of adverse events of the subject, improved patient quality scores of the subject, improved patient care center quality scores for a patient care center at which the subject receives the medical intervention recommendation, improved patient satisfaction with a patient care center at which the subject receives the medical intervention recommendation, increased patient throughput at a patient care center at which the subject receives the medical intervention recommendation, and increased revenue of a patient care center at which the subject receives the medical intervention recommendation.

In some embodiments, the method can further comprise generating a dataset that provides evidence in support of an indication for a medical intervention for a condition. The medical intervention is determined by the intervention recommendation model using biomarker data for one or more subjects diagnosed with the condition as discussed above. The indication comprises values for biomarker data used by the intervention recommendation model to determine the medical intervention for one or more subjects, and is based on a medical outcome of the one or more subjects.

In some embodiments, the intervention recommendation model is stored by a primary system that is in communication with one or more third-party systems. The one or more third-party systems can be remote from the primary system. The one or more third-party systems can also be located at one or more patient care centers. In such embodiments, the intervention recommendation model can be alternatively trained and utilized between the primary system and the one or more third-party systems.

For example, in a first embodiment, the intervention recommendation model can be both trained and utilized at the primary system. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, biomarker data obtained for the subject is received from the one or more third-party systems at the primary system. Then, the medical intervention recommendation generated for the subject by the intervention recommendation model is generated at the primary system using the biomarker data for the subject.

In an alternative embodiment, the intervention recommendation model can be trained at the primary system, but utilized at the one or more third-party systems. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, the trained model is provided to the one or more third-party systems via network transmission. In some cases, the trained model is automatically provided to the third-party systems at specified time intervals or in real-time following identification of the model parameters. The biomarker data obtained for the subject is received at the model at the third-party systems, and finally, the medical intervention recommendation generated for the subject by the intervention recommendation model is generated at the third-party systems using the biomarker data for the subject.

In a third embodiment, the intervention recommendation model can conversely be trained at the one or more third-party systems, but utilized at the primary system. In such an embodiment, the intervention recommendation model is provided to the one or more third-party systems from the primary system via network transmission. Then, the intervention recommendation model receives one or more of the training samples of the training dataset at the third-party systems. At the third-party systems, the plurality of parameters of the model are identified using the received training samples. To utilize the model, the trained model is received at the primary system via network transmission. In some cases, the trained model is automatically provided to the primary system at specified time intervals or in real-time following identification of the model parameters. Then, biomarker data for the subject is received from the one or more third-party systems at the primary system, and the medical intervention recommendation for the subject is generated by the model at the primary system using the biomarker data received for the subject.

Finally, in an alternative embodiment, the intervention recommendation model can be both trained and utilized at the one or more third-party systems. In such an embodiment, the intervention recommendation model is provided to the one or more third-party systems from the primary system via network transmission. Then, the intervention recommendation model receives one or more training samples of the training dataset at the third-party systems. At the third-party systems, the plurality of parameters of the model are identified using the training samples. To utilize the model, the biomarker data obtained for the subject is received by the model at the third-party systems, and the medical intervention recommendation generated for the subject by the intervention recommendation model is generated at the third-party systems using the biomarker data for the subject.

In cases in which the intervention recommendation model is trained at the primary system, the plurality of training samples can be received at the primary system via network transmission from the one or more third-party systems. In some instances, one or more of the plurality of training samples can be received from multiple distinct third-party systems and can comprise different data formats. In such cases, the training samples can be transformed into a common data format, and the transformed training samples can be merged into a merged training dataset. This merged training dataset can then be used to identify the model parameters as discussed above.

In alternative embodiments in which the intervention recommendation model is trained at the one or more third-party systems rather than the primary system, the training samples can be received at multiple, distinct third-party systems.

Regardless of where the model is trained, the plurality of training samples can be automatically received at specified time intervals such that the parameters of the model are automatically identified at specified time intervals and such that the model is automatically updated at specified time intervals. Alternatively, the plurality of training samples can be automatically received in real-time such that the parameters of the model are automatically identified in real-time and such that the model is automatically updated in real-time.

When the model is utilized at the primary system, the biomarker data for the subject can be received at the primary system via network transmission from the one or more third-party systems. Furthermore, the medical intervention recommendation for the subject output by the intervention recommendation model can be provided by the primary system to the one or more third-party systems via network transmission.

Alternatively, when the intervention recommendation model is utilized at the one or more third-party systems, the medical intervention recommendation for the subject output by the intervention recommendation model can be provided to the subject.

In another aspect, the invention provides a non-transitory computer-readable storage medium that stores computer program instructions that, when executed by a computer processor, cause the computer processor to determine a medical intervention recommendation for the subject by performing any combination of the above method steps.

In yet another aspect, the invention provides a method for determining a medical diagnosis recommendation for a subject. The method includes obtaining biomarker data for the subject, inputting the biomarker data for the subject into a diagnostic recommendation model, and returning the medical diagnosis recommendation for the subject output by the diagnostic recommendation model. The biomarker data for the subject is input into the diagnostic recommendation model using a computer processor.

The diagnostic recommendation model comprises a plurality of parameters and a function. The function represents a relation between the biomarker data for the subject received as an input to the diagnostic recommendation model, and the medical diagnosis recommendation for the subject generated as an output of the diagnostic recommendation model based on the biomarker data for the subject and the plurality of parameters. The parameters are identified prior to use of the model, during training of the model, at least based on a training dataset. The training dataset comprises a plurality of training samples. Each training sample is associated with a retrospective subject and comprises biomarker data for the retrospective subject.

The diagnostic recommendation model is any model for which the plurality of parameters comprising the model are learned by a computer based on the training dataset. The plurality of parameters, and thus the model, are learned by a computer because it would be too difficult or too inefficient for the parameters to be identified by a human based on the training dataset due to the size and/or complexity of the training dataset. In some embodiments, the diagnostic recommendation model can be a discretely programmed model. In alternative embodiments, the diagnostic recommendation model can be learned via unsupervised learning (e.g., clustering). In further embodiments, the diagnostic recommendation model can be learned via supervised learning. For example, the diagnostic recommendation model can be a classifier or a regression model.

In embodiments in which the diagnostic recommendation model is learned via supervised learning, the diagnostic recommendation model can be trained based on outcome data. Specifically, in embodiments in which the diagnostic recommendation model is learned via supervised learning, each training sample of the training dataset can further include a medical diagnosis provided to the retrospective subject associated with the training sample and a medical outcome of the retrospective subject associated with the training sample, such that the diagnostic recommendation model is trained to optimize the medical outcome of subjects.

Biomarker data for a subject is obtained from a sample from the subject, and comprises data describing the presence or absence of one or more measurable substances in the sample. In a preferred embodiment, biomarker data can comprise at least one of genomic data, epigenomic data, transcriptomic data, proteomic data, metabolic data, and lipidomic data. In further embodiments, biomarker data can comprise a quantification of expression of each of a plurality of genes in a specified gene panel. In a preferred embodiment, a sample from a subject that is used to determine biomarker data comprises at least one of a blood sample, a urine, stool, bronchial lavage, tissue, mucus, or other bodily sample. In some embodiments, a sample from a subject that is used to determine biomarker data is collected by one or more of a FDA-cleared, commercially-available sample collection, transport, and processing device.

Biomarker data can be determined from a subject's sample using clinical laboratory equipment, an in vitro diagnostic (IVD) device, a research-use-only device, and any other means of biomarker data determination or collection. Biomarker data for a subject can be automatically received from an IVD device. In such embodiments in which the biomarker data for a subject is received from an IVD device, the biomarker data can include at least one of genomic, epigenomic, transcriptomic, proteomic, metabolomic, and lipidomic data for the subject.

In embodiments in which the biomarker data 103 comprises proteomic data, the biomarker data 103 can be determined from the subject's sample by at least one of mass spectrometry and immunoassay. In embodiments in which the biomarker data 103 comprises genomic data, the biomarker data 103 can be determined from the subject's sample by at least one of exome and whole genome nucleotide sequencing. In embodiments in which the biomarker data 103 comprises transcriptomic data, the biomarker data 103 can be determined from the subject's sample by at least one of microarray, RNA sequencing, and RT-qPCR.

A sample from a subject used to determine biomarker data can be collected at any site, and biomarker data can be determined using the collected sample at any site, prior to being input into diagnostic recommendation model. For example, a sample from a subject can be collected at a patient care center (e.g., a physician's office, a hospital), a clinical laboratory, a CLIA-certified laboratory, a research laboratory, a remote location, and any other site. Similarly, biomarker data can be determined using the collected sample at a patient care center (e.g., a physician's office, a hospital), a clinical laboratory, a CLIA-certified laboratory, a research laboratory, a remote location, and any other site. In certain embodiments, biomarker data for a subject is determined at the same site at which the sample from the subject was collected. For example, biomarker data can be obtained from a sample from a subject on-site at a patient care center at which the subject provided the sample. In alternative embodiments, biomarker data for a subject can be determined at a different site from which the sample from the subject was collected. For example, biomarker data can be obtained from a sample from a subject off-site from a patient care center at which the subject provided the sample. Biomarker data can also be obtained from any private, public, and/or commercial source. Biomarker data used to train the model can be retrospective data. On the other hand, biomarker data used to utilize the model can be prospective data.

The medical diagnosis recommendation for the subject output by the diagnostic recommendation model can include one of sepsis, septic shock, refractory septic shock, acute lung injury, acute respiratory distress syndrome, acute renal failure, acute kidney injury, trauma, burns, COVID19, pneumonia, viral infection, and post-operative conditions (e.g., conditions following open heart surgery). In some embodiments, the method further includes a step of providing a medical intervention to the subject based on the determined medical diagnosis recommendation. In such embodiments, the medical intervention can include at least one of a selection, dosage, timing, starting, stopping, and monitoring of one or more pharmaceutical compounds, drugs, and biologics. In alternative embodiments, the medical intervention can be a non-pharmaceutical intervention. In some cases, the non-pharmaceutical intervention is the collection of a biospecimen from the subject and/or the collection of electronic health record data from the subject.

The medical diagnosis recommendation output by the diagnostic recommendation model can fulfill at least one of the following conditions when compared to a standard-of-care medical diagnosis for a retrospective subject having at least one of the electronic health record data and the biomarker data of the subject: reduced morbidity of the subject, reduced mortality of the subject, increased quantity of intervention-free days of the subject, reduced time to provide the medical diagnosis recommendation to the subject, reduced time to provide the medical diagnosis recommendation to the subject, reduced cost of stay of the subject at a patient care center at which the subject receives the medical diagnosis recommendation, reduced length of stay of the subject at a patient care center at which the subject receives the medical diagnosis recommendation, reduced quantity of adverse events of the subject, improved patient quality scores of the subject, improved patient care center quality scores for a patient care center at which the subject receives the medical diagnosis recommendation, improved patient satisfaction with a patient care center at which the subject receives the medical diagnosis recommendation, increased patient throughput at a patient care center at which the subject receives the medical diagnosis recommendation, and increased revenue of a patient care center at which the subject receives the medical diagnosis recommendation.

In some embodiments, the diagnostic recommendation model is stored by a primary system that is in communication with one or more third-party systems. The one or more third-party systems can be remote from the primary system. The one or more third-party systems can also be located at one or more patient care centers. In such embodiments, the diagnostic recommendation model can be alternatively trained and utilized between the primary system and the one or more third-party systems.

For example, in a first embodiment, the diagnostic recommendation model can be both trained and utilized at the primary system. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, biomarker data obtained for the subject is received from the one or more third-party systems at the primary system. Then, the medical diagnosis recommendation generated for the subject by the diagnostic recommendation model is generated at the primary system using the biomarker data for the subject.

In an alternative embodiment, the diagnostic recommendation model can be trained at the primary system, but utilized at the one or more third-party systems. In such embodiments, the primary system receives one or more of the plurality of training samples of the training dataset from the one or more third-party systems. Then, the plurality of parameters of the model are identified at the primary system using the plurality of training samples received from the third-party systems. To utilize the model, the trained model is provided to the one or more third-party systems via network transmission. In some cases, the trained model is automatically provided to the third-party systems at specified time intervals or in real-time following identification of the model parameters. The biomarker data obtained for the subject is received at the model at the third-party systems, and finally, the medical diagnosis recommendation generated for the subject by the diagnostic recommendation model is generated at the third-party systems using the biomarker data for the subject.

In a third embodiment, the diagnostic recommendation model can conversely be trained at the one or more third-party systems, but utilized at the primary system. In such an embodiment, the diagnostic recommendation model is provided to the one or more third-party systems from the primary system via network transmission. Then, the diagnostic recommendation model receives one or more of the training samples of the training dataset at the third-party systems. At the third-party systems, the plurality of parameters of the model are identified using the received training samples. To utilize the model, the trained model is received at the primary system via network transmission. In some cases, the trained model is automatically provided to the primary system at specified time intervals or in real-time following identification of the model parameters. Then, biomarker data for the subject is received from the one or more third-party systems at the primary system, and the medical diagnosis recommendation for the subject is generated by the model at the primary system using the biomarker data received for the subject.

Finally, in an alternative embodiment, the diagnostic recommendation model can be both trained and utilized at the one or more third-party systems. In such an embodiment, the diagnostic recommendation model is provided to the one or more third-party systems from the primary system via network transmission. Then, the diagnostic recommendation model receives one or more training samples of the training dataset at the third-party systems. At the third-party systems, the plurality of parameters of the model are identified using the training samples. To utilize the model, the biomarker data obtained for the subject is received by the model at the third-party systems, and the medical diagnosis recommendation generated for the subject by the diagnostic recommendation model is generated at the third-party systems using the biomarker data for the subject.

In cases in which the diagnostic recommendation model is trained at the primary system, the plurality of training samples can be received at the primary system via network transmission from the one or more third-party systems. In some instances, one or more of the plurality of training samples can be received from multiple distinct third-party systems and can comprise different data formats. In such cases, the training samples can be transformed into a common data format, and the transformed training samples can be merged into a merged training dataset. This merged training dataset can then be used to identify the model parameters as discussed above.

In alternative embodiments in which the diagnostic recommendation model is trained at the one or more third-party systems rather than the primary system, the training samples can be received at multiple, distinct third-party systems.

Regardless of where the model is trained, the plurality of training samples can be automatically received at specified time intervals such that the parameters of the model are automatically identified at specified time intervals and such that the model is automatically updated at specified time intervals. Alternatively, the plurality of training samples can be automatically received in real-time such that the parameters of the model are automatically identified in real-time and such that the model is automatically updated in real-time.

When the model is utilized at the primary system, the biomarker data for the subject can be received at the primary system via network transmission from the one or more third-party systems. Furthermore, the medical diagnosis recommendation for the subject output by the diagnostic recommendation model can be provided by the primary system to the one or more third-party systems via network transmission.

In another aspect, the invention provides a non-transitory computer-readable storage medium that stores computer program instructions that, when executed by a computer processor, cause the computer processor to determine a medical diagnosis recommendation for the subject by performing any combination of the above method steps.

### XI. Additional Considerations

It should be noted that, as used in the specification and the appended claims, the singular forms “a,” “an,” and “the” include plural referents unless the context clearly dictates otherwise.

All references, issued patents and patent applications cited within the body of the specification are hereby incorporated by reference in their entirety, for all purposes.

The foregoing description of the embodiments of the invention has been presented for the purpose of illustration—it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Any of the steps, operations, or processes described herein can be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may include information resulting from a computing process, where the information is stored on a non-transitory, tangible computer-readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

What is claimed is:

1. A method for determining a medical intervention recommendation for a subject diagnosed with a condition, the method comprising the steps of:
obtaining electronic health record data for the subject;
obtaining biomarker data for the subject, the biomarker data obtained from a sample from the subject;
inputting, using a computer processor, the electronic health record data and the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model.

2. A method for determining a medical intervention recommendation for a subject diagnosed with a condition, the method comprising the steps of:
obtaining electronic health record data for the subject;
obtaining biomarker data for the subject, the biomarker data obtained from a sample from the subject;
inputting, using a computer processor, the electronic health record data and the biomarker data for the subject into an intervention recommendation model to generate a medical intervention recommendation for the subject,
wherein the intervention recommendation model is stored by a primary system, the primary system in communication with one or more third-party systems remote from the primary system,
and wherein the intervention recommendation model comprises:
a plurality of parameters identified by:
providing the intervention recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model.

3. A method for determining a medical intervention recommendation for a subject diagnosed with a condition, the method comprising the steps of:
obtaining electronic health record data for the subject;
automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject;
inputting, using a computer processor, the electronic health record data and the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model.

4. A method, comprising:
determining a medical intervention recommendation for a subject diagnosed with a condition by:
obtaining electronic health record data for the subject;
obtaining biomarker data for the subject, the biomarker data obtained from a sample from the subject;
inputting, using a computer processor, the electronic health record data and the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model; and
generating a dataset that provides evidence in support of an indication for a medical intervention recommendation for the condition, the medical intervention recommendation determined by the intervention recommendation model using electronic health record data and biomarker data for one or more subjects diagnosed with the condition, the indication comprising values for at least one of electronic health record data and biomarker data used by the intervention recommendation model to determine the medical intervention recommendation for one or more subjects and based on a medical outcome of the one or more subjects.

5. A method for determining a medical intervention recommendation for a subject diagnosed with a condition, the method comprising the steps of:
obtaining electronic health record data for the subject;
obtaining biomarker data for the subject, the biomarker data obtained from a sample from the subject;
inputting, using a computer processor, the electronic health record data and the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model,
wherein the medical intervention recommendation for the subject output by the intervention recommendation model fulfills at least one of the following conditions when compared to a standard-of-care medical intervention for a retrospective subject having at least one of the electronic health record data and the biomarker data of the subject:
reduced morbidity of the subject,
reduced mortality of the subject,
increased quantity of intervention-free days of the subject,
reduced time to provide the medical intervention recommendation to the subject,
reduced cost of stay of the subject at a patient care center at which the subject receives the medical intervention recommendation,
reduced length of stay of the subject at a patient care center at which the subject receives the medical intervention recommendation,
reduced quantity of adverse events of the subject,
improved patient quality scores of the subject,
improved patient care center quality scores for a patient care center at which the subject receives the medical intervention recommendation,
improved patient satisfaction with a patient care center at which the subject receives the medical intervention recommendation,
increased patient throughput at a patient care center at which the subject receives the medical intervention recommendation, and
increased revenue of a patient care center at which the subject receives the medical intervention recommendation.

6. The method of any one of claims 1-5, wherein prior to inputting the electronic health record data and the biomarker data for the subject into the intervention recommendation model, transforming the electronic heath record data and the biomarker data into a common data format.

7. The method of any one of claims 1-6, wherein each training sample of the training dataset further comprises:
a medical intervention provided to the retrospective subject associated with the training sample; and
a medical outcome of the retrospective subject following receipt of the medical intervention recommendation.

8. The method of any one of claims 1-7, wherein the intervention recommendation model is stored by a primary system, the primary system in communication with one or more third-party systems.

9. The method of claim 8, wherein the one or more third-party systems are remote from the primary system.

10. The method of any one of claims 8-9, wherein the one or more third-party systems are located at one or more patient care centers.

11. The method of any one of claims 8-10, further comprising:
receiving, from the one or more third-party systems, at the primary system, one or more of the plurality of training samples of the training dataset; and
identifying, at the primary system, the plurality of parameters using the plurality of training samples received from the one or more third-party systems,
wherein obtaining the electronic health record data and the biomarker data for the subject comprises receiving the electronic health record data and the biomarker data for the subject from the one or more third-party systems at the primary system, and
wherein the medical intervention recommendation generated for the subject by the intervention recommendation model is generated at the primary system using the electronic health record data and the biomarker data for the subject.

12. The method of any one of claims 8-10, further comprising;
receiving, from the one or more third-party systems, at the primary system, one or more of the plurality of training samples of the training dataset;
identifying, at the primary system, the plurality of parameters using the plurality of training samples received from the one or more third-party systems; and
providing the intervention recommendation model to the one or more third-party systems via network transmission,
wherein obtaining the electronic health record data and the biomarker data for the subject comprises receiving the electronic health record data and the biomarker data for the subject at the intervention recommendation model at the one or more third-party systems, and
wherein the medical intervention recommendation generated for the subject by the intervention recommendation model is generated at the one or more third-party systems using the electronic health record data and the biomarker data for the subject.

13. The method of claim 12, wherein providing the intervention recommendation model to the one or more third-party systems comprises automatically providing the intervention recommendation model to the one or more third-party systems at specified time intervals.

14. The method of any one of claims 12-13, wherein providing the intervention recommendation model to the one or more third-party systems comprises automatically providing the intervention recommendation model to the one or more third-party systems in real-time, near real-time, delayed batch or on-demand following identification of the plurality of parameters.

15. The method of any one of claims 8-10, further comprising:
providing the intervention recommendation model to the one or more third-party systems via network transmission;
receiving one or more of the plurality of training samples of the training dataset at the intervention recommendation model at the one or more third-party systems;
identifying, at the one or more third-party systems, the plurality of parameters using the training samples received at the intervention recommendation model at the one or more third-party systems;
receiving the intervention recommendation model with the identified plurality of parameters at the primary system via network transmission,
wherein obtaining the electronic health record data and the biomarker data for the subject comprises receiving the electronic health record data and the biomarker data for the subject from the one or more third-party systems at the primary system, and
wherein the medical intervention recommendation generated for the subject by the intervention recommendation model is generated at the primary system using the electronic health record data and the biomarker data for the subject.

16. The method of claim 15, wherein receiving the intervention recommendation model with the identified plurality of parameters at the primary system comprises automatically receiving the intervention recommendation model with the identified plurality of parameters at the primary system at specified time intervals.

17. The method of any one of claims 15-16, wherein receiving the intervention recommendation model with the identified plurality of parameters at the primary system comprises automatically receiving the intervention recommendation model with the identified plurality of parameters at the primary system in real-time, near real-time, delayed batch or on-demand following identification of the plurality of parameters.

18. The method of any one of claims 8-10, further comprising:
providing the intervention recommendation model to the one or more third-party systems via network transmission;
receiving one or more of the plurality of training samples of the training dataset at the intervention recommendation model at the one or more third-party systems;
identifying, at the one or more third-party systems, the plurality of parameters using the training samples received at the intervention recommendation model at the one or more third-party systems;
wherein obtaining the electronic health record data and the biomarker data for the subject comprises receiving the electronic health record data and the biomarker data for the subject at the intervention recommendation model at the one or more third-party systems, and
wherein the medical intervention recommendation generated for the subject by the intervention recommendation model is generated at the one or more third-party systems using the electronic health record data and the biomarker data for the subject.

19. The method of any one of claim 11-14, wherein the plurality of training samples are received from the one or more third-party systems at the primary system via network transmission.

20. The method of any one of claims 11-14 and 19, wherein the one or more of the plurality of training samples are received from multiple distinct third-party systems and comprise different data formats, and wherein the method further comprises:
transforming the one or more of the plurality of training samples received from the multiple distinct third-party systems into a common data format; and
merging the transformed training samples in a merged training dataset,
wherein identifying the plurality of parameters using the plurality of training samples received from the one or more third-party systems comprises identifying the plurality of parameters using the merged training dataset.

21. The method of claim 20, wherein the one or more of the plurality of training samples received from the multiple distinct third-party systems are transformed into the common data format using a publicly-available data transformation model.

22. The method of any one of claims 15-18, wherein the one or more of the plurality of training samples are received at the intervention recommendation model at multiple distinct third-party systems.

23. The method of any one of claims 11 and 15-17, wherein the electronic health record data and the biomarker data for the subject is received from the one or more third-party systems at the primary system via network transmission.

24. The method of any one of claims 11, 15-17, and 23, wherein returning the medical intervention recommendation for the subject output by the intervention recommendation model comprises providing the medical intervention recommendation for the subject to the one or more third-party systems via network transmission.

25. The method of claims 12-14 and 18, wherein returning the medical intervention recommendation for the subject output by the intervention recommendation model comprises providing the medical intervention recommendation to the subject.

26. The method of any one of claims 1-25, wherein the one or more of the plurality of training samples are automatically received at specified time intervals and the plurality of parameters are automatically identified using the received training samples at specified time intervals, such that the intervention recommendation model is automatically updated at specified time intervals.

27. The method of any one of claims 1-26, wherein the one or more of the plurality of training samples are automatically received in real-time, near real-time, delayed batch or on-demand and the plurality of parameters are automatically identified in-real time using the received training samples. such that the intervention recommendation model is automatically updated in-real time.

28. The method of any one of claims 1-27, further comprising:
generating a dataset that provides evidence in support of an indication for a medical intervention recommendation for the condition, the medical intervention recommendation determined by the intervention recommendation model using electronic health record data and biomarker data for one or more subjects diagnosed with the condition, the indication comprising values for at least one of electronic health record data and biomarker data used by the intervention recommendation model to determine the medical intervention recommendation for one or more subjects and based on a medical outcome of the one or more subjects.

29. The method of any one of claims 1-28, wherein at least one of the electronic health record data and the biomarker data are at least one of publicly-available data and commercially-available data.

30. The method of any one of claims 1-29, wherein at least one of the electronic health record data and the biomarker data for the subject or the retrospective subject are retrospective data.

31. The method of any one of claims 1-30, wherein at least one of the electronic health record data and the biomarker data for the subject are prospective data.

32. The method of any one of claims 1-31, wherein the electronic health record data is obtained from a patient care center.

33. The method of any one of claims 1-32, wherein the electronic health record data is obtained from a laboratory.

34. The method of any one of claims 1-33, wherein the biomarker data is obtained from the sample from the subject using a CLIA-certified laboratory.

35. The method of any one of claims 1-2 and 4-34, wherein the biomarker data is obtained from the sample from the subject using an in vitro diagnostic device.

36. The method of claim 35, wherein obtaining the biomarker data from the sample from the subject comprises receiving un-processed data directly from the in vitro diagnostic device.

37. The method of any one of claims 1-36, wherein the biomarker data is obtained from the sample from the subject on-site at a patient care center where the subject is located.

38. The method of any one of claims 1-36, wherein the biomarker data is obtained from the sample from the subject off-site from a patient care center where the subject is located.

39. The method of any one of claims 1-38, wherein the sample from the subject comprises a blood sample.

40. The method of any one of claims 1-38, wherein the sample from the subject comprises a urine sample.

41. The method of any one of claims 1-40, wherein the sample from the subject comprises a sample collected with one or more of a FDA-cleared, commercially-available sample collection, transport, and processing device.

42. The method of any one of claims 1-2 and 4-41, wherein obtaining biomarker data for the subject comprises obtaining, from the sample from the subject, at least one of mass spectrometry, immunoassay, exome, transcriptome, or whole genome nucleotide sequencing data for the subject.

43. The method of any one of claims 1-2 and 4-42, wherein obtaining biomarker data for the subject comprises obtaining, from the sample from the subject, proteome data for the subject.

44. The method of any one of claims 1-2 and 4-43, wherein obtaining biomarker data for the subject comprises obtaining, from the sample from the subject, metabolome data for the subject.

45. The method of any one of claims 1-2 and 4-44, wherein obtaining biomarker data for the subject comprises obtaining, from the sample from the subject, lipidome data for the subject.

46. The method of any one of claims 1-45, wherein biomarker data for the subject comprises a quantification of expression of each of a plurality of genes in a gene panel.

47. The method of any one of claims 1-46, wherein the determined medical intervention recommendation is at least one of a selection, dosage, timing, starting, stopping, and monitoring of one or more pharmaceutical compounds, drugs, and biologics.

48. The method of any one of claims 1-47, wherein the determined medical intervention recommendation is a non-pharmaceutical intervention.

49. The method of any one of claims 1-4 and 6-48, wherein the medical intervention recommendation for the subject output by the intervention recommendation model fulfills at least one of the following conditions when compared to a standard-of-care medical intervention for a retrospective subject having at least one of the electronic health record data and the biomarker data of the subject:
reduced morbidity of the subject,
reduced mortality of the subject,
increased quantity of intervention-free days of the subject,
reduced time to provide the medical intervention recommendation to the subject,
reduced cost of stay of the subject at a patient care center at which the subject receives the medical intervention recommendation,
reduced length of stay of the subject at a patient care center at which the subject receives the medical intervention recommendation,
reduced quantity of adverse events of the subject,
improved patient quality scores of the subject,
improved patient care center quality scores for a patient care center at which the subject receives the medical intervention recommendation,
increased patient throughput at a patient care center at which the subject receives the medical intervention recommendation, and
increased revenue of a patient care center at which the subject receives the medical intervention recommendation.

50. The method of any one of claims 1-49, wherein the condition comprises one of sepsis, septic shock, refractory septic shock, acute lung injury, acute respiratory distress syndrome, acute renal failure, acute kidney injury, trauma, burns, COVID19, pneumonia, viral infection, and post-operative conditions.

51. The method of any one of claims 1-50, wherein the intervention recommendation model is a machine-learned model.

52. The method of any one of claims 1-51, wherein the plurality of parameters of the intervention recommendation model are identified using the training dataset by implementing federated learning.

53. The method of any one of claims 1-52, wherein inputting, using the computer processor, the electronic health record data and the biomarker data for the subject into an intervention recommendation model comprises monitoring computational operations for satisfying a computational metric.

54. The method of claim 53, wherein responsive to monitoring that the computational metric is satisfied, scaling up or scaling down computational operations.

55. The method of claim 53 or 54, wherein the computational metric is one or more of CPU utilization exceeding or falling below a threshold value, memory utilization exceeding or falling below a specified value, number of TCP connections exceeding or falling below a specified value, number of pending computational messages exceeding or falling below a specified value.

56. A method for determining a medical diagnosis recommendation of a subject, the method comprising the steps of:
obtaining electronic health record data for the subject;
obtaining biomarker data for the subject, the biomarker data obtained from a sample from the subject;
inputting, using a computer processor, the electronic health record data and the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and
returning the medical diagnosis recommendation for the subject output by the diagnostic recommendation model.

57. A method for determining a medical diagnosis recommendation of a subject, the method comprising the steps of:
obtaining electronic health record data for the subject;
obtaining biomarker data for the subject, the biomarker data obtained from a sample from the subject;
inputting, using a computer processor, the electronic health record data and the biomarker data for the subject into a diagnostic recommendation model to generate a medical diagnosis recommendation for the subject,
wherein the diagnostic recommendation model is stored by a primary system, the primary system in communication with one or more third-party systems remote from the primary system,
and wherein the diagnostic recommendation model comprises:
a plurality of parameters identified by:
providing the diagnostic recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and
returning the medical diagnosis recommendation for the subject output by the diagnostic recommendation model.

58. A method for determining a medical diagnosis recommendation of a subject, the method comprising the steps of:
obtaining electronic health record data for the subject;
automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject;
inputting, using a computer processor, the electronic health record data and the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and
returning the medical diagnosis recommendation for the subject output by the diagnostic recommendation model.

59. A method for determining a medical diagnosis recommendation of a subject, the method comprising the steps of:
obtaining electronic health record data for the subject;
obtaining biomarker data for the subject, the biomarker data obtained from a sample from the subject;
inputting, using a computer processor, the electronic health record data and the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and
returning the medical diagnosis recommendation for the subject output by the diagnostic recommendation model,
wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject:
reduced morbidity of the subject,
reduced mortality of the subject,
increased quantity of intervention-free days of the subject,
reduced time to provide the medical diagnosis recommendation to the subject,
reduced cost of stay of the subject at a patient care center at which the subject receives the medical diagnosis recommendation,
reduced length of stay of the subject at a patient care center at which the subject receives the medical diagnosis recommendation,
reduced quantity of adverse events of the subject,
improved patient quality scores of the subject,
improved patient care center quality scores for a patient care center at which the subject receives the medical diagnosis recommendation,
increased patient throughput at a patient care center at which the subject receives the medical diagnosis recommendation, and
increased revenue of a patient care center at which the subject receives the medical diagnosis recommendation.

60. The method of any one of claims 56-59, wherein each training sample of the training dataset further comprises:
a medical diagnosis of the retrospective subject associated with the training sample; and a medical outcome of the retrospective subject following receipt of the medical diagnosis.

61. The method of any one of claims 56 and 58-60, wherein the diagnostic recommendation model is stored by a primary system, the primary system in communication with one or more third-party systems.

62. The method of claim 61, wherein the one or more third-party systems are remote from the primary system.

63. The method of any one of claims 61-62, wherein the one or more third-party systems are located at one or more patient care centers.

64. The method of any one of claims 61-63, further comprising:
receiving, from the one or more third-party systems, at the primary system, one or more of the plurality of training samples of the training dataset; and
identifying, at the primary system, the plurality of parameters using the plurality of training samples received from the one or more third-party systems,
wherein obtaining the electronic health record data and the biomarker data for the subject comprises receiving the electronic health record data and the biomarker data for the subject from the one or more third-party systems at the primary system, and
wherein the medical diagnosis recommendation generated for the subject by the diagnostic recommendation model is generated at the primary system using the electronic health record data and the biomarker data for the subject.

65. The method of any one of claims 61-64, further comprising;
receiving, from the one or more third-party systems, at the primary system, one or more of the plurality of training samples of the training dataset;
identifying, at the primary system, the plurality of parameters using the plurality of training samples received from the one or more third-party systems; and
providing the diagnostic recommendation model to the one or more third-party systems via network transmission,
wherein obtaining the electronic health record data and the biomarker data for the subject comprises receiving the electronic health record data and the biomarker data for the subject at the diagnostic recommendation model at the one or more third-party systems, and
wherein the medical diagnosis recommendation generated for the subject by the diagnostic recommendation model is generated at the one or more third-party systems using the electronic health record data and the biomarker data for the subject.

66. The method of claim 65, wherein providing the diagnostic recommendation model to the one or more third-party systems comprises automatically providing the diagnostic recommendation model to the one or more third-party systems at specified time intervals.

67. The method of any one of claims 65-66, wherein providing the diagnostic recommendation model to the one or more third-party systems comprises automatically providing the diagnostic recommendation model to the one or more third-party systems in real-time, near real-time, delayed batch or on-demand following identification of the plurality of parameters.

68. The method of any one of claims 61-63, further comprising:
providing the diagnostic recommendation model to the one or more third-party systems via network transmission;
receiving one or more of the plurality of training samples of the training dataset at the diagnostic recommendation model at the one or more third-party systems;
identifying, at the one or more third-party systems, the plurality of parameters using the training samples received at the diagnostic recommendation model at the one or more third-party systems;
receiving the diagnostic recommendation model with the identified plurality of parameters at the primary system via network transmission,
wherein obtaining the electronic health record data and the biomarker data for the subject comprises receiving the electronic health record data and the biomarker data for the subject from the one or more third-party systems at the primary system, and
wherein the medical diagnosis recommendation generated for the subject by the diagnostic recommendation model is generated at the primary system using the electronic health record data and the biomarker data for the subject.

69. The method of claim 68, wherein receiving the diagnostic recommendation model with the identified plurality of parameters at the primary system comprises automatically receiving the diagnostic recommendation model with the identified plurality of parameters at the primary system at specified time intervals.

70. The method of any one of claims 68-69, wherein receiving the diagnostic recommendation model with the identified plurality of parameters at the primary system comprises automatically receiving the diagnostic recommendation model with the identified plurality of parameters at the primary system in real-time, near real-time, delayed batch or on-demand following identification of the plurality of parameters.

71. The method of any one of claims 61-63, further comprising:
providing the diagnostic recommendation model to the one or more third-party systems via network transmission;
receiving one or more of the plurality of training samples of the training dataset at the diagnostic recommendation model at the one or more third-party systems;
identifying, at the one or more third-party systems, the plurality of parameters using the training samples received at the diagnostic recommendation model at the one or more third-party systems;
wherein obtaining the electronic health record data and the biomarker data for the subject comprises receiving the electronic health record data and the biomarker data for the subject at the diagnostic recommendation model at the one or more third-party systems, and
wherein the medical diagnosis recommendation generated for the subject by the diagnostic recommendation model is generated at the one or more third-party systems using the electronic health record data and the biomarker data for the subject.

72. The method of any one of claims 64-67, wherein the plurality of training samples are received from the one or more third-party systems at the primary system via network transmission.

73. The method of any one of claims 64-67 and 72, wherein the one or more of the plurality of training samples are received from multiple distinct third-party systems and comprise different data formats, and wherein the method further comprises:
transforming the one or more of the plurality of training samples received from the multiple distinct third-party systems into a common data format; and
merging the transformed training samples in a merged training dataset,
wherein identifying the plurality of parameters using the plurality of training samples received from the one or more third-party systems comprises identifying the plurality of parameters using the merged training dataset.

74. The method of claim 73, wherein the one or more of the plurality of training samples received from the multiple distinct third-party systems are transformed into the common data format using a publicly-available data transformation model.

75. The method of any one of claims 68-71, wherein the one or more of the plurality of training samples are received at the diagnostic recommendation model at multiple distinct third-party systems.

76. The method of any one of claims 64 and 68-70, wherein the electronic health record data and the biomarker data for the subject is received from the one or more third-party systems at the primary system via network transmission.

77. The method of any one of claims 64, 68-70, and 76, wherein returning the diagnosis for the subject output by the diagnostic recommendation model comprises providing the medical diagnosis recommendation for the subject to the one or more third-party systems via network transmission.

78. The method of any one of claims 56-77, wherein the one or more of the plurality of training samples are automatically received at specified time intervals and the plurality of parameters are automatically identified using the received training samples at specified time intervals, such that the diagnostic recommendation model is automatically updated at specified time intervals.

79. The method of any one of claims 56-78, wherein the one or more of the plurality of training samples are automatically received in real-time and the plurality of parameters are automatically identified in-real time using the received training samples. such that the diagnostic recommendation model is automatically updated in-real time.

80. The method of any one of claims 56-79, wherein at least one of the electronic health record data and the biomarker data are at least one of publicly-available data and commercially-available data.

81. The method of any one of claims 56-80, wherein at least one of the electronic health record data and the biomarker data for the subject or the retrospective subject are retrospective data.

82. The method of any one of claims 56-81, wherein at least one of the electronic health record data and the biomarker data for the subject are prospective data.

83. The method of any one of claims 56-82, wherein the electronic health record data is obtained from a patient care center.

84. The method of any one of claims 56-83, wherein the electronic health record data is obtained from a laboratory.

85. The method of any one of claims 56-84, wherein the biomarker data is obtained from the sample from the subject using a CLIA-certified laboratory.

86. The method of any one of claims 56-57 and 59-85, wherein the biomarker data is obtained from the sample from the subject using an in vitro diagnostic device.

87. The method of claim 86, wherein obtaining the biomarker data from the sample from the subject comprises receiving un-processed data directly from the in vitro diagnostic device.

88. The method of any one of claims 56-87, wherein the biomarker data is obtained from the sample from the subject on-site at a patient care center where the subject is located.

89. The method of any one of claims 56-87, wherein the biomarker data is obtained from the sample from the subject off-site from a patient care center where the subject is located.

90. The method of any one of claims 56-89, wherein the sample from the subject comprises a blood sample.

91. The method of any one of claims 56-89, wherein the sample from the subject comprises a urine sample.

92. The method of any one of claims 56-91, wherein the sample from the subject comprises a sample collected with one or more of a FDA-cleared, commercially-available sample collection, transport, and processing device.

93. The method of any one of claims 56-57 and 59-92, wherein obtaining biomarker data for the subject comprises obtaining, from the sample from the subject, at least one of mass spectrometry, immunoassay, exome, transcriptome, or whole genome nucleotide sequencing data for the subject.

94. The method of any one of claims 56-57 and 59-93, wherein obtaining biomarker data for the subject comprises obtaining, from the sample from the subject, proteome data for the subject.

95. The method of any one of claims 56-57 and 59-94, wherein obtaining biomarker data for the subject comprises obtaining, from the sample from the subject, metabolome data for the subject.

96. The method of any one of claims 56-57 and 59-95, wherein obtaining biomarker data for the subject comprises obtaining, from the sample from the subject, lipidome data for the subject.

97. The method of any one of claims 56-96, wherein biomarker data for the subject comprises a quantification of expression of each of a plurality of genes in a gene panel.

98. The method of any one of claims 56-97, further comprising providing a medical intervention recommendation to the subject based on the determined medical diagnosis recommendation, the medical intervention recommendation comprising at least one of a selection, dosage, timing, starting, stopping, and monitoring of one or more pharmaceutical compounds, drugs, and biologics.

99. The method of any one of claims 56-97, further comprising providing a medical intervention recommendation to the subject based on the determined medical diagnosis recommendation, the medical intervention recommendation comprising a non-pharmaceutical intervention.

100. The method of any one of claims 56-58 and 60-99, wherein the medical diagnosis recommendation for the subject output by the diagnostic recommendation model fulfills at least one of the following when compared to a standard-of-care medical diagnosis for a retrospective subject having at least one of the electronic health record data and the biomarker data of the subject:
reduced morbidity of the subject,
reduced mortality of the subject,
increased quantity of intervention-free days of the subject,
reduced time to provide the medical diagnosis recommendation to the subject, reduced cost of stay of the subject at a patient care center at which the subject receives the medical diagnosis recommendation,
reduced length of stay of the subject at a patient care center at which the subject receives the medical diagnosis recommendation,
reduced quantity of adverse events of the subject,
improved patient quality scores of the subject,
improved patient care center quality scores for a patient care center at which the subject receives the medical diagnosis recommendation,
increased patient throughput at a patient care center at which the subject receives the medical diagnosis recommendation, and
increased revenue of a patient care center at which the subject receives the medical diagnosis recommendation.

101. The method of any one of claims 56-100, wherein the determined medical diagnosis recommendation of the subject comprises one of sepsis, septic shock, refractory septic shock, acute lung injury, acute respiratory distress syndrome, acute renal failure, acute kidney injury, trauma, burns, COVID19, pneumonia, viral infection, and post-operative conditions.

102. The method of any one of claims 56-101, wherein the diagnostic recommendation model is a machine-learned model.

103. The method of any one of claims 56-102, wherein the plurality of parameters of the diagnostic recommendation model are identified using the training dataset by implementing federated learning.

104. The method of any one of claims 56-103, wherein inputting, using the computer processor, the electronic health record data and the biomarker data for the subject into an diagnostic recommendation model comprises monitoring computational operations for satisfying a computational metric.

105. The method of claim 104, wherein responsive to monitoring that the computational metric is satisfied, scaling up or scaling down computational operations.

106. The method of claim 104 or 105, wherein the computational metric is one or more of CPU utilization exceeding or falling below a threshold value, memory utilization exceeding or falling below a specified value, number of TCP connections exceeding or falling below a specified value, number of pending computational messages exceeding or falling below a specified value.

107. A non-transitory computer-readable storage medium storing computer program instructions that when executed by a computer processor, cause the computer processor to determine a medical intervention recommendation for a subject diagnosed with a condition by:
obtaining electronic health record data for the subject;
obtaining biomarker data for the subject, the biomarker data obtained from a sample from the subject;
inputting, using the computer processor, the electronic health record data and the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model.

108. A non-transitory computer-readable storage medium storing computer program instructions that when executed by a computer processor, cause the computer processor to determine a medical intervention recommendation for a subject diagnosed with a condition by:
obtaining electronic health record data for the subject;
obtaining biomarker data for the subject, the biomarker data obtained from a sample from the subject;
inputting, using the computer processor, the electronic health record data and the biomarker data for the subject into an intervention recommendation model to generate a medical intervention recommendation for the subject,
wherein the intervention recommendation model is stored by a primary system, the primary system in communication with one or more third-party systems remote from the primary system,
and wherein the intervention recommendation model comprises:
a plurality of parameters identified by:
providing the intervention recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model.

109. A non-transitory computer-readable storage medium storing computer program instructions that when executed by a computer processor, cause the computer processor to determine a medical intervention recommendation for a subject diagnosed with a condition by:
obtaining electronic health record data for the subject;
automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject;
inputting, using the computer processor, the electronic health record data and the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model.

110. A non-transitory computer-readable storage medium storing computer program instructions that when executed by a computer processor, cause the computer processor to:
determine a medical intervention recommendation for a subject diagnosed with a condition by:
obtaining electronic health record data for the subject;
obtaining biomarker data for the subject, the biomarker data obtained from a sample from the subject;
inputting, using the computer processor, the electronic health record data and the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model; and
generating a dataset that provides evidence in support of an indication for a medical intervention recommendation for the condition, the medical intervention recommendation determined by the intervention recommendation model using electronic health record data and biomarker data for one or more subjects diagnosed with the condition, the indication comprising values for at least one of electronic health record data and biomarker data used by the intervention recommendation model to determine the medical intervention recommendation for one or more subjects and based on a medical outcome of the one or more subjects.

111. A non-transitory computer-readable storage medium storing computer program instructions that when executed by a computer processor, cause the computer processor to determine a medical intervention recommendation for a subject diagnosed with a condition by:
obtaining electronic health record data for the subject;
obtaining biomarker data for the subject, the biomarker data obtained from a sample from the subject;
inputting, using a computer processor, the electronic health record data and the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the intervention recommendation model, and the medical intervention recommendation for the subject generated as an output of the intervention recommendation model based on the electronic health record data and the biomarker data for the subject and the plurality of parameters identified at least based on the training dataset; and
returning the medical intervention recommendation for the subject output by the intervention recommendation model,
wherein the medical intervention recommendation for the subject output by the intervention recommendation model fulfills at least one of the following conditions when compared to a standard-of-care medical intervention for a retrospective subject having at least one of the electronic health record data and the biomarker data of the subject:
reduced morbidity of the subject,
reduced mortality of the subject,
increased quantity of intervention-free days of the subject,
reduced time to provide the medical intervention recommendation to the subject,
reduced cost of stay of the subject at a patient care center at which the subject receives the medical intervention recommendation,
reduced length of stay of the subject at a patient care center at which the subject receives the medical intervention recommendation,
reduced quantity of adverse events of the subject,
improved patient quality scores of the subject,
improved patient care center quality scores for a patient care center at which the subject receives the medical intervention recommendation,
increased patient throughput at a patient care center at which the subject receives the medical intervention recommendation, and
increased revenue of a patient care center at which the subject receives the medical intervention recommendation.

112. A non-transitory computer-readable storage medium storing computer program instructions that when executed by a computer processor, cause the computer processor to determine a medical diagnosis recommendation of a subject by:
obtaining electronic health record data for the subject;
obtaining biomarker data for the subject, the biomarker data obtained from a sample from the subject;
inputting, using the computer processor, the electronic health record data and the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and
returning the medical diagnosis recommendation for the subject output by the diagnostic recommendation model.

113. A non-transitory computer-readable storage medium storing computer program instructions that when executed by a computer processor, cause the computer processor to determine a medical diagnosis recommendation of a subject by:
obtaining electronic health record data for the subject;
obtaining biomarker data for the subject, the biomarker data obtained from a sample from the subject;
inputting, using the computer processor, the electronic health record data and the biomarker data for the subject into a diagnostic recommendation model to generate a medical diagnosis recommendation for the subject,
wherein the diagnostic recommendation model is stored by the non-transitory computer-readable storage medium, the non-transitory computer-readable storage medium in communication with one or more third-party systems remote from the non-transitory computer-readable storage medium,
and wherein the diagnostic recommendation model comprises:
a plurality of parameters identified by:
providing the diagnostic recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and
returning the medical diagnosis recommendation for the subject output by the diagnostic recommendation model.

114. A non-transitory computer-readable storage medium storing computer program instructions that when executed by a computer processor, cause the computer processor to determine a medical diagnosis recommendation of a subject by:
obtaining electronic health record data for the subject;
automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject;
inputting, using the computer processor, the electronic health record data and the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and
returning the medical diagnosis recommendation for the subject output by the diagnostic recommendation model.

115. A non-transitory computer-readable storage medium storing computer program instructions that when executed by a computer processor, cause the computer processor to determine a medical diagnosis recommendation of a subject by:
obtaining electronic health record data for the subject;
obtaining biomarker data for the subject, the biomarker data obtained from a sample from the subject;
inputting, using the computer processor, the electronic health record data and the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
biomarker data for the retrospective subject, the biomarker data obtained from a sample from the retrospective subject; and
a function representing a relation between the electronic health record data and the biomarker data for the subject received as inputs to the diagnostic recommendation model, and the medical diagnosis recommendation of the subject generated as an output of the diagnostic recommendation model based on the electronic health record data and the biomarker data for the subject and the plurality of parameters identified at least based on the training dataset; and
returning the medical diagnosis recommendation for the subject output by the diagnostic recommendation model,
wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject:
reduced morbidity of the subject,
reduced mortality of the subject,
increased quantity of intervention-free days of the subject,
reduced time to provide the medical diagnosis recommendation to the subject,
reduced cost of stay of the subject at a patient care center at which the subject receives the medical diagnosis recommendation,
reduced length of stay of the subject at a patient care center at which the subject receives the medical diagnosis recommendation,
reduced quantity of adverse events of the subject,
improved patient quality scores of the subject,
improved patient care center quality scores for a patient care center at which the subject receives the medical diagnosis recommendation,
increased patient throughput at a patient care center at which the subject receives the medical diagnosis recommendation, and
increased revenue of a patient care center at which the subject receives the medical diagnosis recommendation.

116. A method for determining a medical intervention recommendation for a subject diagnosed with a condition, the method comprising the steps of:
obtaining electronic health record data for the subject;
inputting, using a computer processor, the electronic health record data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and
returning the medical intervention recommendation for the subject output by the intervention recommendation model.

117. A method for determining a medical intervention recommendation for a subject diagnosed with a condition, the method comprising the steps of:
obtaining electronic health record data for the subject;
inputting, using a computer processor, the electronic health record data for the subject into an intervention recommendation model to generate a medical intervention recommendation for the subject,
wherein the intervention recommendation model is stored by a primary system, the primary system in communication with one or more third-party systems remote from the primary system,
and wherein the intervention recommendation model comprises:
a plurality of parameters identified by:
providing the intervention recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
electronic health record data for the retrospective subject; and
a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and
returning the medical intervention recommendation for the subject output by the intervention recommendation model.

118. A method comprising:
determining a medical intervention recommendation for a subject diagnosed with a condition by:
obtaining electronic health record data for the subject;
inputting, using a computer processor, the electronic health record data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and
returning the medical intervention recommendation for the subject output by the intervention recommendation model; and
generating a dataset that provides evidence in support of an indication for a medical intervention recommendation for the condition, the medical intervention recommendation determined by the intervention recommendation model using electronic health record data for one or more subjects diagnosed with the condition, the indication comprising values for electronic health record data used by the intervention recommendation model to determine the medical intervention recommendation for one or more subjects and based on a medical outcome of the one or more subjects.

119. A method for determining a medical intervention recommendation for a subject diagnosed with a condition, the method comprising the steps of:
obtaining electronic health record data for the subject;
inputting, using a computer processor, the electronic health record data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and
returning the medical intervention recommendation for the subject output by the intervention recommendation model,
wherein the medical intervention recommendation for the subject output by the intervention recommendation model fulfills at least one of the following conditions when compared to a standard-of-care medical intervention for a retrospective subject having at least one of the electronic health record data and the biomarker data of the subject:
reduced morbidity of the subject,
reduced mortality of the subject,
increased quantity of intervention-free days of the subject,
reduced time to provide the medical intervention recommendation to the subject,
reduced cost of stay of the subject at a patient care center at which the subject receives the medical intervention recommendation,
reduced length of stay of the subject at a patient care center at which the subject receives the medical intervention recommendation,
reduced quantity of adverse events of the subject,
improved patient quality scores of the subject,
improved patient care center quality scores for a patient care center at which the subject receives the medical intervention recommendation,
increased patient throughput at a patient care center at which the subject receives the medical intervention recommendation, and
increased revenue of a patient care center at which the subject receives the medical intervention recommendation.

120. A method for determining a medical diagnosis recommendation of a subject, the method comprising the steps of:
obtaining electronic health record data for the subject;
inputting, using a computer processor, the electronic health record data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and
returning the medical diagnosis recommendation for the subject output by the diagnostic recommendation model.

121. A method for determining a medical diagnosis recommendation of a subject, the method comprising the steps of:
obtaining electronic health record data for the subject;
inputting, using a computer processor, the electronic health record data for the subject into a diagnostic recommendation model to generate a medical diagnosis recommendation for the subject,
wherein the diagnostic recommendation model is stored by a primary system, the primary system in communication with one or more third-party systems remote from the primary system,
and wherein the diagnostic recommendation model comprises:
a plurality of parameters identified by:
providing the diagnostic recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
electronic health record data for the retrospective subject; and
a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and
returning the medical diagnosis recommendation for the subject output by the diagnostic recommendation model.

122. A method for determining a medical diagnosis recommendation of a subject, the method comprising the steps of:
obtaining electronic health record data for the subject;
inputting, using a computer processor, the electronic health record data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and
returning the medical diagnosis recommendation for the subject output by the diagnostic recommendation model,
wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject:
reduced morbidity of the subject,
reduced mortality of the subject,
increased quantity of intervention-free days of the subject,
reduced time to provide the medical diagnosis recommendation to the subject,
reduced cost of stay of the subject at a patient care center at which the subject receives the medical diagnosis recommendation,
reduced length of stay of the subject at a patient care center at which the subject receives the medical diagnosis recommendation,
reduced quantity of adverse events of the subject,
improved patient quality scores of the subject,
improved patient care center quality scores for a patient care center at which the subject receives the medical diagnosis recommendation,
increased patient throughput at a patient care center at which the subject receives the medical diagnosis recommendation, and
increased revenue of a patient care center at which the subject receives the medical diagnosis recommendation.

123. A non-transitory computer-readable storage medium storing computer program instructions that when executed by a computer processor, cause the computer processor to determine a medical intervention recommendation for a subject diagnosed with a condition by:
obtaining electronic health record data for the subject;
inputting, using the computer processor, the electronic health record data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and
returning the medical intervention recommendation for the subject output by the intervention recommendation model.

124. A non-transitory computer-readable storage medium storing computer program instructions that when executed by a computer processor, cause the computer processor to determine a medical intervention recommendation for a subject diagnosed with a condition by:
obtaining electronic health record data for the subject;
inputting, using the computer processor, the electronic health record data for the subject into an intervention recommendation model to generate a medical intervention recommendation for the subject,
wherein the intervention recommendation model is stored by a primary system, the primary system in communication with one or more third-party systems remote from the primary system,
and wherein the intervention recommendation model comprises:
a plurality of parameters identified by:
providing the intervention recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
electronic health record data for the retrospective subject; and
a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and
returning the medical intervention recommendation for the subject output by the intervention recommendation model.

125. A non-transitory computer-readable storage medium storing computer program instructions that when executed by a computer processor, cause the computer processor to:
determine a medical intervention recommendation for a subject diagnosed with a condition by:
obtaining electronic health record data for the subject;
inputting, using the computer processor, the electronic health record data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and
returning the medical intervention recommendation for the subject output by the intervention recommendation model; and
generating a dataset that provides evidence in support of an indication for a medical intervention recommendation for the condition, the medical intervention recommendation determined by the intervention recommendation model using electronic health record data for one or more subjects diagnosed with the condition, the indication comprising values for electronic health record data used by the intervention recommendation model to determine the medical intervention recommendation for one or more subjects and based on a medical outcome of the one or more subjects.

126. A non-transitory computer-readable storage medium storing computer program instructions that when executed by a computer processor, cause the computer processor to determine a medical intervention recommendation for a subject diagnosed with a condition by:
obtaining electronic health record data for the subject;
inputting, using a computer processor, the electronic health record data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the intervention recommendation model, and the medical intervention recommendation for the subject generated as an output of the intervention recommendation model based on the electronic health record data for the subject and the plurality of parameters identified at least based on the training dataset; and
returning the medical intervention recommendation for the subject output by the intervention recommendation model,
wherein the medical intervention recommendation for the subject output by the intervention recommendation model fulfills at least one of the following conditions when compared to a standard-of-care medical intervention for a retrospective subject having at least one of the electronic health record data and the biomarker data of the subject:
reduced morbidity of the subject,
reduced mortality of the subject,
increased quantity of intervention-free days of the subject,
reduced time to provide the medical intervention recommendation to the subject,
reduced cost of stay of the subject at a patient care center at which the subject receives the medical intervention recommendation,
reduced length of stay of the subject at a patient care center at which the subject receives the medical intervention recommendation,
reduced quantity of adverse events of the subject,
improved patient quality scores of the subject,
improved patient care center quality scores for a patient care center at which the subject receives the medical intervention recommendation,
increased patient throughput at a patient care center at which the subject receives the medical intervention recommendation, and
increased revenue of a patient care center at which the subject receives the medical intervention recommendation.

127. A non-transitory computer-readable storage medium storing computer program instructions that when executed by a computer processor, cause the computer processor to determine a medical diagnosis recommendation of a subject by:
obtaining electronic health record data for the subject;
inputting, using the computer processor, the electronic health record data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and
returning the medical diagnosis recommendation for the subject output by the diagnostic recommendation model.

128. A non-transitory computer-readable storage medium storing computer program instructions that when executed by a computer processor, cause the computer processor to determine a medical diagnosis recommendation of a subject by:
obtaining electronic health record data for the subject;
inputting, using the computer processor, the electronic health record data for the subject into a diagnostic recommendation model to generate a medical diagnosis recommendation for the subject,
wherein the diagnostic recommendation model is stored by the non-transitory computer-readable storage medium, the non-transitory computer-readable storage medium in communication with one or more third-party systems remote from the non-transitory computer-readable storage medium,
and wherein the diagnostic recommendation model comprises:
a plurality of parameters identified by:
providing the diagnostic recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
electronic health record data for the retrospective subject; and
a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and
returning the medical diagnosis recommendation for the subject output by the diagnostic recommendation model.

129. A non-transitory computer-readable storage medium storing computer program instructions that when executed by a computer processor, cause the computer processor to determine a medical diagnosis recommendation of a subject by:
obtaining electronic health record data for the subject;
inputting, using the computer processor, the electronic health record data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
electronic health record data for the retrospective subject; and
a function representing a relation between the electronic health record data for the subject received as an input to the diagnostic recommendation model, and the medical diagnosis recommendation of the subject generated as an output of the diagnostic recommendation model based on the electronic health record data for the subject and the plurality of parameters identified at least based on the training dataset; and
returning the medical diagnosis recommendation for the subject output by the diagnostic recommendation model,
wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject:
reduced morbidity of the subject,
reduced mortality of the subject,
increased quantity of intervention-free days of the subject,
reduced time to provide the medical diagnosis recommendation to the subject,
reduced cost of stay of the subject at a patient care center at which the subject receives the medical diagnosis recommendation,
reduced length of stay of the subject at a patient care center at which the subject receives the medical diagnosis recommendation,
reduced quantity of adverse events of the subject,
improved patient quality scores of the subject,
improved patient care center quality scores for a patient care center at which the subject receives the medical diagnosis recommendation,
increased patient throughput at a patient care center at which the subject receives the medical diagnosis recommendation, and
increased revenue of a patient care center at which the subject receives the medical diagnosis recommendation.

130. A method for determining a medical intervention recommendation for a subject diagnosed with a condition, the method comprising the steps of:
obtaining biomarker data for the subject, the biomarker data obtained from a sample from the subject;
inputting, using a computer processor, the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model.

131. A method for determining a medical intervention recommendation for a subject diagnosed with a condition, the method comprising the steps of:
obtaining biomarker data for the subject, the biomarker data obtained from a sample from the subject;
inputting, using a computer processor, the biomarker data for the subject into an intervention recommendation model to generate a medical intervention recommendation for the subject,
wherein the intervention recommendation model is stored by a primary system, the primary system in communication with one or more third-party systems remote from the primary system,
and wherein the intervention recommendation model comprises:
a plurality of parameters identified by:
providing the intervention recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model.

132. A method for determining a medical intervention recommendation for a subject diagnosed with a condition, the method comprising the steps of:
automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject;
inputting, using a computer processor, the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model.

133. A method comprising:
determining a medical intervention recommendation for a subject diagnosed with a condition by:
obtaining biomarker data for the subject, the biomarker data obtained from a sample from the subject;
inputting, using a computer processor, the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model; and
generating a dataset that provides evidence in support of an indication for a medical intervention recommendation for the condition, the medical intervention recommendation determined by the intervention recommendation model using biomarker data for one or more subjects diagnosed with the condition, the indication comprising values for biomarker data used by the intervention recommendation model to determine the medical intervention recommendation for one or more subjects and based on a medical outcome of the one or more subjects.

134. A method for determining a medical intervention recommendation for a subject diagnosed with a condition, the method comprising the steps of:
obtaining biomarker data for the subject, the biomarker data obtained from a sample from the subject;
inputting, using a computer processor, the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model,
wherein the medical intervention recommendation for the subject output by the intervention recommendation model fulfills at least one of the following conditions when compared to a standard-of-care medical intervention for a retrospective subject having at least one of the electronic health record data and the biomarker data of the subject:
reduced morbidity of the subject,
reduced mortality of the subject,
increased quantity of intervention-free days of the subject,
reduced time to provide the medical intervention recommendation to the subject,
reduced cost of stay of the subject at a patient care center at which the subject receives the medical intervention recommendation,
reduced length of stay of the subject at a patient care center at which the subject receives the medical intervention recommendation,
reduced quantity of adverse events of the subject,
improved patient quality scores of the subject,
improved patient care center quality scores for a patient care center at which the subject receives the medical intervention recommendation,
increased patient throughput at a patient care center at which the subject receives the medical intervention recommendation, and
increased revenue of a patient care center at which the subject receives the medical intervention recommendation.

135. A method for determining a medical diagnosis recommendation of a subject, the method comprising the steps of:
obtaining biomarker data for the subject, the biomarker data obtained from a sample from the subject;
inputting, using a computer processor, the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and
returning the medical diagnosis recommendation for the subject output by the diagnostic recommendation model.

136. A method for determining a medical diagnosis recommendation of a subject, the method comprising the steps of:
obtaining biomarker data for the subject, the biomarker data obtained from a sample from the subject;
inputting, using a computer processor, the biomarker data for the subject into a diagnostic recommendation model to generate a medical diagnosis recommendation for the subject,
wherein the diagnostic recommendation model is stored by a primary system, the primary system in communication with one or more third-party systems remote from the primary system,
and wherein the diagnostic recommendation model comprises:
a plurality of parameters identified by:
providing the diagnostic recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and
returning the medical diagnosis recommendation for the subject output by the diagnostic recommendation model.

137. A method for determining a medical diagnosis recommendation of a subject, the method comprising the steps of:
automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject;
inputting, using a computer processor, the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and
returning the medical diagnosis recommendation for the subject output by the diagnostic recommendation model.

138. A method for determining a medical diagnosis recommendation of a subject, the method comprising the steps of:
obtaining biomarker data for the subject, the biomarker data obtained from a sample from the subject;
inputting, using a computer processor, the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and
returning the medical diagnosis recommendation for the subject output by the diagnostic recommendation model,
wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject:
reduced morbidity of the subject,
reduced mortality of the subject,
increased quantity of intervention-free days of the subject,
reduced time to provide the medical diagnosis recommendation to the subject,
reduced cost of stay of the subject at a patient care center at which the subject receives the medical diagnosis recommendation,
reduced length of stay of the subject at a patient care center at which the subject receives the medical diagnosis recommendation,
reduced quantity of adverse events of the subject,
improved patient quality scores of the subject,
improved patient care center quality scores for a patient care center at which the subject receives the medical diagnosis recommendation,
increased patient throughput at a patient care center at which the subject receives the medical diagnosis recommendation, and
increased revenue of a patient care center at which the subject receives the medical diagnosis recommendation.

139. A non-transitory computer-readable storage medium storing computer program instructions that when executed by a computer processor, cause the computer processor to determine a medical intervention recommendation for a subject diagnosed with a condition by:
obtaining biomarker data for the subject, the biomarker data obtained from a sample from the subject;
inputting, using the computer processor, the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model.

140. A non-transitory computer-readable storage medium storing computer program instructions that when executed by a computer processor, cause the computer processor to determine a medical intervention recommendation for a subject diagnosed with a condition by:
obtaining biomarker data for the subject, the biomarker data obtained from a sample from the subject;
inputting, using the computer processor, the biomarker data for the subject into an intervention recommendation model to generate a medical intervention recommendation for the subject,
wherein the intervention recommendation model is stored by a primary system, the primary system in communication with one or more third-party systems remote from the primary system,
and wherein the intervention recommendation model comprises:
a plurality of parameters identified by:
providing the intervention recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model.

141. A non-transitory computer-readable storage medium storing computer program instructions that when executed by a computer processor, cause the computer processor to determine a medical intervention recommendation for a subject diagnosed with a condition by:
automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject;
inputting, using the computer processor, the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model.

142. A non-transitory computer-readable storage medium storing computer program instructions that when executed by a computer processor, cause the computer processor to:
determine a medical intervention recommendation for a subject diagnosed with a condition by:
obtaining biomarker data for the subject, the biomarker data obtained from a sample from the subject;
inputting, using the computer processor, the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model; and
generating a dataset that provides evidence in support of an indication for a medical intervention recommendation for the condition, the medical intervention recommendation determined by the intervention recommendation model using biomarker data for one or more subjects diagnosed with the condition, the indication comprising values for biomarker data used by the intervention recommendation model to determine the medical intervention recommendation for one or more subjects and based on a medical outcome of the one or more subjects.

143. A non-transitory computer-readable storage medium storing computer program instructions that when executed by a computer processor, cause the computer processor to determine a medical intervention recommendation for a subject diagnosed with a condition by:
obtaining biomarker data for the subject, the biomarker data obtained from a sample from the subject;
inputting, using a computer processor, the biomarker data for the subject into an intervention recommendation model to generate a medical intervention recommendation for the subject, the intervention recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the intervention recommendation model, and the medical intervention recommendation for the subject generated as an output of the intervention recommendation model based on the biomarker data for the subject and the plurality of parameters identified at least based on the training dataset; and
returning the medical intervention recommendation for the subject output by the intervention recommendation model,
wherein the medical intervention recommendation for the subject output by the intervention recommendation model fulfills at least one of the following conditions when compared to a standard-of-care medical intervention for a retrospective subject having at least one of the electronic health record data and the biomarker data of the subject:
reduced morbidity of the subject,
reduced mortality of the subject,
increased quantity of intervention-free days of the subject,
reduced time to provide the medical intervention recommendation to the subject,
reduced cost of stay of the subject at a patient care center at which the subject receives the medical intervention recommendation,
reduced length of stay of the subject at a patient care center at which the subject receives the medical intervention recommendation,
reduced quantity of adverse events of the subject,
improved patient quality scores of the subject,
improved patient care center quality scores for a patient care center at which the subject receives the medical intervention recommendation,
increased patient throughput at a patient care center at which the subject receives the medical intervention recommendation, and
increased revenue of a patient care center at which the subject receives the medical intervention recommendation.

144. A non-transitory computer-readable storage medium storing computer program instructions that when executed by a computer processor, cause the computer processor to determine a medical diagnosis recommendation of a subject by:
obtaining biomarker data for the subject, the biomarker data obtained from a sample from the subject;
inputting, using the computer processor, the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and
returning the medical diagnosis recommendation for the subject output by the diagnostic recommendation model.

145. A non-transitory computer-readable storage medium storing computer program instructions that when executed by a computer processor, cause the computer processor to determine a medical diagnosis recommendation of a subject by:
obtaining biomarker data for the subject, the biomarker data obtained from a sample from the subject;
inputting, using the computer processor, the biomarker data for the subject into a diagnostic recommendation model to generate a medical diagnosis recommendation for the subject,
wherein the diagnostic recommendation model is stored by the non-transitory computer-readable storage medium, the non-transitory computer-readable storage medium in communication with one or more third-party systems remote from the non-transitory computer-readable storage medium,
and wherein the diagnostic recommendation model comprises:
a plurality of parameters identified by:
providing the diagnostic recommendation model to the one or more third-party systems via network transmission;
identifying, at the one or more third-party systems, the plurality of parameters using a training dataset received at the one or more third-party systems, the training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and
returning the medical diagnosis recommendation for the subject output by the diagnostic recommendation model.

146. A non-transitory computer-readable storage medium storing computer program instructions that when executed by a computer processor, cause the computer processor to determine a medical diagnosis recommendation of a subject by:
automatically receiving biomarker data for the subject from an in vitro diagnostic device that identified the biomarker data for the subject from a sample from the subject, the biomarker data comprising at least one of genomic, epigenomic, transcriptomic, proteomic, metabolomic, and lipidomic data for the subject;
inputting, using the computer processor, the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and
returning the medical diagnosis recommendation for the subject output by the diagnostic recommendation model.

147. A non-transitory computer-readable storage medium storing computer program instructions that when executed by a computer processor, cause the computer processor to determine a medical diagnosis recommendation of a subject by:
obtaining biomarker data for the subject, the biomarker data obtained from a sample from the subject;
inputting, using the computer processor, the biomarker data for the subject into a diagnostic recommendation model to generate a medical diagnosis recommendation for the subject, the diagnostic recommendation model comprising:
a plurality of parameters identified at least based on a training dataset comprising a plurality of training samples, each training sample associated with a retrospective subject and comprising:
biomarker data for the retrospective subject, the biomarker data obtained from a sample from the retrospective subject; and
a function representing a relation between the biomarker data for the subject received as an input to the diagnostic recommendation model, and the medical diagnosis recommendation of the subject generated as an output of the diagnostic recommendation model based on the biomarker data for the subject and the plurality of parameters identified at least based on the training dataset; and
returning the medical diagnosis recommendation for the subject output by the diagnostic recommendation model,
wherein the medical diagnosis recommendation for the subject output by the diagnostic recommendation model fulfills at least one of the following conditions when compared to a standard-of-care medical diagnosis for a retrospective subject having at least one of the electronic health record data and the biomarker data of the subject:
reduced morbidity of the subject,
reduced mortality of the subject,
increased quantity of intervention-free days of the subject,
reduced time to provide the medical diagnosis recommendation to the subject,
reduced cost of stay of the subject at a patient care center at which the subject receives the medical diagnosis recommendation,
reduced length of stay of the subject at a patient care center at which the subject receives the medical diagnosis recommendation,
reduced quantity of adverse events of the subject,
improved patient quality scores of the subject,
improved patient care center quality scores for a patient care center at which the subject receives the medical diagnosis recommendation,
increased patient throughput at a patient care center at which the subject receives the medical diagnosis recommendation, and
increased revenue of a patient care center at which the subject receives the medical diagnosis recommendation.
